(19) United States
(12) Patent Application Publication
(10) Pub. No.: US 2022/0235315 A1
(43) Pub. Date: Jul. 28, 2022

(54) TARGETING SEQUENCES FOR PAENIBACILLUS-BASED ENDOSPORE DISPLAY PLATFORM

(71) Applicant: Bayer CropScience LP, St. Louis, MO (US)

(72) Inventors: Damian CURTIS, Davis, CA (US); Kyle TIPTON, San Jose, CA (US)

(21) Appl. No.: 17/610,250

(22) PCT Filed: May 15, 2020
(86) PCT No.: PCT/US2020/033018

§ 371 Date: Nov. 10, 2021

Related U.S. Application Data
(60) Provisional application No. 62/848,533, filed on May 15, 2019.

(51) Int. Cl. C12N 1/20; C07K 14/195; C12N 11/16; C12N 3/00; C12N 15/63

(52) U.S. Cl. CPC C12N 1/205; C07K 14/195; C07K 2319/035; C12N 3/00; C12N 15/63; C12N 11/16

(57) ABSTRACT

Signal sequences useful for targeting proteins and peptides to the surface of endospores produced by Paenibacillus family members and methods of using the same are provided. The display of heterologous molecules, such as peptides, polypeptides and other recombinant constructs, on the spore surface of Paenibacillus family members, using particular N-terminal targeting sequences and derivatives of the same, and likewise are provided.

### CROSS-REFERENCE TO RELATED APPLICATIONS

This application claims priority to U.S. Provisional Patent Application No. 62/848,533, filed on May 15, 2019, the entire contents of which are incorporated herein by reference in its entirety.

### REFERENCE TO SEQUENCE LISTING SUBMITTED ELECTRONICALLY

The official copy of the sequence listing is submitted electronically via EFS-Web as an ASCII-formatted sequence listing with a file named BCS199006_WO_ST25.txt created on May 14, 2020, and having a size of 85 kilobytes, and is filed concurrently with the specification. The sequence listing contained in this ASCII-formatted document is part of the specification and is herein incorporated by reference in its entirety.

### TECHNICAL FIELD

The disclosure is generally directed to endospore display platforms, related display methods, spore surface targeting sequences and fusion protein constructs comprising the same, recombinant endospore compositions, and methods for identifying spore surface-targeting sequences in Paenibacillus and other bacterial genera that are useful for various applications such as the delivery of a heterologous molecule of interest to a plant, seed or field.

### BACKGROUND OF THE DISCLOSURE

Modern agricultural techniques rely heavily on compositions that promote or enhance plant health and growth in order to improve the yield and quality of crops. Such compositions generally include organic or inorganic fertilizers, nutrients and other chemical compounds that promote proper plant growth and development. However, it is well established that long-term or overuse of many of these compositions may result in negative side effects, such as soil acidification or destabilization of the nutrient balance in the soil. Moreover, overuse may result in the enrichment of harmful end-products in crops grown for human consumption.

Modern farms also typically rely on the use of a wide variety of chemicals (e.g., insecticides, herbicides, bactericides, nematicides, and fungicides) to control pests and ensure a high yield of commercially-grown crops. Many of these chemical compounds exhibit broad activity and may be potentially harmful to humans and animals in high concentrations. In addition, some chemical compounds exhibit off-target effects. Moreover, at least some of these synthetic compounds are non-biodegradable. In recent years, there has been increasing pressure from consumers for agricultural products that have been raised and harvested with reduced or no exposure to synthetic insecticides or fungicides. A further problem arising with the use of synthetic insecticides or fungicides is that the repeated and/or exclusive use often leads to selection of resistant pests. Normally, resistant pests are also cross-resistant against other active ingredients having the same mode of action. As a result, pest control compositions and compounds are difficult and expensive to develop (e.g., due to safety concerns and the rapid development of resistance).

Genetic engineering methods are used to promote plant growth and/or health without reliance on synthetic chemicals. For example, crops can be modified to introduce or modify genes related to plant growth and/or health, and/or to introduce genes that encode natural or synthetic pest control agents. Transgenes may be introduced into a target plant using a viral vector. In recent years, there has been some success reported using bacteria for delivery of recombinant proteins to plants. However, to date, such success is largely limited to members of the Bacillaceae family and more specifically, Bacillus subtilis, which is the most well-characterized, Gram-positive bacteria and the primary bacterial model for sporulation research. The focus on B. subtilis as a delivery and expression platform is further due to the fact that the B. subtilis genome and biological pathways related to protein synthesis and secretion are well understood. However, due to the high degree of genetic diversity among bacteria, research findings based on B. subtilis studies are often not directly translatable to members inside and outside the Bacillaceae family.

Accordingly, while certain methods of delivering heterologous genetic materials are known, there is a need in the art for developing new delivery and expression platforms for such genetic materials.

### BRIEF SUMMARY OF EMBODIMENTS OF THE DISCLOSURE

The disclosure describes methods, compositions and genetic constructs that address the needs identified above by, for example, providing, among other things, a new platform for delivering recombinant enzymes and other molecules of interest (e.g., peptide, protein) to an environment (e.g., plant, field) using spore-forming members of the Paenibacillus genus. The disclosure also provides methods of identifying spore surface-targeting sequences in Paenibacillus and other bacterial genera.

In one aspect, the disclosure provides recombinant endospore-producing Paenibacillus cells that express a fusion protein comprising: (i) at least one heterologous protein or peptide that confers or modifies a plant trait or attribute (e.g., an enzyme involved in the production or activation of a plant growth stimulating compound; an enzyme that degrades or modifies a bacterial, fungal, or plant nutrient source; a microbicidal or microbiostatic compound; or an enzyme, protein, or peptide that protects a plant from a pathogen or a pest); and (ii) an N-terminal targeting sequence that localizes the fusion protein to the spore surface of the Paenibacillus spores. This general composition may further include additional components (e.g., that promote plant growth and/or health). Moreover, particular embodiments of the methods discloses herein provide for an efficient high-throughput screening of heterologous proteins and peptide that confer or otherwise modify plant traits or attributes.

In an alternative aspect, the disclosure provides a nucleic acid molecule encoding a fusion protein, comprising (a) a first polynucleotide sequence encoding an N-terminal signal peptide, operably linked to (b) a second polynucleotide sequence encoding a polypeptide heterologous to the N-terminal signal peptide, wherein the first polynucleotide sequence comprises: (i) a polynucleotide sequence encoding an N-terminal signal peptide; (ii) a polynucleotide sequence having at least 50%, 60%, 70%, 80% or 90% sequence identity with SEQ ID NO: 1, 3, 5, 7 or 9; or (iii) a polynucleotide sequence comprising a fragment of at least 5, 10, 15, 20, 25, 30, 35, 40, 45, 50, 55 or 60 consecutive nucleotides of SEQ ID NO: 1, 3, 5, 7 or 9; wherein the N-terminal signal peptide is capable of targeting the fusion protein to the spore surface of a Paenibacillus endospore.

In an alternative aspect, the disclosure provides a nucleic acid molecule encoding a fusion protein, comprising (a) a first polynucleotide sequence encoding an N-terminal signal peptide, operably linked to (b) a second polynucleotide sequence encoding a polypeptide heterologous to the N-terminal signal peptide, wherein the first polynucleotide sequence comprises: (i) SEQ ID NO: 33, 39, 41, or 45; (ii) a polynucleotide sequence encoding an N-terminal signal peptide having at least 50%, 55%, 60%, 65%, 70%, 75%, 80%, 85%, 90%, 95%, 96%, 97%, 98% or 99% sequence identity with SEQ ID NO: 33, 39, 41, or 45; (iii) a polynucleotide sequence encoding a polypeptide sequence having SEQ ID NO: 34, 40, 42, 46, or 49; or (iv) a polynucleotide sequence encoding one or more of the following polypeptide sequences: “MVVLSTGPIANDPVLGVRPTQLVTVKIDNRDSVNSSI VLIEGFILNGSRTLYVQQLVVVGPNAVITRNFFANVDAFEFVFTTSGPAENETQISVWGK DAL” (SEQ ID NO: 34), “MFTTSGPAENETQISVWGKDAL” (SEQ ID NO: 40), “MTQISVWGKDAL” (SEQ ID NO: 42), or “MQISVWGK(D/N)” (SEQ ID NO: 49); wherein in each case (i)-(iv) the N-terminal signal peptide is capable of targeting the fusion protein to the spore surface of a Paenibacillus endospore.

In an alternative aspect, the first polynucleotide sequence comprises a plurality of N-terminal targeting sequences, wherein each N-terminal targeting sequence is independently selected from “MVVLSTGPIANDPVLGVRPTQLVTVKIDNRDSVNS SIVLIEGFILNGSRTL YVQQLVVVGPNAVITRNFFANVDAFEFVFTTSGPAENETQISVWGKDAL” (SEQ ID NO: 34), “MFTTSGPAENETQISVWGKDAL” (SEQ ID NO: 40), “MTQISVWGKDAL” (SEQ ID NO: 42), or “MQISVWGK(D/N)” (SEQ ID NO: 49), provided that at least one such N-terminal targeting sequence is capable of targeting the fusion protein to the spore surface of a Paenibacillus endospore.” In some alternative aspects, two or more of the N-terminal targeting sequences are adjacent to each other, in others, two or more of the N-terminal targeting sequences are separated by an intervening spacer sequence comprising at least 5, 10, 15, 20, 25, 30, 35 or 40 amino acids.

In an alternative aspect, the first polynucleotide sequence consists essentially of a polynucleotide encoding any one of: “MVVLSTGPIANDPVLGVRPTQLVTVKIDNRD SVNSSIVLIEGFILNGSRTLYVQQLVVVGPNAVITRNFFANVDAFEFVFTTSGPAENETQI SVWGKDAL” (SEQ ID NO: 34), “MFTTSGPAENETQISVWGKDAL” (SEQ ID NO: 40), “MTQISVW GKDAL” (SEQ ID NO: 42), or “MQISVWGK(D/N)” (SEQ ID NO: 49).

In an alternative aspect, the first polynucleotide sequence encodes a polypeptide sequence of at most 10, 20, 30, or 40 amino acids in length, which comprises the polypeptide sequence of any one of: “MFTTSGPAENETQISVWGKDAL” (SEQ ID NO: 40), “MTQISVW GKDAL” (SEQ ID NO: 42), or “MQISVWGK(D/N)” (SEQ ID NO: 49).

In an alternative aspect, the first polynucleotide sequence encodes a polypeptide sequence of at most 10, 20, 30, or 40 amino acids in length, and comprises a polynucleotide encoding a truncated form of “MFTTSGPAENETQISVWGKDAL” (SEQ ID NO: 40), “MTQISVWGKDAL” (SEQ ID NO: 42), or “MQISVWGK(D/N)” (SEQ ID NO: 49), wherein the truncated form omits the N-terminal methionine residue.

In an alternative aspect, the first polynucleotide sequence comprises or consists of a sequence encoding the polypeptide sequence of any one of: “MFTTSGPAENETQIS VWGKDAL” (SEQ ID NO: 40), “MTQISVWGKDAL” (SEQ ID NO: 42), or “MQISVWG K(D/N)” (SEQ ID NO: 49).

In an alternative aspect, the first polynucleotide sequence comprises or consists of a codon-optimized polynucleotide sequence having at least 50%, 60%, 70%, 80% or 90% sequence identity with SEQ ID NO: 33, 39, 41, or 45, or a fragment thereof, which is expressed at a higher rate or level in the Paenibacillus endospore compared to the corresponding unoptimized sequence under identical conditions.

In an alternative aspect, the first polynucleotide comprises or consists of a sequence encoding a contiguous sequence of at least 5, 6, 7, 8, 9, 10, 11, 12, 13, 14, 15, 16, 17, 18, 19, 20, 21, 22, 23, 24, or 25 amino acids that is identical to a contiguous sequence of the same number of amino acids of any of the individual polypeptide sequences of SEQ ID NO: 34, 40, 42, 46, or 49.

In another alternative aspect, the first polynucleotide sequence consists essentially of a sequence encoding amino acids 80-90, 90-100, or 91-98 of SEQ ID NO: 2.

In another alternative aspect, the first polynucleotide sequence encodes a polypeptide sequence that comprises or consists of at most 20, 30, or 40 amino acids, that include any of the following polypeptide sequences: “FTTSGPAENETQISV WGKDAL” (amino acids 80-100 of SEQ ID NO: 2) (as to the polypeptide sequences having 30 or 40 amino acids), “TQISVWGKDAL” (amino acids 90-100 of SEQ ID NO: 2), or “QISVWGK(D/N)”. In a further aspect, such encoded polypeptide sequence with at most 20, 30 or 40 amino acids has less than 50%, 55%, 60%, 65% or 70% sequence identity to SEQ ID NO: 2 as to the portions of such first polypeptide sequence that do not correspond to amino acids 80-100 of SEQ ID NO: 2, amino acids 90-100 of SEQ ID NO: 2 or QISVWGK(D/N), as applicable.

In another alternative aspect, the disclosure provides a nucleic acid molecule encoding a fusion protein, comprising (a) a first polynucleotide sequence encoding an N-terminal signal peptide, operably linked to (b) a second polynucleotide sequence encoding a polypeptide heterologous to the N-terminal signal peptide, wherein the first polynucleotide sequence comprises: (i) a polynucleotide sequence having at least 50%, 60%, 70%, 80% or 90% sequence identity with SEQ ID NO: 1, 3, 5, 7 or 9 or (ii) a polynucleotide sequence comprising a fragment of at least 5, 10, 15, 20, 25, 30, 35, 40, 45, 50, 55 or 60 consecutive nucleotides of SEQ ID NO: 1, 3, 5, 7 or 9; wherein the N-terminal signal peptide is capable of targeting the fusion protein to the spore surface of a Paenibacillus endospore.

In some aspects, the polypeptide heterologous to the N-terminal signal peptide comprises: (a) at least one of a plant growth or immune stimulating protein; (b) an enzyme; (c) a protein; (d) a polypeptide heterologous to Paenibacillus; or (e) a therapeutic protein. In selected aspects, the nucleic acid molecule further comprising a third polynucleotide sequence, encoding: (a) a polypeptide comprising one or more protease cleavage sites, wherein the polypeptide is positioned between the N-terminal signal peptide and the polypeptide heterologous to the N-terminal signal peptide; (b) a polypeptide comprising a selectable marker; (c) a polypeptide comprising a visualization marker; (d) a polypeptide comprising a protein recognition/purification domain; or (e) a polypeptide comprising a flexible linker element, which connects the N-terminal signal peptide and the polypeptide heterologous to the N-terminal signal peptide.

In some aspects, the Paenibacillus endospore is an endospore formed by a Paenibacillus species, comprising: Paenibacillus sp. NRRL B-50972, Paenibacillus terrae, Paenibacillus polymyxa, or Paenibacillus peoriae.

In other aspects, the Paenibacillus endospore is an endospore formed by a Paenibacillus species, comprising: Paenibacillus abyssi, Paenibacillus aceti, Paenibacillus aestuarii, Paenibacillus agarexedens, Paenibacillus agaridevorans, Paenibacillus alginolyticus, Paenibacillus algorifonticola, Paenibacillus alkaliterrae, Paenibacillus alvei, Paenibacillus amylolyticus, Paenibacillus anaericanus, Paenibacillus antarcticus, Paenibacillus apiarius, Paenibacillus arachidis, Paenibacillus assamensis, Paenibacillus azoreducens, Paenibacillus azotofixans, Paenibacillus baekrokdamisoli, Paenibacillus barcinonensis, Paenibacillus barengoltzii, Paenibacillus borealis, Paenibacillus bovis, Paenibacillus brasilensis, Paenibacillus camelliae, Paenibacillus campinasensis, Paenibacillus castaneae, Paenibacillus catalpae, Paenibacillus cathormii, Paenibacillus cavernae, Paenibacillus cellulosilyticus, Paenibacillus cellulositrophicus, Paenibacillus chartarius, Paenibacillus chibensis, Paenibacillus chinjuensis, Paenibacillus chitinolyticus, Paenibacillus chondroitinus, Paenibacillus chungangensis, Paenibacillus cineris, Paenibacillus cisolokensis, Paenibacillus contaminans, Paenibacillus cookii, Paenibacillus cucumis, Paenibacillus curdlanolyticus, Paenibacillus daejeonensis, Paenibacillus darwinianus, Paenibacillus dauci, Paenibacillus dendritiformis, Paenibacillus dongdonensis, Paenibacillus doosanensis, Paenibacillus durus, Paenibacillus edaphicus, Paenibacillus ehimensis, Paenibacillus elgii, Paenibacillus endophyticus, Paenibacillus etheri, Paenibacillus faecis, Paenibacillus favisporus, Paenibacillus ferrarius, Paenibacillus filicis, Paenibacillus fonticola, Paenibacillus forsythias, Paenibacillus frigoriresistens, Paenibacillus gansuensis, Paenibacillus gelatinilyticus, Paenibacillus ginsengarvi, Paenibacillus ginsengihumi, Paenibacillus ginsengisoli, Paenibacillus glacialis, Paenibacillus glucanolyticus, Paenibacillus glycanilyticus, Paenibacillus gordonae, Paenibacillus graminis, Paenibacillus granivorans, Paenibacillus guangzhouensis, or Paenibacillus harenae.

In some aspects, the Paenibacillus endospore is an endospore formed by a Paenibacillus species, comprising: Paenibacillus hemerocallicola, Paenibacillus hispanicus, Paenibacillus hodogayensis, Paenibacillus hordei, Paenibacillus humicus, Paenibacillus hunanensis, Paenibacillus illinoisensis, Paenibacillus jamilae, Paenibacillus jilunlii, Paenibacillus kobensis, Paenibacillus koleovorans, Paenibacillus konsidensis, Paenibacillus koreensis, Paenibacillus kribbensis, Paenibacillus kyungheensis, Paenibacillus lactis, Paenibacillus larvae, Paenibacillus larvae, Paenibacillus larvae, Paenibacillus lautus, Paenibacillus lemnae, Paenibacillus lentimorbus, Paenibacillus lentus, Paenibacillus liaoningensis, Paenibacillus lupini, Paenibacillus macerans, Paenibacillus macquariensis, Paenibacillus macquariensis, Paenibacillus macquariensis, Paenibacillus marchantiophytorum, Paenibacillus marinisediminis, Paenibacillus massiliensis, Paenibacillus medicaginis, Paenibacillus mendelii, Paenibacillus methanolicus, Paenibacillus montaniterrae, Paenibacillus motobuensis, Paenibacillus mucilaginosus, Paenibacillus nanensis, Paenibacillus nap hthalenovorans, Paenibacillus nasutitermitis, Paenibacillus nematophilus, Paenibacillus nicotianae, Paenibacillus oceanisediminis, Paenibacillus odorifer, Paenibacillus oenotherae, Paenibacillus oryzae, Paenibacillus pabuli, Paenibacillus panacisoli, Paenibacillus panaciterrae, Paenibacillus pasadenensis, Paenibacillus pectinilyticus, Paenibacillus periandrae, or Paenibacillus phoenicis.

In some aspects, the Paenibacillus endospore is an endospore formed by a Paenibacillus species, comprising: Paenibacillus phyllosphaerae, Paenibacillus physcomitrellae, Paenibacillus pini, Paenibacillus pinihumi, Paenibacillus pinesoli, Paenibacillus pocheonensis, Paenibacillus popilliae, Paenibacillus populi, Paenibacillus prosopidis, Paenibacillus provencensis, Paenibacillus pueri, Paenibacillus puldeungensis, Paenibacillus pulvifaciens, Paenibacillus purispatii, Paenibacillus qingshengii, Paenibacillus quercus, Paenibacillus radicis, Paenibacillus relictisesami, Paenibacillus residui, Paenibacillus rhizoryzae, Paenibacillus rhizosphaerae, Paenibacillus rigui, Paenibacillus riograndensis, Paenibacillus ripae, Paenibacillus sabinae, Paenibacillus sacheonensis, Paenibacillus salinicaeni, Paenibacillus sanguinis, Paenibacillus sediminis, Paenibacillus segetis, Paenibacillus selenii, Paenibacillus selenitireducens, Paenibacillus senegalensis, Paenibacillus septentrionalis, Paenibacillus sepulcri, Paenibacillus shenyangensis, Paenibacillus shirakamiensis, Paenibacillus siamensis, Paenibacillus silagei, Paenibacillus sinopodophylli, Paenibacillus solani, Paenibacillus soli, Paenibacillus sonchi, Paenibacillus sophorae, Paenibacillus sputi, Paenibacillus stellifer, Paenibacillus susongensis, Paenibacillus swuensis, Paenibacillus taichungensis, Paenibacillus taiwanensis, Paenibacillus tarimensis, Paenibacillus telluris, Paenibacillus terreus, Paenibacillus terrigena, Paenibacillus thailandensis, Paenibacillus thermophilus, Paenibacillus thiaminolyticus, Paenibacillus tianmuensis, Paenibacillus tibetensis, Paenibacillus timonensis, Paenibacillus tundrae, Paenibacillus turicensis, Paenibacillus typhae, Paenibacillus uliginis, Paenibacillus urinalis, Paenibacillus validus, Paenibacillus vini, Paenibacillus vulneris, Paenibacillus wenxiniae, Paenibacillus wooponensis, Paenibacillus woosongensis, Paenibacillus wulumuqiensis, Paenibacillus wynnii, Paenibacillus xanthinilyticus, Paenibacillus xinjiangensis, Paenibacillus xylanexedens, Paenibacillus xylanilyticus, Paenibacillus xylanisolvens, Paenibacillus yonginensis, Paenibacillus yunnanensis, Paenibacillus zanthoxyli, or Paenibacillus zeae.

In some aspects, the nucleic acid molecule is operatively linked to a promoter element that is heterologous to at least one of the second polynucleotide sequence and Paenibacillus.

In some aspects, the first polynucleotide sequence comprises: (a) a codon-optimized polynucleotide sequence having at least 50%, 60%, 70%, 80% or 90% sequence identity with SEQ ID NO: 1, 3, 5, 7 or 9, which is expressed at a higher rate or level in the Paenibacillus endospore compared to the respective original SEQ ID NO: 1, 3, 5, 7 or 9, under identical conditions.

In an alternative aspect, the disclosure provides a fusion protein comprising an N-terminal signal peptide operably linked to a polypeptide heterologous to the N-terminal signal peptide, wherein the N-terminal signal peptide comprises: (a) a polypeptide comprising an amino acid sequence having at least 50%, 60%, 70%, or 80% sequence identity with the amino acid sequence of SEQ ID NO: 2, 4, 6, 8 or 10; or (b) a polypeptide comprising a fragment of at least 5, 10, 15, 20, 25 or 30 consecutive amino acids of SEQ ID NO: 2, 4, 6, 8 or 10; wherein the N-terminal signal peptide is capable of targeting the fusion protein to the spore surface of a Paenibacillus endospore.

In some aspects, the polypeptide heterologous to the N-terminal signal peptide comprises: (a) at least one of a plant growth or immune stimulating protein; (b) an enzyme; (c) a polypeptide heterologous to Paenibacillus; (d) a therapeutic protein (e.g., an antibiotic or anti-inflammatory protein); or (e) a protein that provides an agriculturally-significant property, included, but not limited to: insecticidal/insectistatic activity, bactericidal/bacteriostatic activity, fungicidal/fungistatic activity, plant growth, health or immune-stimulating activity, and/or improved abiotic environmental resistance. Other agriculturally-significant properties include improved crop characteristics including: emergence, crop yields, protein content, oil content, starch content, more developed root system, improved root growth, improved root size maintenance, improved root effectiveness, improved stress tolerance (e.g., against drought, heat, salt, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

In some aspects, the fusion protein further comprises: (a) a polypeptide containing one or more protease cleavage sites, positioned between the N-terminal signal peptide and the polypeptide heterologous to the N-terminal signal peptide; (b) a polypeptide comprising a selectable marker (e.g., a protein that confers resistance to an antibiotic); (c) a polypeptide comprising a visualization element (e.g., a fluorescent tag such as GFP); (d) a polypeptide comprising at least one protein recognition/purification domain (e.g., a His-tag); or (e) a polypeptide comprising a flexible linker element, connecting the signal peptide and the polypeptide heterologous to the N-terminal signal peptide.

In an alternative aspect, the disclosure provides a recombinant Paenibacillus cell comprising a bacterial chromosome comprising the nucleic acid molecule of any one of the previous aspects.

In an alternative aspect, the disclosure provides a vector comprising the nucleic acid molecule of any one of the previous aspects, wherein the vector comprises a plasmid, an artificial chromosome, or a viral vector.

In some aspects, the vector further comprising at least one of the following: (a) an origin of replication that provides stable maintenance in a Paenibacillus cell; (b) an origin of replication that provides selectively non-stable maintenance in a Paenibacillus cell; (c) a temperature-sensitive origin of replication that provides selectively non-stable maintenance in a Paenibacillus cell; (d) a polynucleotide encoding a selection marker, operably linked to an expression control sequence; or (e) a polynucleotide encoding a plant growth stimulating protein, operably linked to an expression control sequence.

In alternative aspects, the disclosure provides a recombinant Paenibacillus cell transformed with a vector comprising the nucleic acid molecule of any one of the aspects disclosed herein.

In some aspects, the Paenibacillus cell is a Paenibacillus species, comprising: Paenibacillus sp. NRRL B-50972, Paenibacillus terrae, Paenibacillus polymyxa, or Paenibacillus peoriae. In other exemplary aspects, the Paenibacillus cell may be selected from any of the exemplary Paenibacillus species described herein.

In alternative aspects, the disclosure provides a method of displaying a heterologous fusion protein on the spore surface of a Paenibacillus endospore, the method comprising: a) transforming a Paenibacillus cell capable of sporulation with a recombinant vector comprising the nucleic acid molecule of any one of the aspects disclosed herein; and b) expressing the fusion protein encoded by the nucleic acid molecule of any one of the aspects disclosed herein under sporulation conditions such that the fusion protein is targeted to the spore surface of the Paenibacillus endospore resulting from the sporulation, wherein the N-terminal signal peptide comprises: (i) a polypeptide comprising an amino acid sequence having at least 50%, 60%, 70%, 80% or 90% sequence identity with the amino acid sequence of SEQ ID NO: 2; or (ii) a fragment of at least 5, 10, 15 or 20 consecutive amino acids from SEQ ID NO: 2.

In alternative aspects, the disclosure provides a composition comprising: a) one or more recombinant endospore-producing Paenibacillus cells that express the fusion protein of any one of the aspects disclosed herein, wherein the polypeptide heterologous to the N-terminal signal peptide comprises a plant growth or immune stimulating protein; and b) at least one biological control agent; optionally, in a synergistically effective amount.

In alternative aspects, the disclosure provides a seed treated with at least one of the nucleic acids, fusion proteins, bacterial cells or compositions of any one of the aspects disclosed herein.

In alternative aspects, the disclosure provides a method of treating a plant, a seed, a plant part, or the soil surrounding the plant to enhance plant growth and/or promote plant health comprising the step of simultaneously or sequentially applying: a) recombinant endospore-producing Paenibacillus endospores that express the fusion protein of any of the aspects disclosed herein, wherein the polypeptide heterologous to the N-terminal signal peptide comprises a plant growth or immune stimulating protein; and b) at least one biological control agent; optionally, in a synergistically effective amount.

In alternative aspects, the disclosure provides a method of screening a host plant treated with a recombinant Paenibacillus endospore, comprising the following steps: a) applying a composition comprising a Paenibacillus endospore modified to express a fusion protein according to any of the aspects disclosed herein, to a seed, a seedling, or a vegetative plant capable of being permanently or transiently colonized by a Paenibacillus, to produce a treated seed, seedling, or vegetative plant; b) screening the treated seed, seedling, or vegetative plant by detecting and optionally measuring a trait, component, or attribute of the treated seed, seedling, or vegetative plant.

In some aspects, the screening step comprises one or more of the following: a) at least one in vitro assay comprising detecting and optionally quantifying the presence, level, change in level, activity, or localization of one or more compounds contained in an extract prepared from a cell or tissue sample obtained from the treated seed, seedling, or vegetative plant; and/or b) at least one in vivo assay comprising detecting and optionally quantifying a trait, component, or attribute of the treated seed, seedling, or vegetative plant.

In alternative aspects, the disclosure provides a method of screening heterologous proteins or peptides expressed in a Paenibacillus cell for agriculturally-significant properties, comprising: a) modifying a Paenibacillus cell to express a fusion protein according to the aspects disclosed herein to produce a recombinant Paenibacillus cell; and b) screening the Paenibacillus cell by detecting and optionally quantifying a level or activity of a compound produced by the recombinant Paenibacillus cell.

In alternative aspects, the disclosure provides a method for identifying spore surface-targeting sequences in Paenibacillus and other bacterial genera suitable for endospore display, comprising: screening a genome of a Paenibacillus or another endospore-forming bacteria of interest for open reading frames that encode proteins having multiple collagen-like triplet repeats of “Gly-X-X” (“GXX repeats” where “X” represents any amino acid); and determining that the protein localizes to the spore surface by microscopy or experimentally. In some aspects, the protein localization is determined using transmission electron microscopy or mass spectrometry. In other aspects, the putative N-terminal targeting sequence from a protein that localizes to the spore surface is fused to a reporter gene and the resulting fusion protein is expressed in an endospore-forming bacterium. In yet other aspects, the resulting fusion protein is analyzed for expression on the surface of such endospore-forming bacterium. In another aspect, if such expression is detected, the reporter gene is replaced with a nucleotide sequence of interest and such second fusion protein is expressed in an endospore-forming bacterium.

In some aspects, the disclosure provides spore surface-targeting targeting sequences from Paenibacillus and other bacterial genera comprising an N-terminal targeting sequence of a protein identified via the aforementioned method. This N-terminal targeting sequence may comprise the first 5, 10, 15, 20, 25, 30, 35, 40, 50, 60, 70, 80, 90, 100, 110, or 120 amino acids of the protein, or a fragment or variant thereof. In some aspects, the N-terminal targeting sequence is a variant that is at least 60%, 70%, 80%, 90% or 95% identical to the endogenous N-terminal targeting sequence. Spore surface targeting sequences in Paenibacillus and other bacterial genera identified using these methods may be used to generate heterologous fusion proteins according to any of the various embodiments described herein.

In selected aspects, the composition has been heat-inactivated or sterilized such that no viable Paenibacillus cells remain.

In alternative aspects, the disclosure provides a composition comprising an isolated and/or purified fusion protein according to any one of the aspects disclosed herein.

In alternative aspects, the disclosure provides a method of delivering a protein of interest to a plant, seed or field, comprising: applying a composition comprising a recombinant Paenibacillus endospore to a plant, seed, or field; wherein the recombinant Paenibacillus endospore has been modified to express a fusion protein according to any of the aspects disclosed herein.

In some aspects, the composition is applied to a field: a) pre- or post-planting; b) pre- or post-emergence; c) as a powder, suspension or solution; or d) wherein the composition further comprises one or more additional compounds that stimulate plant growth.

In some embodiments, the present invention provides a nucleic acid molecule encoding a fusion protein, comprising (a) a first polynucleotide sequence encoding an N-terminal signal peptide, operably linked to (b) a second polynucleotide sequence encoding a polypeptide heterologous to the N-terminal signal peptide, wherein the first polynucleotide sequence comprises: (i) a polynucleotide sequence comprising at least 15, 30, 45, 60, 75 or 90 nucleotides; (ii) a polynucleotide sequence having at least 50%, 60%, 70%, 80% or 90% sequence identity with SEQ ID NO: 1, 3, 5, 7, 9, 19, 23, 25, 27, or 29; or (iii) a polynucleotide sequence comprising a fragment of at least 45, 90, 135, 180, 225, 270, 315, or 345 consecutive nucleotides of SEQ ID NO: 1, 3, 5, 7, 9, 19, 23, 25, 27, or 29; wherein the N-terminal signal peptide is capable of targeting the fusion protein to a spore surface of a Paenibacillus endospore.

In one aspect, the fragment starts at the first nucleotide of SEQ ID NO: 1, 3, 5, 7, 9, 19, 23, 25, 27, or 29. In another aspect, the first polynucleotide sequence comprises a polynucleotide sequence having at least 50%, 60%, 70%, 80% or 90% sequence identity with SEQ ID NO: 1, 7, 19, or 27. In another aspect, the fragment encodes amino acids 1-15, or 1-30, 1-45, 1-60, 1-75, 1-90, 1-105, or 1-115 of SEQ ID NO: 2 or SEQ ID NO: 8.

In one embodiment, the polypeptide heterologous to the N-terminal signal peptide comprises: (a) a plant growth-stimulating protein; (b) an enzyme; (c) a protein; (d) a polypeptide heterologous to Paenibacillus; (e) a therapeutic protein; or (f) a plant immune-stimulating protein.

In another embodiment, the nucleic acid further comprising a third polynucleotide sequence, encoding: (a) a polypeptide comprising one or more protease cleavage sites, wherein the polypeptide is positioned between the N-terminal signal peptide and the polypeptide heterologous to the N-terminal signal peptide; (b) a polypeptide comprising a selectable marker; (c) a polypeptide comprising a visualization marker; (d) a polypeptide comprising a protein recognition/purification domain; or (e) a polypeptide comprising a flexible linker element, which connects the N-terminal signal peptide and the polypeptide heterologous to the N-terminal signal peptide.

In yet another embodiment, the Paenibacillus endospore is an endospore formed by a Paenibacillus species, comprising: Paenibacillus sp. NRRL B-50972, Paenibacillus terrae, Paenibacillus polymyxa, or Paenibacillus peoriae; or an endospore formed by a bacterium that possesses a 16S rRNA gene that shares at least 97, 98 or 99% identity with a 16S rRNA gene of a Paenibacillus species.

In one aspect, the nucleic acid molecule is operatively linked to a promoter element that is heterologous to at least one of the second polynucleotide sequences and Paenibacillus.

In another aspect, the first polynucleotide sequence comprises: a codon-optimized polynucleotide sequence having at least 50%, 60%, 70%, 80% or 90% sequence identity with SEQ ID NO: 1, 3, 5, 7, 9, 19, 23, 25, 27, or 29; or a fragment thereof, which is expressed at a higher rate or level in the Paenibacillus endospore compared to the corresponding unoptimized sequence under identical conditions.

In yet another aspect, the present invention relates to a fusion protein comprising an N-terminal signal peptide operably linked to a polypeptide heterologous to the N-terminal signal peptide, wherein the N-terminal signal peptide comprises: (i) a polypeptide comprising at least 15, 30, 45, 60, 75, 90, 105, or 115 residues; (ii) a polypeptide comprising an amino acid sequence having at least 50%, 60%, 70%, 80%, or 90% sequence identity with the amino acid sequence of SEQ ID NO: 2, 4, 6, 8, 10, 18, 20, 21, 22, 24, 26, 28, 30, 31, or 32; or (iii) a polypeptide comprising a fragment of at least 15, 30, 45, 60, 75, 90, 105, or 115 consecutive amino acids of SEQ ID NO: 2, 4, 6, 8, 10, 18, 20, 21, 22, 24, 26, 28, 30, 31, or 32; wherein the N-terminal signal peptide is capable of targeting the fusion protein to the spore surface of a Paenibacillus endospore.

In one embodiment, the fragment starts at the first amino acid of SEQ ID NO: 2, 4, 6, 8, 10, 18, 20, 21, 22, 24, 26, 28, 30, 31, or 32. In another embodiment, the polypeptide sequence comprises a sequence having at least 50%, 60%, 70%, 80% or 90% sequence identity with SEQ ID NO: 2, 8, 20, 21, 22, 28, 31, or 32. In yet another embodiment, the fragment comprises amino acids 1-15, or 1-30, 1-45, 1-60, 1-75, 1-90, 1-105, or 1-115 of SEQ ID NO: 2, 8, 31, or 32.

In one embodiment, the fragment starts at the 80th amino acid of SEQ ID NO: 2, 4, 6, 8, 10, 18, 20, 21, 22, 24, 26, 28, 30, 31, or 32. In yet another embodiment, the fragment comprises amino acids 80-100, 85-100, 90-100, 91-100, 92-100, 93-100, 94-100, 90-99, 90-98, 90-97, 90-96, 91-98, 92-98, 93-98, 91-97 or 91-96 of SEQ ID NO: 2, 8, 31, or 32. In yet another embodiment the fragment consists of or consists essentially of amino acids 91-98, 90-100, 85-95, 80-100 or any contiguous portion of amino acids 80-100 that still contains amino acids 91-98 of SEQ ID NO: 2. In another aspect, such fragment in this embodiment is linked to other amino acids, which have a sequence identity of less than 50%, less than 60%, less than 70%, or less than 80% to the portion of SEQ ID NO: 2 that does not contain the fragment. In this aspect, the polypeptide sequence of 1-120 containing the fragment, wherein the fragment is assigned the amino acid residue numbers above (e.g., 91-98, 90-100, 85-95, or 80-100) has less than 50%, 60%, 70%, or 80% sequence identity to SEQ ID NO:2 as to the amino acids in the polypeptide sequence (of 1-120) other than the fragment.

In some aspects, the polypeptide heterologous to the N-terminal signal peptide comprises: (a) a plant growth-stimulating protein; (b) an enzyme; (c) a protein; (d) a polypeptide heterologous to Paenibacillus; (e) a therapeutic protein; or (f) a plant immune-stimulating protein.

In other aspects, the fusion protein further comprises: (a) a polypeptide containing one or more protease cleavage sites, positioned between the N-terminal signal peptide and the polypeptide heterologous to the N-terminal signal peptide; (b) a polypeptide comprising a selectable marker; (c) a polypeptide comprising a visualization marker; (d) a polypeptide comprising at least one protein recognition/purification domain; or (e) a polypeptide comprising a flexible linker element, connecting the signal peptide and the polypeptide heterologous to the N-terminal signal peptide.

In some embodiments, the present invention provides a recombinant Paenibacillus cell comprising a bacterial chromosome comprising a nucleic acid molecule disclosed herein.

In other embodiments, the present invention relates to a vector comprising a nucleic acid molecule disclosed herein, wherein the vector comprises a plasmid, an artificial chromosome, or a viral vector. In one aspect, the vector further comprises at least one of the following: (a) an origin of replication that provides stable maintenance in a Paenibacillus cell; (b) an origin of replication that provides selectively non-stable maintenance in a Paenibacillus cell; (c) a temperature-sensitive origin of replication that provides selectively non-stable maintenance in a Paenibacillus cell; (d) a polynucleotide encoding a selection marker, operably linked to an expression control sequence; or (e) a polynucleotide encoding a plant growth stimulating protein, operably linked to an expression control sequence.

In yet other embodiments, the present invention provides a recombinant Paenibacillus cell transformed with a vector comprising a nucleic acid molecule disclosed herein. In one aspect, the recombinant Paenibacillus cell is a Paenibacillus species, comprising: Paenibacillus sp. NRRL B-50972, Paenibacillus terrae, Paenibacillus polymyxa, or Paenibacillus peoriae; or a bacterium that possesses a 16S rRNA gene that shares at least 97, 98 or 99% identity with a 16S rRNA gene of a Paenibacillus species.

In some aspects, the present invention provides a method of displaying a heterologous fusion protein on a spore surface of a Paenibacillus endospore, the method comprising: a) transforming a Paenibacillus cell capable of sporulation with a recombinant vector comprising a nucleic acid molecule disclosed herein; and b) expressing the fusion protein encoded by a nucleic acid molecule disclosed herein under sporulation conditions such that the fusion protein is targeted to the spore surface of the Paenibacillus endospore resulting from the sporulation, wherein the N-terminal signal peptide comprises: (i) a polypeptide comprising at least 5, 10, 15, 20, 25 or 30 residues; (ii) a polypeptide comprising an amino acid sequence having at least 50%, 60%, 70%, 80% or 90% sequence identity with the amino acid sequence of SEQ ID NO: 2, 4, 6, 8 or 10; or (iii) a fragment of at least 5, 10, 15, 20, 25 or 30 consecutive amino acids of SEQ ID NO: 2, 4, 6, 8 or 10.

In one embodiment, the present invention relates to a composition comprising: a) one or more recombinant endospore-producing Paenibacillus cells that express a fusion protein disclosed herein, wherein the polypeptide heterologous to the N-terminal signal peptide comprises a plant growth or immune stimulating protein; and b) at least one biological control agent; optionally, in a synergistically effective amount.

In yet another embodiment, the present invention provides a seed treated with a nucleic acid disclosed herein, a fusion protein disclosed herein, a recombinant bacterial cell disclosed herein, or a composition disclosed herein.

In one aspect, the present invention provides a method of treating a plant, a seed, a plant part, or the soil surrounding the plant to enhance plant growth and/or promote plant health comprising the step of simultaneously or sequentially applying: a) recombinant endospore-producing Paenibacillus endospores that express a fusion protein disclosed herein, wherein the polypeptide heterologous to the N-terminal signal peptide comprises a plant growth or immune stimulating protein; and b) at least one biological control agent; optionally, in a synergistically effective amount.

In another aspect, the present invention relates to a method of screening a host plant treated with a recombinant Paenibacillus endospore, comprising the following steps: a) applying a composition comprising a Paenibacillus endospore modified to express a fusion protein disclosed herein, to a seed, a seedling, or a vegetative plant capable of being permanently or transiently colonized by a Paenibacillus, to produce a treated seed, seedling, or vegetative plant; b) screening the treated seed, seedling, or vegetative plant by detecting and optionally measuring a trait, component, or attribute of the treated seed, seedling, or vegetative plant.

In some embodiments, the screening step comprises one or more of the following: a) at least one in vitro assay comprising detecting and optionally quantifying the presence, level, change in level, activity, or localization of one or more compounds contained in an extract prepared from a cell or tissue sample obtained from the treated seed, seedling, or vegetative plant; and/or b) at least one in vivo assay comprising detecting and optionally quantifying a trait, component, or attribute of the treated seed, seedling, or vegetative plant.

In one aspect, the present invention relates to a method of screening heterologous proteins or peptides expressed in a Paenibacillus cell for agriculturally-significant properties, comprising: a) modifying a Paenibacillus cell to express a fusion protein disclosed herein to produce a recombinant Paenibacillus cell; and b) screening the Paenibacillus cell by detecting and optionally quantifying a level or activity of a compound produced by the recombinant Paenibacillus cell.

Also provided is a method of treating a plant, a seed, a human, or an animal, comprising: administering to the plant, seed, human, or animal a composition comprising an endospore produced by a recombinant Paenibacillus cell; wherein the recombinant Paenibacillus cell expresses a fusion protein disclosed herein.

In some aspects, the composition has been heat-inactivated or sterilized such that no viable Paenibacillus cells remain.

In other aspects, the present invention relates to a composition comprising an isolated and/or purified fusion protein as disclosed herein.

In one embodiment, the present invention provides a method of delivering a protein of interest to a plant, seed or field, comprising: applying a composition comprising a recombinant Paenibacillus endospore to a plant, seed, or field; wherein the recombinant Paenibacillus endospore has been modified to express a fusion protein disclosed herein.

In certain aspects, the composition is applied to a field: a) pre- or post-planting; b) pre- or post-emergence; c) as a powder, suspension or solution; and/or d) wherein the composition further comprises one or more additional compounds that stimulate plant growth or protect plants from pests.

In a particular embodiment, the present invention relates to a method for identifying an N-terminal signal sequence that is capable of targeting a protein to a spore surface of an endospore-forming bacterium, comprising: screening a genome of the endospore-forming bacterium for at least one open reading frame which encodes a protein having multiple collagen-like triplet repeats having the sequence “GLY-X-X,” wherein “X” represents “any amino acid”; and determining that at least one of the proteins identified in the screening step localizes to the spore surface of the endospore-forming bacterium by microscopy or experimentally.

In one aspect, the endospore-forming bacterium includes a hair-like structure that is proteolytically resistant. In another aspect, the method further comprises identifying a putative N-terminal signal sequence from at least one protein identified in the determining step as localizing to the spore surface and expressing in the endospore-forming bacterium a fusion protein comprising the putative N-terminal signal sequence and a reporter gene.

In another aspect, the method further comprises selecting the fusion protein based on expression of the fusion protein on the spore surface. In yet another aspect, the method further comprises replacing the reporter gene in the fusion protein that is selected with a nucleotide sequence of interest to create a second fusion protein and expressing the second fusion protein in the endospore-forming bacterium.

In some embodiments, the bacterium is a member of the genus Paenibacillus, Viridibacillus, Brevibacillus or Lysinibacillus. In one embodiment, the bacterium is a member of the genus Paenibacillus.

In some aspects, localization is determined using transmission electron microscopy or mass spectrometry.

In other aspects, the present invention relates to a nucleic acid molecule encoding an N-terminal signal peptide, wherein the signal peptide comprises: a) a contiguous segment of at least 5, 10, 15, 20, 25, 30, 35, 40, 45, 50, 60, 70, 80, 90, 100, 110, or 120 N-terminal residues of a protein determined to localize to a spore surface of an endospore-forming bacterium by a method disclosed herein; b) a sequence having at least 60%, 65%, 70%, 75%, 80%, 85%, 90%, or 95% sequence identity to a contiguous segment of at least 5, 10, 15, 20, 25, 30, 35, 40, 45, 50, 60, 70, 80, 90, 100, 110, or 120 N-terminal residues of a protein determined to localize to a spore surface of an endospore-forming bacterium by a method disclosed herein; wherein the segment or sequence is capable of targeting a fusion protein comprising the segment or sequence to the spore surface of an endospore-forming bacterium when expressed in the bacterium.

In yet other aspects, the present invention provides a nucleic acid molecule encoding a fusion protein, comprising (a) a first polynucleotide sequence encoding an N-terminal signal peptide, operably linked to (b) a second polynucleotide sequence encoding a polypeptide heterologous to the N-terminal signal peptide, wherein the first polynucleotide sequence comprises the sequence or segment disclosed herein and the N-terminal signal peptide is capable of targeting the fusion protein to the spore surface of a bacterial endospore.

In one aspect, the nucleic acid molecule further comprises an upstream regulatory sequence that causes transcription of the fusion protein during sporulation of a bacterial cell. In one embodiment, the bacterial cell is a Paenibacillus family member.

In some embodiments, the upstream regulatory sequence comprises: (a) any of SEQ ID NOs: 11-15; (b) a sequence comprising a fragment of at least 25, 50, 100, 150 contiguous nucleotides of any of SEQ ID NOs: 11-15; (c) a sequence having at least 60%, 70%, 80%, or 90% sequence identity compared to any of SEQ ID NOs: 11-15, or to a 25, 50, 100 or 150 nucleotide fragment thereof; wherein the upstream regulatory sequence comprises a promoter that is transcriptionally active during sporulation of the bacterial cell.

In yet other embodiments, the present invention provides a nucleic acid molecule encoding an upstream regulatory sequence and a protein of interest, comprising: (a) a first polynucleotide sequence encoding the upstream regulatory sequence, operably linked to (b) a second polynucleotide sequence encoding the protein of interest; wherein the protein of interest is heterologous to the upstream regulatory sequence and the upstream regulatory sequence causes transcription of the protein of interest during sporulation of a bacterial cell. In one aspect, the bacterial cell is a Paenibacillus family member.

In another aspect, the upstream regulatory sequence comprises: (a) any of SEQ ID NOs: 11-15; (b) a sequence comprising a fragment of at least 25, 50, 100, 150 contiguous nucleotides of any of SEQ ID NOs: 11-15; (c) a sequence having at least 60%, 70%, 80%, or 90% sequence identity compared to any of SEQ ID NOs: 11-15, or to a 25, 50, 100 or 150 nucleotide fragment thereof; wherein the upstream regulatory sequence comprises a promoter that is transcriptionally active during sporulation of the bacterial cell.

### BRIEF DESCRIPTION OF THE DRAWINGS

FIG. 1 depicts a transmission electron micrograph of a Paenibacillus sp. NRRL B-50972 endospore. Hair-like structures comprised of collagen-like protein are shown extending from the endospore surface and one such structure is denoted by an arrow.

FIG. 2A depicts phase contrast (left) and epifluorescent (right) micrographs (1000× magnification) of a Paenibacillus sp. NRRL B-50972 endospore expressing an exemplary N-terminal targeting sequence according to the disclosure, specifically a (SEQ ID NO: 2)-GFP fusion protein construct which is localized to the endospore surface as shown by this figure. The fluorescence produced by the GFP protein in the right panel corresponds with the image of the cell observed with phase contrast microscopoy in the left panel indicating correct localization of the GFP to the endospore surface.

FIG. 2B depicts a flow cytometry histogram of Paenibacillus sp. NRRL B-50972 endospores expressing an exemplary N-terminal targeting sequence according to the disclosure, specifically a (SEQ ID NO: 2)-GFP fusion protein construct, which is localized to the endospore surface (shaded area). Wild-type Paenibacillus sp. NRRL B-50972 endospores with no observable GFP fluorescence are shown for comparison (open, dotted line area). 10,000 events are shown for each spore population on this figure.

FIG. 3 depicts a local sequence alignment of the N-terminal portion of SEQ ID NO: 2 and SEQ ID NO: 8, which are exemplary spore surface-targeting sequences according to the disclosure. A consensus sequence (SEQ ID NO: 32) is shown below the alignment.

FIG. 4 depicts a multiple sequence alignment showing SEQ ID NO: 2 and the N-terminal collagen-like repeat domains of putative homologs expressed by Paenibacillus strains. A consensus sequence and a minimal N-terminal targeting domain for spore surface-targeting are annotated on this figure.

### DETAILED DESCRIPTION

The disclosure provides genetic constructs capable of targeting a fusion protein to a Paenibacillus spore surface, as well as compositions and methods that use these constructs to deliver heterologous molecules of interest (e.g., peptides, proteins) to various environments, such as plants. For example, following treatment with the recombinant Paenibacillus endospores, treated plants may be screened to detect changes attributable to the heterologous protein delivered via the Paenibacillus endospores. Such changes may include alterations in the host plant's growth rate or yield; enhanced plant health (e.g., resistance to environmental stress, disease or pests); and the display of enhanced, modified or otherwise new attributes, compared to host plants grown under the same conditions absent treatment with the recombinant Paenibacillus endospores. The use of a targeting sequence that efficiently targets the heterologous protein to the spore surface also provides a platform for high-throughput screening for useful heterologous proteins that, for example, are capable of enhancing, modifying, and/or conferring new plant traits or attributes.

The canonical spore formation process (elucidated based on studies using B. subtilis) involves asymmetric cell division of a vegetative cell to form a mother cell and a forespore, which develop as two distinct compartments separated by an intervening septum. Eventually, the peptidoglycan in the septum is degraded and the forespore is engulfed by the mother cell, forming a cell within a cell. Intercellular communication between the mother cell and forespore coordinates cell-specific gene expression in each cell, resulting in the production of endospore-specific compounds, formation of a cortex layer around the forespore and deposition of the coat.

In some Bacillus species, e.g., B. subtilis, B. licheniformis, and B. pumilus, this coat will go on to become the outermost layer of the endospore. The forespore undergoes a final dehydration and maturation into a complete endospore. The mother cell is subsequently degraded via programmed cell death, resulting in a release of the endospore into the environment. The endospore will then typically remain in a dormant state until more favorable conditions or particular stimuli trigger germination and a return to the vegetative state.

As the outermost surface between the spore and the environment, the coat layer serves many critical functions. In particular, this layer acts as a semipermeable barrier to environmental insults and mediates interactions with the surrounding environment, and thus plays an important role in maintaining the viability of the spore and in the sensing of conditions that trigger germination of the endospore. The coat layer is also a target of clinical research as it contains cell surface molecules in pathogenic strains of bacteria that contribute to host immune cell recognition. Methods of displaying heterologous proteins on the spore coat of B. subtilis have been developed using fusion protein constructs containing a B. subtilis spore coat protein such as CotC fused to a protein of interest. However, the spore surface proteins of Paenibacillus are unknown and thus studies using B. subtilis fail to provide guidance as to how fusion proteins may be targeted to the spore surface of other bacterial genera, such as Paenibacillus.

In contrast, the disclosure provides N-terminal constructs and fusion proteins comprising the same that are capable of targeting fusion protein constructs to the spore surface of Paenibacillus cells. The N-terminal signal sequence used to target the fusion protein to the spore surface may comprise a polypeptide having a sequence as represented by SEQ ID NO: 2, 4, 6, 8 or 10. Alternatively, in select embodiments, this N-terminal signal sequence may comprise a fragment or variant of SEQ ID NO: 2, 4, 6, 8 or 10 sufficient to retain the spore surface targeting functionality. For example, a fragment may comprise the first 10, 15, 20, 25 or 30 amino acids of SEQ ID NO: 2, 4, 6, 8 or 10. Further alternative aspects include N-terminal signal sequences encoded by a nucleic acid comprising the nucleotide sequence of SEQ ID NO: 1, 3, 5, 7 or 9, or a fragment or variant thereof. These and other embodiments are described herein.

Throughout the disclosure, the term “comprise” or any derivative thereof (e.g., comprising, comprises) may be replaced with “consist essentially of”, “consist of”, or the applicable corresponding derivative thereof.

As used herein, “Paenibacillus” refers to endospore-producing bacteria classified in the Paenibacillus genus. This term encompasses, without limitation, various Paenibacillus family members including Paenibacillus sp. NRRL B-50972, Paenibacillus abyssi, Paenibacillus aceti, Paenibacillus aestuarii, Paenibacillus agarexedens, Paenibacillus agaridevorans, Paenibacillus alginolyticus, Paenibacillus algorifonticola, Paenibacillus alkaliterrae, Paenibacillus alvei, Paenibacillus amylolyticus, Paenibacillus anaericanus, Paenibacillus antarcticus, Paenibacillus apiarius, Paenibacillus arachidis, Paenibacillus assamensis, Paenibacillus azoreducens, Paenibacillus azotofixans, Paenibacillus baekrokdamisoli, Paenibacillus barcinonensis, Paenibacillus barengoltzii, Paenibacillus borealis, Paenibacillus bovis, Paenibacillus brasilensis, Paenibacillus camelliae, Paenibacillus campinasensis, Paenibacillus castaneae, Paenibacillus catalpae, Paenibacillus cathormii, Paenibacillus cavernae, Paenibacillus cellulosilyticus, Paenibacillus cellulositrophicus, Paenibacillus chartarius, Paenibacillus chibensis, Paenibacillus chinjuensis, Paenibacillus chitinolyticus, Paenibacillus chondroitinus, Paenibacillus chungangensis, Paenibacillus cineris, Paenibacillus cisolokensis, Paenibacillus contaminans, Paenibacillus cookii, Paenibacillus cucumis, Paenibacillus curdlanolyticus, Paenibacillus daejeonensis, Paenibacillus darwinianus, Paenibacillus dauci, Paenibacillus dendritiformis, Paenibacillus dongdonensis, Paenibacillus doosanensis, Paenibacillus durus, Paenibacillus edaphicus, Paenibacillus ehimensis, Paenibacillus elgii, Paenibacillus endophyticus, Paenibacillus etheri, Paenibacillus faecis, Paenibacillus favisporus, Paenibacillus ferrarius, Paenibacillus filicis, Paenibacillus fonticola, Paenibacillus forsythias, Paenibacillus frigoriresistens, Paenibacillus gansuensis, Paenibacillus gelatinilyticus, Paenibacillus ginsengarvi, Paenibacillus ginsengihumi, Paenibacillus ginsengisoli, Paenibacillus glacialis, Paenibacillus glucanolyticus, Paenibacillus glycanilyticus, Paenibacillus gordonae, Paenibacillus graminis, Paenibacillus granivorans, Paenibacillus guangzhouensis, Paenibacillus harenae, Paenibacillus hemerocallicola, Paenibacillus hispanicus, Paenibacillus hodogayensis, Paenibacillus hordei, Paenibacillus humicus, Paenibacillus hunanensis, Paenibacillus illinoisensis, Paenibacillus jamilae, Paenibacillus jilunlii, Paenibacillus kobensis, Paenibacillus koleovorans, Paenibacillus konsidensis, Paenibacillus koreensis, Paenibacillus kribbensis, Paenibacillus kyungheensis, Paenibacillus lactis, Paenibacillus larvae, Paenibacillus larvae, Paenibacillus larvae, Paenibacillus lautus, Paenibacillus lemnae, Paenibacillus lentimorbus, Paenibacillus lentus, Paenibacillus liaoningensis, Paenibacillus lupini, Paenibacillus macerans, Paenibacillus macquariensis, Paenibacillus macquariensis, Paenibacillus macquariensis, Paenibacillus marchantiophytorum, Paenibacillus marinisediminis, Paenibacillus massiliensis, Paenibacillus medicaginis, Paenibacillus mendelii, Paenibacillus methanolicus, Paenibacillus montaniterrae, Paenibacillus motobuensis, Paenibacillus mucilaginosus, Paenibacillus nanensis, Paenibacillus naphthalenovorans, Paenibacillus nasutitermitis, Paenibacillus nematophilus, Paenibacillus nicotianae, Paenibacillus oceanisediminis, Paenibacillus odorifer, Paenibacillus oenotherae, Paenibacillus oryzae, Paenibacillus pabuli, Paenibacillus panacisoli, Paenibacillus panaciterrae, Paenibacillus pasadenensis, Paenibacillus pectinilyticus, Paenibacillus peoriae, Paenibacillus periandrae, Paenibacillus phoenicis, Paenibacillus phyllosphaerae, Paenibacillus physcomitrellae, Paenibacillus pini, Paenibacillus pinihumi, Paenibacillus pinesoli, Paenibacillus pocheonensis, Paenibacillus polymyxa, Paenibacillus popilliae, Paenibacillus populi, Paenibacillus prosopidis, Paenibacillus provencensis, Paenibacillus pueri, Paenibacillus puldeungensis, Paenibacillus pulvifaciens, Paenibacillus purispatii, Paenibacillus qingshengii, Paenibacillus quercus, Paenibacillus radicis, Paenibacillus relictisesami, Paenibacillus residui, Paenibacillus rhizoryzae, Paenibacillus rhizosphaerae, Paenibacillus rigui, Paenibacillus riograndensis, Paenibacillus ripae, Paenibacillus sabinae, Paenibacillus sacheonensis, Paenibacillus salinicaeni, Paenibacillus sanguinis, Paenibacillus sediminis, Paenibacillus segetis, Paenibacillus selenii, Paenibacillus selenitireducens, Paenibacillus senegalensis, Paenibacillus septentrionalis, Paenibacillus sepulcri, Paenibacillus shenyangensis, Paenibacillus shirakamiensis, Paenibacillus siamensis, Paenibacillus silagei, Paenibacillus sinopodophylli, Paenibacillus solani, Paenibacillus soli, Paenibacillus sonchi, Paenibacillus sophorae, Paenibacillus sputi, Paenibacillus stellifer, Paenibacillus susongensis, Paenibacillus swuensis, Paenibacillus taichungensis, Paenibacillus taiwanensis, Paenibacillus tarimensis, Paenibacillus telluris, Paenibacillus terrae, Paenibacillus terreus, Paenibacillus terrigena, Paenibacillus thailandensis, Paenibacillus thermophilus, Paenibacillus thiaminolyticus, Paenibacillus tianmuensis, Paenibacillus tibetensis, Paenibacillus timonensis, Paenibacillus tundrae, Paenibacillus turicensis, Paenibacillus typhae, Paenibacillus uliginis, Paenibacillus urinalis, Paenibacillus validus, Paenibacillus vini, Paenibacillus vulneris, Paenibacillus wenxiniae, Paenibacillus wooponensis, Paenibacillus woosongensis, Paenibacillus wulumuqiensis, Paenibacillus wynnii, Paenibacillus xanthinilyticus, Paenibacillus xinjiangensis, Paenibacillus xylanexedens, Paenibacillus xylanilyticus, Paenibacillus xylanisolvens, Paenibacillus yonginensis, Paenibacillus yunnanensis, Paenibacillus zanthoxyli, and Paenibacillus zeae.

In certain aspects, the Paenibacillus member used to express the fusion protein is Paenibacillus sp. NRRL B-50972, a Gram-positive, aerobic, and spore-forming bacterium isolated from soil. A sample of Paenibacillus sp. NRRL B-50972 has been deposited with the Agricultural Research Service Culture Collection located at the National Center for Agricultural Utilization Research, Agricultural Research Service, U.S. Department of Agriculture (NRRL), 1815 North University Street, Peoria, Ill. 61604, U.S.A., under the Budapest Treaty on Aug. 28, 2014. Given the general lack of knowledge about the basic composition or structure of the Paenibacillus spore, little is known about the process by which proteins are targeted to the spore surface during formation of this layer.

In certain aspects, the Paenibacillus member used to express the fusion protein is a bacterium that possesses a 16S rRNA gene that shares at least 97, 98 or 99 percent identity with a 16S rRNA gene of Paenibacillus sp. NRRL B-50972 or any of the other exemplary Paenibacillus family members disclosed herein. Alternatively, the Paenibacillus member used to express the fusion protein is a bacterium that possesses a DNA-DNA hybridization value of at least 70 percent to that of Paenibacillus sp. NRRL B-50972 or any of the other exemplary Paenibacillus family members disclosed herein. In another instance, the Paenibacillus member used to express the fusion protein is a bacterium that possesses an average nucleotide identity of 95, 96, 97, 98, or 99 percent to that of Paenibacillus sp. NRRL B-50972 or any of the other exemplary Paenibacillus family members disclosed herein.

The term “N-terminal signal sequence” generally refers to a polypeptide sequence located at or proximal to the amino terminus of a polypeptide, which directs localization of the polypeptide to a subcellular compartment, or for secretion. It is recognized and understood that this term may be used interchangeably with the terms “N-terminal targeting sequence,” “targeting sequence,” “signal sequence,” and “signal peptide,” depending on context. The N-terminal signal sequence may be retained as part of the polypeptide sequence of a mature protein or alternatively cleaved during or after the localization process. This term may be used to specifically refer to a polypeptide sequence located at or proximal to the amino terminus of a polypeptide, which directs localization of the polypeptide to the spore surface of a Paenibacillus endospore. In this context, the only required functionality of the N-terminal signal sequence is the capability to target the polypeptide of which it is a part to the spore surface of a Paenibacillus endospore.

A “plant” or “host plant,” includes any plant that possesses a rhizosphere or phyllosphere which Paenibacillus can colonize, as well as plants that can serve as a transient hosts for Paenibacillus bacteria. Colonization is not a requirement for the methods described herein and compositions to function, though it may be preferred in certain aspects of the disclosure.

As used herein, “biological control” is defined as control of a pathogen and/or insect and/or an acarid and/or a nematode by the use of a second organism or a biological molecule. Known mechanisms of biological control include bacteria that control root rot by out-competing fungi for space or nutrients on the surface of the root. Bacterial toxins, such as antibiotics, have been used to control pathogens. The toxin can be isolated and applied directly to the plant or the bacterial species may be administered so it produces the toxin in situ. Other means of exerting biological control include the application of certain fungi producing ingredients active against a target phytopathogen, insect, mite or nematode, or attacking the target pest/pathogen. “Biological control” may also encompass microorganisms having a beneficial effect on plant health, growth, vigor, stress response or yield. Application routes include spray application, soil application and seed treatment.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner The complex may comprise two strands forming a duplex structure, three or more strands forming a multi-stranded complex, a single self-hybridizing strand, or any combination of these. Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

As used herein, the term “sequence identity” refers to the degree to which two polynucleotide or amino acid sequences are identical (i.e., on a nucleotide-by-nucleotide or residue-by-residue basis, respectively) over the window of comparison. The percentage of sequence identity is calculated by comparing two optimally aligned sequences over the window of comparison, determining the number of positions at which the identical nucleic acid base (e.g., A, T, C, G for a polynucleotide sequence) occurs in both sequences to yield the number of matched positions, dividing the number of matched positions by the total number of positions in the window of comparison (i.e., the window size), and multiplying the result by 100 to yield the percentage of sequence identity. An equivalent calculation can be performed by comparing two aligned amino acid sequences.

With respect to the comparison of amino acid sequences, in addition to the measurement of sequence identity, a comparison may also take into account whether residue changes constitute “conservative” substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine

### N-Terminal Targeting Sequences

The disclosure provides N-terminal targeting sequences from Paenibacillus bacteria. Under stressful environmental conditions, Paenibacillus family bacteria undergo sporulation and form endospores that can stay dormant for extended periods of time. As described in detail herein, the outermost layer of Paenibacillus endospores is known as the spore surface and comprises a protein layer. Despite the growing body of literature available regarding Bacillus spore surface targeting sequences, there are no reported studies identifying homologous N-terminal targeting sequences in Paenibacillus. A bioinformatics analysis of the known spore surface-targeted proteins CotC, Bc1A, Bc1B or BetA fails to reveal any homologous N-terminal targeting sequences in Paenibacillus, suggesting that the spore surface targeting sequences of these proteins is not conserved in the Paenibacillus genus. Given the limited characterization of proteins that form and localize to the spore surface of Paenibacillus, one cannot easily deduce the N-terminal signal sequences necessary to target proteins to the spore surface in Paenibacillus generally or in particular species within this genus (e.g., Paenibacillus sp. NRRL B-50972).

Despite this lack of guidance in the available literature, the inventors have identified N-terminal targeting sequences capable of directing endogenous and fusion proteins to the spore surface of Paenibacillus cells.

For ease of reference, the SEQ ID NOs. for the nucleotide and polypeptide sequences referred to herein are listed in Table 1 below.

TABLE 1
Exemplary Paenibacillus N-Terminal Targeting Sequences
(i.e., SEQ ID NOs: 1-10, 18, 33, 34, 39-42, 45 and 49) and
Upstream Regulatory Sequences (i.e., SEQ ID NOs: 11-15).

| Sequence | |
|---|---|
| Identifier | Sequence |
| SEQ ID NO: 1 | ATGGTAGTATTATCTACTGGACCTATTGCAAACGATCCTGTTCTAGGAGTCAGACCC |
| | ACCCAACTGGTCACAGTAAAAATAGATAACCGAGATTCTGTAAATTCTTCTATCGTT |
| | TTGATCGAGGGTTTTATTTTAAACGGTAGCAGAACATTATATGTACAACAATTAGTG |
| | GTAGTGGGACCAAATGCGGTTATAACGAGGAATTTCTTTGCAAATGTAGACGCATTT |
| | GAATTCGTTTTTACCACTAGCGGACCAGCAGAGAATGAAACTCAAATTTCTGTTTGG |
| | GGTAAAGATGCATTGGGGCAATTAGTACCTGCCCATCGGTTAGTATCTGACGAACTT |
| | TTAGGAACCGATCGA |
| SEQ ID NO: 2 | MVVLSTGPIANDPVLGVRPTQLVTVKIDNRDSVNSSIVLIEGFILNGSRTLYVQQLV |
| | VVGPNAVITRNFFANVDAFEFVFTTSGPAENETQISVWGKDALGQLVPAHRLVSDEL |
| | LGTDR |
| SEQ ID NO: 3 | ATGCCTGCCTTGGATGAATGGAGTAGTATACAACAAATCGATATGGAGGTGTTTGTA |
| | TTGGGTCGTCCCGAATTGAAACGAAAGAAAGGCCGTAAAAAAGACGTTTTTATCCGC |
| | TCTTGGTTTAGTAAAAAACGTCCGAAGAGAAAATGCCATTCGAAACGAAAGTGCTTT |
| | TGCAAGGAAATCGTCGTCAGAAAGCAAATCGTCCGTGTAAATATACCTCAAAATGTT |
| | TTA |
| SEQ ID NO: 4 | MPALDEWSSIQQIDMEVFVLGRPELICRKKGRKKDVFIRSWFSKKRPKRKCHSKRKC |
| | FCKEIVVRKQIVRVNIPQNVL |
| SEQ ID NO: 5 | ATGAAACACAGAAAACCGTTCAGGTTCAGTGGTGCTTCAAAAAAAGACGAGGACTGC |
| | AAACCACCTAAAATTAGCAGAGAAACGGAAGAACTTCTCAAACTGATTAAGGAATTA |
| | GTCGCCATCATCCCGCTCGTTTTCGCAAACCCGTCTGTGGCTAATGTAACTTCATTG |
| | CAACAGATTTTACAGCGATTATTAGCTCTCGCAAATAAATTGAGACTTAGAGGCTCG |
| | GCTAAGACAGATTTATTAGCGGCGTTGGAACTGGCTATCGTGGCGTCGGAAGCCACT |
| | CTTTTCTCCCCGATCGGTGTTGGAACGACACTGCAACAACTGCTGGAAGTCTTATTG |
| | TCTATTATTTTGCAGGAACCCCTTGATCCTGCTCTTAAAGACAGTTTGATCAGTGCA |
| | ATCAGAAATGCCGAAACGGCTATCAGTATTGCGTTGGGT |
| SEQ ID NO: 6 | MICHRKPFRFSGASKKDEDCKPPKISRETEELLKLIKELVAIIPLVFANPSVANVTS |
| | LQQILQRLLALANKLRLRGSAKTDLLAALELAIVASEATLFSPIGVGTTLQQLLEVL |
| | LSIILQEPLDPALKDSLISAIRNAETAISIALG |
| SEQ ID NO: 7 | ATGGCGGTTATATCAACTGGACCCATAGAAAATAATTATGTCAGTGGTATTCGGCCT |
| | ACTCATCGAGTTACCGTGAAAATTGATAATCGTGATACTGTGAATTCTTCTACGGTA |
| | TTGATTCAGGGTTTTTATCTAAATGGTACAAGAACGTTATATGTGCTTGATTTTATA |
| | ACTGTAAATTCAAATGAAGTGATTACAAAAGATTATTATGCTGATTTTAATTCATTT |
| | GAGTTTGTTTTTACCACTGAAAGTGTTACAGAAAATGAGATTCAAGTTTCAGTCTGG |
| | GGTAAAAATTCAATGGGGCAGTTAGTGACAGCTCACCGTGTTGTATCTTCCGAATTG |
| | CTTGTAGCAAAAGGCGCG |
| SEQ ID NO: 8 | MAVISTGPIENNYVSGIRPTHRVTVKIDNRDTVNSSTVLIQGFYLNGTRTLYVLDFI |
| | TVNSNEVITKDYYADFNSFEFVFTTESVTENEIQVSVWGKNSMGQLVTAHRVVSSEL |
| | LVAKGA |
| SEQ ID NO: 9 | TTGGGAAATTTATTGTTGCGTAAAAGATATCGCTTGACCCAGGTGGCAAGGAAAAAA |
| | AAGAAGGAAAGAGATCAAAAGATGGGAGCGTTCCGTTTTATGCCCATTTATCGTACA |
| | GGAACGAGCTGCATTCGTAACAAAAAGGGAAATAAACGTATTTATAGACAGGGTAGA |
| | AGAAGAGAGAGAATATGCGCTTATAGACATCATTTGCACGCTGAGCGGGTGCCCTCA |
| | GGTTTATCAAATAAAAAAATCTGTTTTATGAAATTCAAAGGTCAACGAAGACTGCGA |
| | GGCGGCGAACAGGAGCCTCAAGGCAATTCAGGAGGAGCAGTTCAA |
| SEQ ID NO: 10 | LGNLLLRICRYRLTQVARKKKKERDQICMGAFRFMPIYRTGTSCIRNKKGNICRIYR |
| | QGRRRERICAYRHHLHAERVPSGLSNKKICFMKFKGQRRLRGGEQEPQGNSGGAVQ |
| SEQ ID NO: 18 | MGNLLLRICRYRLTQVARKKKKERDQICMGAFRFMPIYRTGTSCIRNKKGNICRIYR |
| | QGRRRERICAYRHHLHAERVPSGLSNKKICFMKFKGQRRLRGGEQEPQGNSGGAVQ |
| SEQ ID NO: 11 | GAAACGGGAGTGGTGAAATCATTGATGCTCAGCGCATTGTTGCGGATGAGCAACTAG |
| | ATTCTTGAAACACAACATATGTACAGAGATAGAACCACAATCGTAACAAATGGTTGA |
| | GACATAAAATAGAGGGAACAGGATCTTGAGAAAGATCTCATTGTTCACAAAAAAGCT |
| | TGATTTTACTAGAAAGGAGGGAGTATCCA |
| SEQ ID NO: 12 | CTATATACATGCGCAAAAAACGGCTTCAAACTGCTTCATAATTACGGCACGTTTCTT |
| | CTGGCGCCTTCGGCTGTTCCTTGGTGTGAACCAAGGTAACAGCCGGGGGCGCTATTT |
| | TTATATAACTAGATGAATGTACCTGTACAAAGACCCATTTTTATCCAAAATTAGATC |
| | ATTGCCTATCAACCACAGGACAGATGTCC |
| SEQ ID NO: 13 | AGCGTTACAAGTTGGAAGCCCGGTTTGGAAATACAGAAAATCGATATTAAAGCTTAT |
| | GTACAAGCATCCAATAATAATTCTTGTGTGGTGATTCACCCTTTTCGCTTCAGTAAA |
| | TATATTGTTAATATCTGCGAAACGGGGCGATGATCCACCTGTCACCTCTACAGTAGG |
| | GAGAAATGTGAAGGAGGAGATATTTGAAC |
| SEQ ID NO: 14 | AGCGGTATTTTTTGTGCCCCACAAAAAAGGCTCCCTTATCAAAAGGGGTTTTTATCA |
| | CATAGGAAATGTCCACACGTATATATAGATGTTACATATTATATAAATCGTGAACAT |
| | TCGAATCTCAATACTAGTTATAGAAGAGGTGGCATTAGTGATAGGATTATAGCTTCG |
| | TTACTTTAGACAAAAGGAGAATCCAATAT |
| SEQ ID NO: 15 | ATTTATTTTTTTGAAAAATTACAGGGGATTCAGTCCCACTTTCAGTAAATTCAGAAA |
| | GAAAAATAATGTAACGGCGAAATGGAAGTGAGCATTAAAAATTTATTTTTTTGGAAA |
| | AAAATTTAAGGAGGTCATCTGTCCAATCAGGTTCGTTTAGATTCCATAAGATAATGA |
| | AACTGTACTTAATTATGGAGGTGTCAGTA |
| SEQ ID NO: 33 | ATGGTAGTATTATCTACTGGACCTATTGCAAACGATCCTGTTCTAGGAGTCAGACCC |
| | ACCCAACTGGTCACAGTAAAAATAGATAACCGAGATTCTGTAAATTCTTCTATCGTT |
| | TTGATCGAGGGTTTTATTTTAAACGGTAGCAGAACATTATATGTACAACAATTAGTG |
| | GTAGTGGGACCAAATGCGGTTATAACGAGGAATTTCTTTGCAAATGTAGACGCATTT |
| | GAATTCGTTTTTACCACTAGCGGACCAGCAGAGAATGAAACTCAAATTTCTGTTTGG |
| | GGTAAAGATGCATTG |
| SEQ ID NO: 34 | MVVLSTGPIANDPVLGVRPTQLVTVKIDNRDSVNSSIVLIEGFILNGSRTLYVQQLV |
| | VVGPNAVITRNFFANVDAFEFVFTTSGPAENETQISVWGKDAL |
| SEQ ID NO: 39 | ATGTTTACCACTAGCGGACCAGCAGAGAATGAAACTCAAATTTCTGTTTGGGGTAAA |
| | GATGCATTG |
| SEQ ID NO: 40 | MFTTSGPAENETQISVWGKDAL |
| SEQ ID NO: 41 | ATGACTCAAATTTCTGTTTGGGGTAAAGATGCATTG |
| SEQ ID NO: 42 | MTQISVWGKDAL |
| SEQ ID NO: 45 | ATGCAAATTTCTGTTTGGGGTAAAGAT |
| SEQ ID NO: 49 | MQISVWGK(D/N) |

TABLE 2
Additional Exemplary Paenibacillus N-Terminal Targeting Sequences
Sequence

| Identifier | Sequence |
|---|---|
| SEQ ID NO: 19 | ATGGTAGTATTATCTACTGGACCTATTGCAAACGATCCTGTTCTAGGAGTCAGACCCACCCAA |
| | CTGGTCACAGTAAAAATAGATAACCGAGATTCTGTAAATTCTTCTATCGTTTTGATCGAGGGT |
| | TTTATTTTAAACGGTAGCAGAACATTATATGTACAACAATTAGTGGTAGTGGGACCAAATGCG |
| | GTTATAACGAGGAATTTCTTTGCAAATGTAGACGCATTTGAATTCGTTTTTACCACTAGCGGA |
| | CCAGCAGAGAATGAAACTCAAATTTCTGTTTGGGGTAAAGATGCATTGGGGCAATTAGTACCT |
| | GCCCATCGGTTAGTATCTGACGAACTTTTAGGAACCGATCGAGGAATCCAAGGACCTCAAGGA |
| | GTTCAGGGAGCCCAAGGCGACCAAGGTGACCAAGGACCTCAGGGTGTTCAAGGACCTCAAGGA |
| | GTTCAGGGAGCCCAAGGAGACCAAGGAGTTCAAGGCGTACAAGGAGACCAAGGACCTCAAGGA |
| | GTCCAAGGCGACCAAGGTGACCAAGGACCTCAAGGAGTTCAAGGAGCGCAAGGTGACCAAGGC |
| | CCTCAAGGAGTTCAGGGAGCCCAAGGTGACCAAGGACCTCAAGGCGTTCAGGGAGCGCAAGGT |
| | GACCAAGGACCTCAAGGTGATCAAGGACCTCAGGGAGTTCAAGGAGACCAAGGCGATCAAGGA |
| | CCACAGGGAGTTCAAGGCGTACAAGGTGATCAAGGACCTCAGGGTGTTCAAGGAGACCAAGGC |
| | GACCAAGGACCTCAGGGTGTTCAAGGCGTACAAGGTGACCAAGGACCTCAGGGTGTTCAAGGC |
| | GTACAAGGTGACCAAGGACCTCAGGGAGTTCAAGGAGACCAAGGCGATCAAGGACCACAGGGA |
| | GTTCAAGGCGTACAAGGTGATCAAGGACCTCAGGGTGTTCAAGGAGACCAAGGCGACCAAGGA |
| | CCTCAGGGTGTTCAAGGCGTACAAGGTGACCAAGGACCTCAGGGTGTTCAAGGCGTACAAGGT |
| | GACCAAGGACCTCAGGGTGTTCAAGGCGTACAAGGTGACCAAGGACCTCAAGGAGTTCAGGGA |
| | GCCCAAGGTGACCAAGGACCACAGGGAGTTCAAGGCGACCAAGGACCTCAAGGACCTCAAGGA |
| | GTTCAAGGTGACCAAGGACCTCAGGGCGTTCAAGGATCCCAAGGTGATCAAGGACCTCAAGGA |
| | GTTCAAGGCGTACAAGGACCTCAAGGAGTTCAAGGCGTACAAGGCGACCAAGGACCTCAAGGT |
| | GTTCAGGGAGCCCAAGGCGACCAAGGCCCTCAAGGAGTTCAAGGAGTCCAAGGTGACCAAGGA |
| | CCACAGGGAGTTCAAGGACCGCAAGGTGACCAAGGACCACAGGGAGTTCAGGGAGTCCAAGGC |
| | GACCAAGGACCTCAAGGAGTCCAAGGCGACCAAGGTGACCAAGGACCTCAAGGAGTTCAAGGA |
| | GCGCAAGGTGACCAAGGCCCTCAAGGAGTTCAGGGAGCCCAAGGTGACCAAGGACCTCAAGGC |
| | GTTCAGGGAGCGCAAGGTGACCAAGGACCTCAAGGTGATCAAGGACCTCAGGGAGTTCAAGGA |
| | GACCAAGGCGATCAAGGACCACAGGGAGTTCAAGGCGTACAAGGTGATCAAGGACCTCAGGGT |
| | GTTCAAGGAGACCAAGGCGACCAAGGACCTCAGGGTGTTCAAGGCGTACAAGGTGACCAAGGA |
| | CCTCAGGGTGTTCAAGGCGTACAAGGTGACCAAGGACCTCAGGGTGTTCAAGGCGTACAAGGT |
| | GACCAAGGACCTCAAGGAGTTCAGGGAGCCCAAGGTGACCAAGGACCACAGGGAGTTCAAGGC |
| | GACCAAGGACCTCAAGGACCTCAAGGAGTTCAAGGTGACCAAGGACCTCAGGGCGTTCAAGGA |
| | TCCCAAGGTGATCAAGGACCTCAAGGAGTTCAAGGCGTACAAGGACCTCAAGGAGTTCAAGGC |
| | GTACAAGGCGACCAAGGACCTCAAGGTGTTCAGGGAGCCCAAGGCGACCAAGGCCCTCAAGGA |
| | GTTCAAGGAGTCCAAGGTGACCAAGGACCACAGGGAGTTCAAGGACCGCAAGGTGACCAAGGA |
| | CCACAGGGAGTTCAGGGAGTCCAAGGCGACCAAGGACCTCAAGGTGACCAAGGACCTCAAGGT |
| | GACCAAGGACCTCAAGGTGTTCAAGGTGACCAAGGACCTCAAGGAGTTCAGGGAGCCCAAGGC |
| | GACCAAGGACCTCAAGGAGTTCAAGGACCGCAAGGTGACCAAGGACCTCAAGGAGTTCAAGGC |
| | GTACAAGGTGATCAAGGACCTCAAGGAGTTCAAGGCGTACAAGGTGACCAAGGACCACAGGGT |
| | GTTCAAGGCGTACAAGGTGACCAAGGACCTCAAGGTGTTCAAGGAGTCCAAGGTGATCAAGGA |
| | CCTCAAGGAGTTCAGGGAGCCCAAGGCGACCAAGGACCTCAGGGAGTTCAGGGAGCCCAAGGC |
| | GACCAAGGACCTCAGGGAGTTCAGGGAGCCCAAGGTGACCAAGGACCTCAGGGCGTTCAAGGA |
| | GTACAAGGTGACCAAGGATCTCAAGGAGTTCAAGGACCGCAAGGTGACCAAGGACCTCAAGGA |
| | GTTCAAGGCGTACAAGGTGACCAAGGACCTCAAGGAGTTCAAGGAGTCCAAGGTGACCAAGGA |
| | CCTCAAGGTGTTCAGGGAGCCCAAGGTGGCCAAGGACCTCAGGGCGTTCAAGGCGACCAAGGT |
| | GACCAAGGACCACAGGGTGTTCAAGGATCTCAAGGTGACCAAGGACCACAAGGCGTTCAAGGA |
| | GCCCAAGGCGACCAAGGACCACAGGGTGTTCAAGGCGTACAAGGTGACCAAGGCCCTCAAGGA |
| | GTTCAAGGAGTTCAAGGTGACCAAGGACCACAGGGAGTTCAAGGTGTTCAAGGACCGCAAGGT |
| | GACCAAGGACCACAGGGTGTTCAAGGAGCCCAAGGCGACCAAGGACCACAGGGTGTTCAAGGA |
| | GTGCAAGGTGACCAAGGACCGCAAGGCGACCAAGGTGACCAAGGACCTCAAGGTGTTCAGGGA |
| | GTCCAAGGCGACCAAGGACCTCAAGGTGTTCAGGGAGTCCAAGGCGACCAAGGACCTCAAGGT |
| | GTTCAAGGTGACCAAGGACCACAGGGAGTTCAGGGAGCCCAAGGTGACCAAGGACCTCAGGGA |
| | GTTCAAGGTGACCAAGGTGACCAAGGACCTCAAGGAGTTCAAGGTGTACAAGGTGACCAAGGA |
| | CCTCAAGGTGTTCAGGGAGCCCAAGGTGACCAAGGACCTCAGGGCGTACAAGGCGACCAAGGT |
| | GACCAAGGACCACAGGGTGTTCAAGGCGTACAAGGTGATCAAGGACCTCAAGGAGTTCAAGGC |
| | GTACAAGGTGACCAAGGACCACAGGGTGTTCAAGGCGTACAAGGTGACCAAGGACCACAGGGT |
| | GTTCAAGGCGACCAAGGTGACCAAGGACCTCAAGGCGTACAAGGCGATCAAGGACCTCAGGGT |
| | GTTCAAGGACCTCAGGGTGTTCAAGGACCTCAGGGTGTTCAAGGACCTCAAGGCGACCAAGGA |
| | GCTCAAGGTGTTCAAGGACCACAAGGTGACCAAGGACCGCAAGGCATTCTGTAA |
| SEQ ID NO: 20 | MVVLSTGPIANDPVLGVRPTQLVTVKIDNRDSVNSSIVLIEGFILNGSRTLYVQQLVVVGPNA |
| | VITRNFFANVDAFEFVFTTSGPAENETQISVWGKDALGQLVPAHRLVSDELLGTDRGIQGPQG |
| | VQGAQGDQGDQGPQGVQGPQGVQGAQGDQGVQGVQGDQGPQGVQGDQGDQGPQGVQGAQGDQG |
| | PQGVQGAQGDQGPQGVQGAQGDQGPQGDQGPQGVQGDQGDQGPQGVQGVQGDQGPQGVQGDQG |
| | DQGPQGVQGVQGDQGPQGVQGVQGDQGPQGVQGDQGDQGPQGVQGVQGDQGPQGVQGDQGDQG |
| | PQGVQGVQGDQGPQGVQGVQGDQGPQGVQGVQGDQGPQGVQGAQGDQGPQGVQGDQGPQGPQG |
| | VQGDQGPQGVQGSQGDQGPQGVQGVQGPQGVQGVQGDQGPQGVQGAQGDQGPQGVQGVQGDQG |
| | PQGVQGPQGDQGPQGVQGVQGDQGPQGVQGDQGDQGPQGVQGAQGDQGPQGVQGAQGDQGPQG |
| | VQGAQGDQGPQGDQGPQGVQGDQGDQGPQGVQGVQGDQGPQGVQGDQGDQGPQGVQGVQGDQG |
| | PQGVQGVQGDQGPQGVQGVQGDQGPQGVQGAQGDQGPQGVQGDQGPQGPQGVQGDQGPQGVQG |
| | SQGDQGPQGVQGVQGPQGVQGVQGDQGPQGVQGAQGDQGPQGVQGVQGDQGPQGVQGPQGDQG |
| | PQGVQGVQGDQGPQGDQGPQGDQGPQGVQGDQGPQGVQGAQGDQGPQGVQGPQGDQGPQGVQG |
| | VQGDQGPQGVQGVQGDQGPQGVQGVQGDQGPQGVQGVQGDQGPQGVQGAQGDQGPQGVQGAQG |
| | DQGPQGVQGAQGDQGPQGVQGVQGDQGSQGVQGPQGDQGPQGVQGVQGDQGPQGVQGVQGDQG |
| | PQGVQGAQGGQGPQGVQGDQGDQGPQGVQGSQGDQGPQGVQGAQGDQGPQGVQGVQGDQGPQG |
| | VQGVQGDQGPQGVQGVQGPQGDQGPQGVQGAQGDQGPQGVQGVQGDQGPQGDQGDQGPQGVQG |
| | VQGDQGPQGVQGVQGDQGPQGVQGDQGPQGVQGAQGDQGPQGVQGDQGDQGPQGVQGVQGDQG |
| | PQGVQGAQGDQGPQGVQGDQGDQGPQGVQGVQGDQGPQGVQGVQGDQGPQGVQGVQGDQGPQG |
| | VQGDQGDQGPQGVQGDQGPQGVQGPQGVQGPQGVQGPQGDQGAQGVQGPQGDQGPQGIL |
| SEQ ID NO: 21 | MVVLSTGPIANDPVLGVRPTQLVTVKIDNRDSVNSSIVLIEGFILNGSRTLYVQQLVVVGPNA |
| | VITRNFFANVDAFEFVFTTSGPAENETQISVWGKDALGQLVPAHRLVSDELLGTDRGIQGPQG |
| | VQGAQGDQGDQGPQGVQGPQGVQGAQGDQGVQGVQGDQGPQGVQGDQGDQGPQGVQGAQGDQG |
| | PQGVQGAQGDQGPQGVQGAQGDQGPQGDQGPQGVQGDQGDQGPQGVQGVQGDQGPQGVQGDQG |
| | DQGPQGVQGVQGDQGPQGVQGVQGDQGPQGVQGVQGDQGPQGVQGAQGDQGPQGVQGDQGPQG |
| | LQGVQGDQGPQGVQGSQGDQGPQGVQGVQGPQGGQGVQGDQGPQGVQGAQGDQGPQGVQGDQG |
| | DQGPQGVQGAQGDQGPQGVQGDQGDQGPQGVQGDQGTKELKEYKVTICELKEFICEPKVTKDL |
| | RAFKATKVTKDHRVFICEFKVTKDLKEFKEYKVTKDHRVFKAYKVTICDLKVFKATKVTKDLK |
| | AYKAIKDLRVFKDLRVFKDLRVFICDLKATKELKVFKDHKVTICDRKAFCKLKVKVYLDDSKV |
| | IITFGSFFVLS |
| SEQ ID NO: 22 | MVVLSTGPIANDPVLGVRPTQLVTVKIDNRDSVNSSIVLIEGFILNGSRTLYVQQLVVVGPNA |
| | VITRNFFANVDAFEFVFTTSGPAENETQISVWGKDALGQLVPAHRLVSDELLGTDRGIQGPQG |
| | VQGAQGDQGDQGPQGVQGPQGVQGAQGDQGVQGVQGDQGPQGVQGDQGDQGPQGVQGAQGDQG |
| | PQGVQGAQGDQGPQGVQGAQGDQGPQGDQGPQGVQGDQGDQGPQGVQGVQGDQGPQGVQGDQG |
| | DQGPQGVQGVQGDQGPQGVQGVQGDQGPQGVQGVQGDQGPQGVQGAQGDQGPQGVQGDQGPQG |
| | PQGVQGDQGPQGVQGAQGDQGPQGVQGVQGPQGVQGVQGDQGPQGVQGAQGDQGPQGVQGVQG |
| | DQGPQGVQGAQGDQGPQGVQGVQGDQGPQGVQGDQGDQGPQGVQGAQGDQGPQGVQGAQGDQG |
| | PQGVQGAQG |
| SEQ ID NO: 23 | ATGCCTGCCTTGGATGAATGGAGTAGTATACAACAAATCGATATGGAGGTGTTTGTATTGGGT |
| | CGTCCCGAATTGAAACGAAAGAAAGGCCGTAAAAAAGACGTTTTTATCCGCTCTTGGTTTAGT |
| | AAAAAACGTCCGAAGAGAAAATGCCATTCGAAACGAAAGTGCTTTTGCAAGGAAATCGTCGTC |
| | AGAAAGCAAATCGTCCGTGTAAATATACCTCAAAATGTTTTAGGTATAACAGGCGCAACTGGA |
| | GCTATAGGTGTAGCAGGTAACGTAGGTGCAGCGGGCACTGTGGGTGCTGCTGGAGCCGTCGGA |
| | ACTGCGGGAAATGTCGGGGCTGCCGGTAATGTGGGTACTGCGGGCACCGTTGGGACTGCCGGA |
| | AATGTAGGCGCAGCGGGGGCTGTGGGCACTGCGGGCGCTGTTGGAGCTGCGGGTGCGGTAGGA |
| | CCAGTAGGTCCCGTAGGTCCTGCGGGCATTCCAGGGGCAGTCGGTCCAGCAGGTCCTGCGGGC |
| | GTTGCAGGGGCGGTCGGTCCTGTAGGTCCTGCGGGTGCGGTAGGTGCCACTGGGGCTACGGGT |
| | ACCGCAGGAGCGACGGGGTCCACCGGGGCTACGGGAGCTACAGGAACCGCAGGTGGAATAGCT |
| | CAGTTTGGTTATATCTACAACTTAGGATCCCGAGTCGTTCCAATAGAAGCGGATGTCATTTTC |
| | GATACGAACGGTATACTTACACCTGGAATTACCCACGCTCCCGGCACTACGCAGATTGCAGTT |
| | ACCGATGCGGGGAACTATGAAGTTAACTTTTCAGTATCGGGTGTAGAGCCAGGCCAATTTGCC |
| | ATATTTATCAATGGCACTCTGGCAGCAGGAACCATATACGGCTCAGGAGCTGGTACGCAGCAA |
| | AACACAGGGCAGGCCATCCTCGCTCTAGCATCCGGTGATGTTCTTACCCTGCGAAATCATAGC |
| | TCTGCCGCTGCGGTTACCCTGCAAACCTTGGCTGGAGGTACCCAAGCCAACGTAAACGCTTCT |
| | GTCGTTATCAAAAAATTAAGTTAG |
| SEQ ID NO: 24 | MPALDEWSSIQQIDMEVFVLGRPELKRKKGRKKDVFIRSWFSKICRPICRKCHSKRKCFCKEI |
| | VVRKQIVRVNIPQNVLGITGATGAIGVAGNVGAAGTVGAAGAVGTAGNVGAAGNVGTAGTVGT |
| | AGNVGAAGAVGTAGAVGAAGAVGPVGPVGPAGIPGAVGPAGPAGVAGAVGPVGPAGAVGATGA |
| | TGTAGATGSTGATGATGTAGGIAQFGYIYNLGSRVVPIEADVIFDTNGILTPGITHAPGTTQI |
| | AVTDAGNYEVNFSVSGVEPGQFAIFINGTLAAGTIYGSGAGTQQNTGQAILALASGDVLTLRN |
| | HSSAAAVTLQTLAGGTQANVNASVVIKKLS |
| SEQ ID NO: 25 | ATGAAACACAGAAAACCGTTCAGGTTCAGTGGTGCTTCAAAAAAAGACGAGGACTGCAAACCA |
| | CCTAAAATTAGCAGAGAAACGGAAGAACTTCTCAAACTGATTAAGGAATTAGTCGCCATCATC |
| | CCGCTCGTTTTCGCAAACCCGTCTGTGGCTAATGTAACTTCATTGCAACAGATTTTACAGCGA |
| | TTATTAGCTCTCGCAAATAAATTGAGACTTAGAGGCTCGGCTAAGACAGATTTATTAGCGGCG |
| | TTGGAACTGGCTATCGTGGCGTCGGAAGCCACTCTTTTCTCCCCGATCGGTGTTGGAACGACA |
| | CTGCAACAACTGCTGGAAGTCTTATTGTCTATTATTTTGCAGGAACCCCTTGATCCTGCTCTT |
| | AAAGACAGTTTGATCAGTGCAATCAGAAATGCCGAAACGGCTATCAGTATTGCGTTGGGTGGC |
| | ACGGCAGGAACCCCCGGTCCACAAGGGCCCGCTGGGCCTGCTGGTCCGGGCGGTGCTCCAGGA |
| | CCTGTCGGTGGACCAGGGCCGGTGGGTGCGGCAGGACCAGCAGGTCCAGTTGGACCTGCTGGT |
| | CCTGTCGGACCTGTCGGGGCTGCCGGACCTGTTGGAGCCGCCGGACCTGTTGGAGCCGCCGGA |
| | CCTATCGGCGCCGCTGGGCCAGTAGGCGCCGCCGGGGCTGCTGGAGCCACCGGGGCTACAGGA |
| | GCTACAGGCGCGGCAGGACCTGCCGGGGGGGCTACCGGGGCCACGGGCGCCGTTGGAGCCACA |
| | GGCGCTACGGGCGCAGCGGGGGTCGCTGGGGCTACAGGAACTACGGGCACGGCGGGCGCTGTC |
| | GGAGCTACCGGGGCCACGGGCACGGCGGGGGCCATTGGAGCTACCGGGGCCACAGGCACGGCG |
| | GGGGCCGTCGGAGCTACCGGGGCCACAGGCACGGCGGGCGCTGTCGGAGCTACCGGGGCCACG |
| | GGTACAGCAGGGGTTACTGGAGCCACCGGTTCGGGGGCAATCATTCCATTTGCTTCGGGTGGA |
| | CCAGCAATTTTGACAACCATTGTCGGCGGGCTGGTTGGAACCACAAGTTTGATCGGCTTTGGA |
| | AGCTCAGCAACAGGCATTAGCCTTGTGGGTGGAACCATTGACCTGACAGGCACACTTGCAGGG |
| | CCACTGATTAACTTTGCTTTTTCTGTACCACGGGATGGCGTAATTACATCCATCGCTGGATAT |
| | TTTAGTACAACAGCTGCGCTAACTCTCGTTGGATCAACCGCGACGATTACTGCCCAGTTGTTT |
| | AGTTCGACTACACCTGATAACACCTTTACAGCGGTCCCTGGGGCTACCGTTACATTAGCTCCA |
| | CCACTGACTGGCATCATTGCCTTGGGTACCATTTCCAATGGCATCACTACCGGATTGGCTATA |
| | CCAGTAACCGCGCAGACTCGTCTGCTCCTTGTCTTCTCTGCAACAGCTACGGGACTCTCCCTC |
| | GTAAACACCATCGTGGGTTATGCGAGCGCAGGCATTACCATCACCTGA |
| SEQ ID NO: 26 | MKHRKPFRFSGASKKDEDCKPPKISRETEELLKLIKELVAIIPLVFANPSVANVTSLQQILQR |
| | LLALANKLRLRGSAKTDLLAALELAIVASEATLFSPIGVGTTLQQLLEVLLSIILQEPLDPAL |
| | KDSLISAIRNAETAISIALGGTAGTPGPQGPAGPAGPGGAPGPVGGPGPVGAAGPAGPVGPAG |
| | PVGPVGAAGPVGAAGPVGAAGPIGAAGPVGAAGAAGATGATGATGAAGPAGGATGATGAVGAT |
| | GATGAAGVAGATGTTGTAGAVGATGATGTAGAIGATGATGTAGAVGATGATGTAGAVGATGAT |
| | GTAGVTGATGSGAIIPFASGGPAILTTIVGGLVGTTSLIGFGSSATGISLVGGTIDLTGTLAG |
| | PLINFAFSVPRDGVITSIAGYFSTTAALTLVGSTATITAQLFSSTTPDNTFTAVPGATVTLAP |
| | PLTGIIALGTISNGITTGLAIPVTAQTRLLLVFSATATGLSLVNTIVGYASAGITIT |
| SEQ ID NO: 27 | ATGGCGGTTATATCAACTGGACCCATAGAAAATAATTATGTCAGTGGTATTCGGCCTACTCAT |
| | CGAGTTACCGTGAAAATTGATAATCGTGATACTGTGAATTCTTCTACGGTATTGATTCAGGGT |
| | TTTTATCTAAATGGTACAAGAACGTTATATGTGCTTGATTTTATAACTGTAAATTCAAATGAA |
| | GTGATTACAAAAGATTATTATGCTGATTTTAATTCATTTGAGTTTGTTTTTACCACTGAAAGT |
| | GTTACAGAAAATGAGATTCAAGTTTCAGTCTGGGGTAAAAATTCAATGGGGCAGTTAGTGACA |
| | GCTCACCGTGTTGTATCTTCCGAATTGCTTGTAGCAAAAGGCGCGGGACCGACAGGGCTAACG |
| | GGAGCCACTGGCGCTACCGGAGCTACTGGCGTCACGGGTGTTACCGGAGTCACTGGCGCTACC |
| | GGAACTACGGGCGTTATGGGTGATACCGGAGTCACTGGAGTTACCGGAGTTACTGGCGTTACC |
| | GGGGCTATCGGAGTCACTGGCGCTATCGGAGTCACGGGGGCTACCGGAGCCACAGGAGTTACG |
| | GGGGCCACTGGAGTTACCGGGGCTATTGGAGTTACTGGCGCTATCGGAGTCACTGGCGCTACC |
| | GGAGCTACTGGCGTTACTGGGGCTACTGGCGCTACTGGAGTCACAGGAGTTACCGGGGCTACT |
| | GGCGTTACCGGAGTTACCGGAGTTACTGGCATCACCGGGGCTATCGGAGCTACTGGCGTTACC |
| | GGAGCTACTGGCGTCACGGGTATTACCGGAGTCACTGGCGTTACCGGGGCTACTGGCGTTACT |
| | GGAGTTACTGGCATCACAGGCGTTACCGGAGTTACTGGTGTTACTGGTGTTACTGGAGCTACT |
| | GGCGTTACCGGGGCTACTGGCGCTACCGGAGCCACTGGCGTTACTGGAGTTACTGGCGTTACT |
| | GGCGCTACTGGAGCTACTGGTGTTACCGGGGCTACCGGGGCTACCGGTGTCACGGGTGATACC |
| | GGTGTCACTGGCGCTACCGGGGCTACCGGAGTTTCTGGCGCTACTGGGGCTACTGGTGTCACG |
| | GGTGATACCGGAGTTACCGGAGCTACTGGCGCTACAGGTGCTACCGGAGTTACTGGCGGAACA |
| | GGTGCAACCGGAGTTACTGGAGTTACTGGCGTTACCGGGGCTATCGGAGTCACTGGCGCTACT |
| | GGAGCTACTGGAGCTGCTGGAATCACGGGTGTTACCGGAGTTACTGGCATCACCGGTGCTACC |
| | GGGGCTACGGGCGCTACCGGAGTTACTGGCATCACAGGAGTCACTGGCGCTACCGGAGTTACT |
| | GGCGTAACAGGTGCAACCGGAGTTACTGGAGTTACCGGGGCTATCGGAGTTACTGGTGTCACC |
| | GGAGCTACTGGCGTCACGGGTGTTACCGGAGTCACTGGCGCTACCGGAGCTACTGGCGTTACG |
| | GGTGTTACCGGAGTTACCGGAGTTACTGGCGTTACCGGAGCTACTGGCGTTACCGGAGTTACT |
| | GGAGTTACTGGAGTTATTGGAGTTACTGGAGTTACTGGAGTTACTGGAGTTACTGGAGTTACC |
| | GGAGTTACCGGAGTTACTGGAGTTACCGGGGCTATCGGAGTCACTGGCGCTATCGGAGTCACG |
| | GGGGCTACCGGGGTCACTGGCGCTACCGGAGCTACTGGCGTAACAGGGGCTACTGGAGTTACC |
| | GGGGCTATCGGAGTCACTGGCGCTACTGGAGCTGCTGGAATCACGGGTGTTACCGGAGTCACT |
| | GGTGTTACTGGAGTTACCGGAGCTACTGGCATCACGGGTGATACCGGAGTCACTGGCGCTACC |
| | GGAGCTACTGGCGTTACGGGTGTTACCGGAGTCACTGGGGCTACCGGAGCTACTGGCGTCACG |
| | GGTGATACCGGAGTTACTGGAGTCACTGGCGCTACCGGAGTTACTGGCGTAACAGGTGCAGCC |
| | GGAGTTACTGGCATCACGGGGGCTACCGGAGTTACTGGAGTTACCGGGGCTATTGGAGTCACT |
| | GGCGCTATCGGAGTCACGGGGGCTACCGGAGCCACAGGAGTTACGGGTATTACCGGAGCTACT |
| | GGCGCTACTGGAGCCACAGGTGCTACCGGAGTTACTGGAGTTACTGGCGCTACCGGAGCTACT |
| | GGCGCTACTGGCGTCACGGGTTCTACTGGGGTCACTGGCGCTACTGGCGTTACCGGAGCTACT |
| | GGCGTCACGGGTTCTACTGGGGTCACTGGCGCTACTGGCGTTACCGGAGCTACTGGCGTCACG |
| | GGTATTACCGGAGTCACTGGCGTTACCGGAGTTACTGGTGCTACTGGAGCTACTGGCGTTACC |
| | GGGGCTACCGGAGTCACTGGGGCTACCGGAGCTACTGGCGTCACGGGTATTACCGGAGTCACT |
| | GGGGCTACCGGAGCTACTGGCGTCACGGGTGTTACCGGAGTCACCGGAGTCACTGGAGTTACT |
| | GGAGTTACTGGCGCTACCGGAGCTACTGGCGTTACCGGAGCTACTGGCGCTACTGGCGTCACG |
| | GGTGATACCGGAGTCACTGGGGCTACCGGAGTTACCGGAGTCACTGGCGCTACTGGGGCTACT |
| | GGTGTCACGGGTGTTACCGGAGTCACTGGCGCTACCGGGGCTACTGGTGTCACGGGTGTTACC |
| | GGGGCTACCGGAGCTACTGGCGACACGGGTGTTACCGGAGTCACTGGAGTCACTGGAGTTACC |
| | GGAGTTTCTGGCGCTACCGGAGTTACCGGAGTTTCTGGCGCTACCGGAGTTACCGGAGCTACT |
| | GGCGTTACCGGGGCTGGGGCTACCGGAGCTACTGGCGCTACTGGAGTCACAGGTGTTACCGGA |
| | GTCACTGGCGCTACCGGAGCTACTGGCGCTACTGGAGTCACGGGTGTTACCGGAGTCACTGGC |
| | GCTACCGGGGCTACTGGTGTCACGGGTGTTACCGGGGCTACCGGAGCTACTGGCGACACGGGT |
| | GTTACCGGAGTCACTGGAGTCACTGGAGTTACCGGAGTTTCTGGCGCTACCGGAGTTACCGGA |
| | GCTACTGGCGTTACCGGGGCTGGGGCTACCGGAGCTACTGGCGCTACTGGAGTCACAGGTGTT |
| | ACCGGAGTCACTGGCGCTACCGGAGCTACTGGCGCTACTGGAGTCACGGGTGTTACCGGAGTC |
| | ACTGGCGCTACCGGGGCTACTGGCGCTACTGGAGTCACGGGTGTTACTGGCGTTACGGGTGTT |
| | ACCGGAGTTTCTGGCATCACCGGTGCTACCGGGGCTATTGGACCTACTGGTGCCACAGGTGTT |
| | GGTATAACAGGTTCAACAGGTTCAACCGGCCCCACTGGCCCACCTCCTACGTTTATAGACGCA |
| | TACTTTAACGGTAATATTCAACCTCAGACAATTGCTTCGGGATCAAACATTTTAAATATTACT |
| | CCAAACCAATCTACTGCACTTACTTATAACGCAGTAACAAGTGTTTTCACAATACAAAATGCG |
| | GGGTTGTATAACATTAGTGTTGTAATAAATCTTGCAACTGCCACACTACCAGAAGCAACAATT |
| | GGGTTATCACTAAATAATTCTACAGCATATCTCGCTCCTGCTGTAACCACGGCAACAAGTGGT |
| | CAATTGGTTTTAGTTCAAATTGAGGCTCTTGCTGTCGGAGATACAATTCAATTTAGAAATATA |
| | TCTGGGTTTCCTATTACCATTGCTAATTCACCAGTAATAGCTAACAGCTCAGGTCATGTAGCT |
| | ATTTCGAGATTCTCAGCTTTTTCATAA |
| SEQ ID NO: 28 | MAVISTGPIENNYVSGIRPTHRVTVKIDNRDTVNSSTVLIQGFYLNGTRTLYVLDFITVNSNE |
| | VITKDYYADFNSFEFVFTTESVTENEIQVSVWGKNSMGQLVTAHRVVSSELLVAKGAGPTGLT |
| | GATGATGATGVTGVTGVTGATGTTGVMGDTGVTGVTGVTGVTGAIGVTGAIGVTGATGATGVT |
| | GATGVTGAIGVTGAIGVTGATGATGVTGATGATGVTGVTGATGVTGVTGVTGITGAIGATGVT |
| | GATGVTGITGVTGVTGATGVTGVTGITGVTGVTGVTGVTGATGVTGATGATGATGVTGVTGVT |
| | GATGATGVTGATGATGVTGDTGVTGATGATGVSGATGATGVTGDTGVTGATGATGATGVTGGT |
| | GATGVTGVTGVTGAIGVTGATGATGAAGITGVTGVTGITGATGATGATGVTGITGVTGATGVT |
| | GVTGATGVTGVTGAIGVTGVTGATGVTGVTGVTGATGATGVTGVTGVTGVTGVTGATGVTGVT |
| | GVTGVIGVTGVTGVTGVTGVTGVTGVTGVTGAIGVTGAIGVTGATGVTGATGATGVTGATGVT |
| | GAIGVTGATGAAGITGVTGVTGVTGVTGATGITGDTGVTGATGATGVTGVTGVTGATGATGVT |
| | GDTGVTGVTGATGVTGVTGAAGVTGITGATGVTGVTGAIGVTGAIGVTGATGATGVTGITGAT |
| | GATGATGATGVTGVTGATGATGATGVTGSTGVTGATGVTGATGVTGSTGVTGATGVTGATGVT |
| | GITGVTGVTGVTGATGATGVTGATGVTGATGATGVTGITGVTGATGATGVTGVTGVTGVTGVT |
| | GVTGATGATGVTGATGATGVTGDTGVTGATGVTGVTGATGATGVTGVTGVTGATGATGVTGVT |
| | GATGATGDTGVTGVTGVTGVTGVSGATGVTGVSGATGVTGATGVTGAGATGATGATGVTGVTG |
| | VTGATGATGATGVTGVTGVTGATGATGVTGVTGATGATGDTGVTGVTGVTGVTGVSGATGVTG |
| | ATGVTGAGATGATGATGVTGVTGVTGATGATGATGVTGVTGVTGATGATGATGVTGVTGVTGV |
| | TGVSGITGATGAIGPTGATGVGITGSTGSTGPTGPPPTFIDAYFNGNIQPQTIASGSNILNIT |
| | PNQSTALTYNAVTSVFTIQNAGLYNISVVINLATATLPEATIGLSLNNSTAYLAPAVTTATSG |
| | QLVLVQIEALAVGDTIQFRNISGFPITIANSPVIANSSGHVAISRFSAFS |
| SEQ ID NO: 29 | TTGGGAAATTTATTGTTGCGTAAAAGATATCGCTTGACCCAGGTGGCAAGGAAAAAAAAGAAG |
| | GAAAGAGATCAAAAGATGGGAGCGTTCCGTTTTATGCCCATTTATCGTACAGGAACGAGCTGC |
| | ATTCGTAACAAAAAGGGAAATAAACGTATTTATAGACAGGGTAGAAGAAGAGAGAGAATATGC |
| | GCTTATAGACATCATTTGCACGCTGAGCGGGTGCCCTCAGGTTTATCAAATAAAAAAATCTGT |
| | TTTATGAAATTCAAAGGTCAACGAAGACTGCGAGGCGGCGAACAGGAGCCTCAAGGCAATTCA |
| | GGAGGAGCAGTTCAAGGGGTGCATGGATTAAGGGGGACCGATGGTAATGCTGGGCATCAAGGC |
| | ATACAAGGTCCGGCTGGGCCACAGGGCATTCCGGGTAGTGCCGGACCCCAGGGCCAGGCGGGC |
| | GCCATAGGCCCCCAAGGTGAACAGGGTCTTCAGGGGGTTCCAGGGATTCAAGGCTTGCAAGGA |
| | GAGGCTGGGCCACAGGGAGAGCAGGGACCACCGCTTAATTTGGATGGGATCACGGTTGTGCCT |
| | GAGGTACAGCGATATTTCTATTTTGCCGATTCAGATCTGACGGGTACGGTTGAAATCCCTATT |
| | TCCCAGTTTACGAATGATGATGGACAGTTGGCAAGTCAGCTTCCAGAATTGGGTGCGAACAGC |
| | TACACGGATTTGTATATTAATGGGGTACTGCAGGAAAGCAGGTTGTACCAGATAAGTAGTACC |
| | ACATTGACTGTTGAATTGGAAGAAGCTCTTGTAATTGCGGGTACGCCGTTTATTTTCGAGGTT |
| | TTTCAATTTACATTAAGAATGGCGAACTGA |
| SEQ ID NO: 30 | MGNLLLRKRYRLTQVARKKKKERDQICMGAFRFMPIYRTGTSCIRNKKGNICRIYRQGRRRER |
| | ICAYRHHLHAERVPSGLSNKKICFMKFKGQRRLRGGEQEPQGNSGGAVQGVHGLRGTDGNAGH |
| | QGIQGPAGPQGIPGSAGPQGQAGAIGPQGEQGLQGVPGIQGLQGEAGPQGEQGPPLNLDGITV |
| | VPEVQRYFYFADSDLTGTVEIPISQFTNDDGQLASQLPELGANSYTDLYINGVLQESRLYQIS |
| | STTLTVELEEALVIAGTPFIFEVFQFTLRMAN |

In addition to the exemplary N-terminal targeting sequences listed on Table 1 (i.e., SEQ ID NOs: 1-10, 18, 33, 34, 39-42, 45 and 49) and in Table 2 (i.e., SEQ ID NOs: 19-30), in further embodiments, variant sequences sharing at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with any of the aforementioned sequences may be used, so long as the sequence retains the capability to target the fusion protein to the spore surface of a Paenibacillus endospore. In some embodiments, a fragment of at least 5, 6, 7, 8, 9, 10, 11, 12, 13, 14, 15, 16, 17, 18, 19, 20, 21, 22, 23, 24, or 25 contiguous amino acids selected from any of the polypeptide sequences listed in Table 1 may be used. In some aspects, the only required functionality is that the sequence maintains the capability to target a fusion protein to the spore surface of a Paenibacillus endospore.

In some embodiments this N-terminal signal sequence, or a variant or fragment thereof, may be used to target a fusion protein to the spore surface of a Paenibacillus endospore which comprises a peptide or polypeptide sequence of interest that is heterologous to the linked N-terminal signal sequence. In some embodiments, the N-terminal signal sequence comprises an amino acid sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with the amino acid sequence of any of the individual sequences listed on Table 1. In some embodiments, the N-terminal signal sequence comprises at least one contiguous sequence of at least 5, 6, 7, 8, 9, 10, 11, 12, 13, 14, 15, 16, 17, 18, 19, 20, 21, 22, 23, 24, or 25 amino acids that is identical to a contiguous sequence of the same number amino acids of any of the individual polypeptide sequences listed on Table 1.

As discussed herein, fusion protein constructs according to several aspects of the disclosure comprise an N-terminal signal sequence or a variant or fragment thereof that targets the fusion protein to the spore surface of a Paenibacillus endospore and a polypeptide sequence that is heterologous to the N-terminal signal sequence. However, in further aspects, any of the disclosed sequences, as well as the sequential variants and fragments thereof according to any of the disclosed aspects, may be used for other purposes. The disclosure's focus on aspects wherein these sequences function as N-terminal spore surface targeting sequences is not to be construed as a disclaimer of other functionalities.

In some embodiments, the N-terminal signal sequence comprises a polypeptide with an amino acid sequence as represented by SEQ ID NO: 2, 4, 6, 8 or 10. In alternative embodiments, the N-terminal signal sequence comprises a fragment of SEQ ID NO: 2, 4, 6, 8 or 10 (e.g., a polypeptide with an amino acid sequence comprising at least one contiguous subsequence found in either SEQ ID NO: 2, 4, 6, 8 or 10). In alternative embodiments, the N-terminal signal sequence comprises a variant of SEQ ID NO: 2, 4, 6, 8 or 10 (e.g., a polypeptide with an amino acid sequence that shares a minimum or exact degree of percentage identity with the sequence represented by SEQ ID NO: 2, 4, 6, 8 or 10). In select embodiments, the N-terminal signal sequence may qualify as both a fragment and as a variant, as defined above (e.g., an N-terminal signal sequence comprising a contiguous subsequence found in SEQ ID NO: 2, 4, 6, 8 or 10 followed by a divergent sequence that falls within a disclosed sequence identity range).

In select embodiments, the N-terminal signal sequence comprises an amino acid sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with the amino acid sequence represented by SEQ ID NO: 2, 4, 6, 8 or 10.

In select embodiments, the N-terminal signal sequence comprises a contiguous sequence of at least 5, 10, 15, 20 or 25 amino acids that is identical to a contiguous sequence of at least 5, 6, 7, 8, 9, 10, 11, 12, 13, 14, 15, 16, 17, 18, 19, 20, 21, 22, 23, 24, or 25 amino acids of the amino acid sequence represented by SEQ ID NO: 2, 4, 6, 8 or 10.

In some embodiments, the N-terminal signal sequence comprises a polypeptide with an amino acid sequence encoded by the nucleotide sequence represented by SEQ ID NO: 1, 3, 5, 7, 9, 33, 39, 41, or 45. In alternative embodiments, the N-terminal signal sequence comprises a fragment of a polypeptide with an amino acid sequence encoded by the nucleotide sequence represented by SEQ ID NO: 1, 3, 5, 7, 9, 33, 39, 41, or 45 (e.g., a polypeptide with an amino acid sequence comprising at least one contiguous subsequence found in a polypeptide with an amino acid sequence encoded by the nucleotide sequence represented by SEQ ID NO: 1, 3, 5, 7, 9, 33, 39, 41, or 45). In alternative embodiments, the N-terminal signal sequence comprises a variant of a polypeptide with an amino acid sequence encoded by the nucleotide sequence represented by SEQ ID NO: 1, 3, 5, 7, 9, 33, 39, 41, or 45 (e.g., a polypeptide with an amino acid sequence that shares a minimum or exact degree of percentage identity with a polypeptide with an amino acid sequence encoded by the nucleotide sequence represented by any of the sequences represented by SEQ ID NO: 1, 3, 5, 7, 9, 33, 39, 41, or 45). In select embodiments, the N-terminal signal sequence may qualify as both a fragment and as a variant, as defined above (e.g., an N-terminal signal sequence comprising a contiguous subsequence found in a polypeptide with an amino acid sequence encoded by the nucleotide sequence represented by SEQ ID NO: 1, 3, 5, 7, 9, 33, 39, 41, or 45 followed by a divergent sequence that falls within a minimum sequence identity range).

In select embodiments, the N-terminal signal sequence comprises a nucleotide sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with the nucleotide sequence represented by SEQ ID NO: 1, 3, 5, 7, 9, 33, 39, 41, or 45.

In select embodiments, the N-terminal signal sequence comprises a nucleotide sequence that hybridizes to a nucleic acid probe complementary to SEQ ID NO: 1, 3, 5, 7, 9 under moderate or high stringency.

In select embodiments, the N-terminal signal sequence comprises a contiguous sequence of at least 5, 6, 7, 8, 9, 10, 11, 12, 13, 14, 15, 16, 17, 18, 19, 20, 21, 22, 23, 24, 25, 26, 27, 28, 29, or 30 nucleotides that is identical to a contiguous sequence of the same number of nucleotides in the polynucleotide sequence represented by SEQ ID NOs: 1, 3, 5, 7, 9, 33, 39, 41, or 45.

With respect to any of the alternative N-terminal targeting sequences contemplated by this disclosure, such as the aforementioned embodiments, the minimum required functionality of such sequences in selected aspects is the capability to target a fusion protein to the spore surface of a Paenibacillus endospore.

### Sporulation-Associated Regulatory Sequences

The disclosure provides multiple upstream regulatory sequences which may be used to express fusion proteins and other constructs according to the disclosure during sporulation (e.g., SEQ ID NOs: 11-15). As described in detail herein, these upstream regulatory sequences may be used to express fusion proteins having an N-terminal targeting sequence which directs a protein of interest to the spore surface of a Paenibacillus endospore. In some aspects, these upstream regulatory sequences (or fragments or variants thereof) may also be used to express any heterologous protein of interest during sporulation regardless of whether the protein of interest includes an N-terminal targeting sequence.

In some aspects, transcription of the protein of interest is controlled by a promoter present in any of the upstream regulatory sequences described herein (e.g., any of SEQ ID NOs: 11-15, or a fragment or variant thereof which remains transcriptionally active during sporulation). In some aspects, a DNA construct may comprise a sequence encoding a protein of interest downstream of any of the regulatory sequences described herein (e.g., any of SEQ ID NOs: 11-15), or a fragment or variant thereof which remains transcriptionally active during sporulation. Such fragments may comprise any contiguous 25, 50, 100, 150 or 200 nucleotides of SEQ ID NOs: 11-15, which remains transcriptionally active during sporulation. Similarly, variants may comprise a sequence having at least 50%, 60%, 70%, 80%, or 90% sequence identity compared to any of SEQ ID NOs: 11-15, 33, 39, 41, or 45 (or a fragment thereof), which remain transcriptionally active during sporulation. DNA encoding the protein of interest and any upstream regulatory sequence(s) may be integrated into the chromosomal DNA of a Paenibacillus or other cell.

### Fusion Proteins

The disclosure provides fusion proteins comprising an N-terminal targeting sequence linked, directly or indirectly, to at least one molecule of interest (e.g., polypeptide sequence of a protein or peptide of interest, such as at least one plant growth stimulating protein or peptide). In selected embodiments, the indirect linkage may be an intervening spacer, linker or a regulatory sequence. The protein or peptide may comprise, but is not limited to, a peptide hormone, a non-hormone peptide, an enzyme involved in the production or activation of a plant growth stimulating compound or an enzyme that degrades or modifies a bacterial, fungal, or plant nutrient source. In general, any protein of interest capable of expression in a Paenibacillus endospore and heterologous to the selected N-terminal targeting sequence may be used. In some embodiments, the protein of interest is a protein that is expressed in bacteria of the Paenibacillus genus. In other embodiments, the protein of interest is isolated from a bacteria of the same species as the Paenibacillus endospore in which the fusion protein will be expressed. In still other embodiments, the protein of interest is isolated from the Paenibacillus strain in which the fusion protein will be expressed on the endospore. The targeting sequence can be any of the targeting sequences described herein.

In some embodiments, the fusion proteins may comprise a targeting sequence and at least one protein or peptide that protects a plant from a pathogen. The targeting sequence can be any of the targeting sequences described above.

The fusion protein can be made using standard cloning and molecular biology methods known in the art. For example, a gene encoding a protein or peptide (e.g., a gene encoding a plant growth stimulating protein or peptide) can be amplified by polymerase chain reaction (PCR) and ligated to DNA coding for any of the above-described targeting sequences to form a DNA molecule that encodes the fusion protein. The DNA molecule encoding the fusion protein can be cloned into any suitable vector, for example a plasmid vector. The vector suitably comprises a multiple cloning site into which the DNA molecule encoding the fusion protein can be easily inserted. The vector also suitably contains a selectable marker, such as an antibiotic resistance gene, such that bacteria transformed, transfected, or mated with the vector can be readily identified and isolated. Where the vector is a plasmid, the plasmid suitably also comprises an origin of replication. The DNA encoding the fusion protein is suitably under the control of a sporulation promoter that will cause expression of the fusion protein on the spore surface of a Paenibacillus endospore (e.g., a native promoter from a Paenibacillus family member). In some aspects, transcription of the fusion protein is controlled by a promoter present in any of the upstream regulatory sequences described herein (e.g., any of SEQ ID NOs: 11-15, or a fragment or variant thereof which remains transcriptionally active during sporulation). In some aspects, a DNA construct may comprise a sequence encoding a fusion protein according to the disclosure downstream of any of the regulatory sequences described herein (e.g., any of SEQ ID NOs: 11-15), or a fragment or variant thereof which remains transcriptionally active during sporulation. Such fragments may comprise any contiguous 50, 100, 150 or 200 nucleotides of SEQ ID NOs: 11-15, which remain transcriptionally active during sporulation. Similarly, variants may comprise a sequence having at least 50%, 60%, 70%, 80%, or 90% sequence identity compared to any of SEQ ID NOs: 11-15 (or a fragment thereof), which remains transcriptionally active during sporulation. DNA encoding the fusion protein (e.g., a sequence of any of SEQ ID NOs: 1, 3, 5, 7, 9 or a variant or fragment thereof), with one or more upstream regulatory sequence(s) may be integrated into the chromosomal DNA of a Paenibacillus or other cell.

The fusion protein can also comprise additional polypeptide sequences that are not part of the targeting sequence, or the linked protein of interest (e.g., the plant growth stimulating protein or peptide, the protein or peptide that protects a plant from a pathogen, the protein or peptide that enhances stress resistance in a plant, or the plant binding protein or peptide). For example, the fusion protein can include tags or markers to facilitate purification (e.g., a polyhistidine tag) or visualization (e.g., a fluorescent protein such as GFP or YFP) of the fusion protein itself or of the recombinant endospore-producing Paenibacillus cells' spores expressing the fusion protein.

Expression of fusion proteins on the spore surface using the targeting sequences described herein is enhanced due to a lack of secondary structure in the amino-termini of these sequences, which allows for native folding of the fused proteins and retention of activity. Proper folding can be further enhanced by the inclusion of a short amino acid linker between the targeting sequence and the fusion partner protein.

Thus, any of the fusion proteins described herein can comprise an amino acid linker between the targeting sequence and the linked protein of interest (e.g., the plant growth stimulating protein or peptide, the protein or peptide that protects a plant from a pathogen, the protein or peptide that enhances stress resistance in a plant, or the plant binding protein or peptide).

The linker can comprise a polyalanine linker or a polyglycine linker. A linker comprising a mixture of both alanine and glycine residues can also be used. For example, where the targeting sequence comprises SEQ ID NO: 2, a fusion protein can have one of the following structures:

No linker: SEQ ID NO: 2—Fusion Partner Protein

Alanine Linker: SEQ ID NO: 2—Aₙ-Fusion Partner Protein

Glycine Linker: SEQ ID NO: 2—Gₙ-Fusion Partner Protein

Mixed Alanine and Glycine Linker: SEQ ID NO: 2—(A/G)ₙ-Fusion Partner Protein

where Aₙ, Gₙ, and (A/G)ₙ are any number of alanines, any number of glycines, or any number of a mixture of alanines and glycines, respectively.

For example, n can be any integer between 1 to 25, such as an integer between 6 to 10. Where the linker comprises a mixture of alanine and glycine residues, any combination of glycine and alanine residues can be used. The N-terminal targeting sequence represented by SEQ ID NO: 2, as shown above. However, any of the other N-terminal targeting sequences disclosed herein may be substituted in place of SEQ ID NO: 2 (e.g., SEQ ID Nos: 4, 6, 8 or 10 or fragments or variants thereof) in the exemplary configurations above. In the structures shown above, “Fusion Partner Protein” represents the linked protein of interest (e.g., a plant growth stimulating protein or peptide, the protein or peptide that protects a plant from a pathogen, the protein or peptide that enhances stress resistance in a plant, or the plant binding protein or peptide).

Alternatively, or in addition, the linker can comprise a protease recognition site. Inclusion of a protease recognition site allows for targeted removal, upon exposure to a protease that recognizes the protease recognition site, of the protein of interest (e.g., a plant growth stimulating protein or peptide, the protein or peptide that protects a plant from a pathogen, the protein or peptide that enhances stress resistance in a plant, or the plant binding protein or peptide).

In certain aspects, the fusion protein comprises an enzyme involved in the production or activation of a plant growth stimulating compound, such as an acetoin reductase, an indole-3-acetamide hydrolase, a tryptophan monooxygenase, an acetolactate synthetase, an α-acetolactate decarboxylase, a pyruvate decarboxylase, a diacetyl reductase, a butanediol dehydrogenase, an aminotransferase, a tryptophan decarboxylase, an amine oxidase, an indole-3-pyruvate decarboxylase, an indole-3-acetaldehyde dehydrogenase, a tryptophan side chain oxidase, a nitrile hydrolase, a nitrilase, a peptidase, a protease, an adenosine phosphate isopentenyltransferase, a phosphatase, an adenosine kinase, an adenine phosphoribosyltransferase, CYP735A, a 5′-ribonucleotide phosphohydrolase, an adenosine nucleosidase, a zeatin cis-trans isomerase, a zeatin O-glucosyltransferase, a β-glucosidase, a cis-hydroxylase, a CK cis-hydroxylase, a CK N-glucosyltransferase, a 2,5-ribonucleotide phosphohydrolase, an adenosine nucleosidase, a purine nucleoside phosphorylase, a zeatin reductase, a hydroxylamine reductase, a 2-oxoglutarate dioxygenase, a gibberellic 2B/3B hydrolase, a gibberellin 3-oxidase, a gibberellin 20-oxidase, a chitosanase, a chitinase, a β-1,3-glucanase, a β-1,4-glucanase, a β-1,6-glucanase, an aminocyclopropane-1-carboxylic acid deaminase, an enzyme involved in producing a nod factor, or any combination of the above.

In other aspects, the fusion protein comprises an enzyme that degrades or modifies a bacterial, fungal, or plant nutrient source, such as a cellulase, a lipase, a lignin oxidase, a protease, a glycoside hydrolase, a phosphatase, a nitrogenase, a nuclease, an amidase, a nitrate reductase, a nitrite reductase, an amylase, an ammonia oxidase, a ligninase, a glucosidase, a phospholipase, a phytase, a pectinase, a glucanase, a sulfatase, a urease, a xylanase, a siderophore, or any combination of the above.

In some embodiments, the fusion protein is expressed under the control of a sporulation promoter native to the targeting sequence, spore surface protein, or spore surface protein fragment of the fusion protein. The fusion protein may be expressed under the control of a high-expression sporulation promoter. In certain aspects, the high-expression sporulation promoter comprises a sigma-K sporulation-specific polymerase promoter sequence. In selected aspects, the fusion protein may be expressed under the control of a promoter that is native to the targeting sequence of the fusion protein. In some cases, the promoter that is native to the targeting sequence will be a high-expression sporulation promoter. In other cases, the promoter that is native to the targeting sequence will not be a high-expression sporulation promoter. In the latter cases, it may be advantageous to replace the native promoter with a high-expression sporulation promoter. Expression of the fusion protein under the control of a high-expression sporulation promoter provides for increased expression of the fusion protein on the spore surface of the Paenibacillus endospore. The high-expression sporulation promoter can comprise one or more sigma-K sporulation-specific promoter sequences.

As described above, the fusion proteins may comprise a targeting sequence and at least one heterologous protein that may comprise a growth stimulating protein or peptide. The plant growth stimulating protein or peptide can comprise, among other things, a peptide hormone, a non-hormone peptide, an enzyme involved in the production or activation of a plant growth-stimulating compound, or an enzyme that degrades or modifies a bacterial, fungal, or plant nutrient source. The plant growth stimulating protein or peptide can comprise an enzyme involved in the production or activation of a plant growth-stimulating compound. The enzyme involved in the production or activation of a plant growth stimulating compound can be any enzyme that catalyzes any step in a biological synthesis pathway for a compound that stimulates plant growth or alters plant structure, or any enzyme that catalyzes the conversion of an inactive or less active derivative of a compound that stimulates plant growth or alters plant structure into an active or more active form of the compound. Alternatively, the plant growth-stimulating compound can comprise a plant growth hormone, e.g., a cytokinin or a cytokinin derivative, ethylene, an auxin or an auxin derivative, a gibberellic acid or a gibberellic acid derivative, abscisic acid or an abscisic acid derivative, or a jasmonic acid or a jasmonic acid derivative.

Where the enzyme comprises a protease or peptidase, the protease or peptidase can be a protease or peptidase that cleaves proteins, peptides, proproteins, or preproproteins to create a bioactive peptide. The bioactive peptide can be any peptide that exerts a biological activity. The protease or peptidase that cleaves proteins, peptides, proproteins, or preproproteins to create a bioactive peptide can comprise subtilisin, an acid protease, an alkaline protease, a proteinase, an endopeptidase, an exopeptidase, thermolysin, papain, pepsin, trypsin, pronase, a carboxylase, a serine protease, a glutamic protease, an aspartate protease, a cysteine protease, a threonine protease, or a metalloprotease.

The plant growth stimulating protein can also comprise an enzyme that degrades or modifies a bacterial, fungal, or plant nutrient source. Such enzymes include cellulases, lipases, lignin oxidases, proteases, glycoside hydrolases, phosphatases, nitrogenases, nucleases, amidases, nitrate reductases, nitrite reductases, amylases, ammonia oxidases, ligninases, glucosidases, phospholipases, phytases, pectinases, glucanases, sulfatases, ureases, xylanases, and siderophores. When introduced into a plant growth medium or applied to a plant, seed, or an area surrounding a plant or a plant seed, fusion proteins comprising enzymes that degrade or modify a bacterial, fungal, or plant nutrient source can aid in the processing of nutrients in the vicinity of the plant and result in enhanced uptake of nutrients by the plant or by beneficial bacteria or fungi in the vicinity of the plant. The fusion proteins can comprise a targeting sequence and at least one protein or peptide that protects a plant from a pathogen. The protein or peptide can comprise a protein or peptide that stimulates a plant immune response. For example, the protein or peptide that stimulates a plant immune response can comprise a plant immune system enhancer protein or peptide. The plant immune system enhancer protein or peptide can be any protein or peptide that has a beneficial effect on the immune system of a plant. Alternatively, the protein or peptide that protects a plant from a pathogen can be a protein or peptide that has antibacterial activity, antifungal activity, or both antibacterial and antifungal activity. The protein or peptide that protects a plant from a pathogen can also be a protein or peptide that has insecticidal activity, helminthicidal activity, suppresses insect or worm predation, or a combination thereof. The protein that protects a plant from a pathogen can comprise an enzyme. Suitable enzymes include proteases and lactonases. The proteases and lactonases can be specific for a bacterial signaling molecule (e.g., a bacterial lactone homoserine signaling molecule). The enzyme can also be an enzyme that is specific for a cellular component of a bacterium or fungus.

The fusion proteins can comprise a targeting sequence and at least one protein or peptide that enhances stress resistance in a plant. For example, the protein or peptide that enhances stress resistance in a plant comprises an enzyme that degrades a stress-related compound. Stress-related compounds include, but are not limited to, aminocyclopropane-1-carboxylic acid (ACC), reactive oxygen species, nitric oxide, oxylipins, and phenolics. Specific reactive oxygen species include hydroxyl, hydrogen peroxide, oxygen, and superoxide. The enzyme that degrades a stress-related compound can comprise a superoxide dismutase, an oxidase, a catalase, an aminocyclopropane-1-carboxylic acid deaminase, a peroxidase, an antioxidant enzyme, or an antioxidant peptide.

The protein or peptide that enhances stress resistance in a plant can also comprise a protein or peptide that protects a plant from an environmental stress. The environmental stress can comprise, for example, drought, flood, heat, freezing, salt, heavy metals, low pH, high pH, or a combination thereof. For instance, the protein or peptide that protects a plant from an environmental stress can comprise an ice nucleation protein, a prolinase, a phenylalanine ammonia lyase, an isochorismate synthase, an isochorismate pyruvate lyase, or a choline dehydrogenase.

The fusion proteins can comprise a targeting sequence and at least plant binding protein or peptide. The plant binding protein or peptide can be any protein or peptide that is capable of specifically or non-specifically binding to any part of a plant (e.g., a plant root or an aerial portion of a plant such as a leaf, stem, flower, or fruit) or to plant matter. Thus, for example, the plant binding protein or peptide can be a root binding protein or peptide, or a leaf binding protein or peptide.

### Recombinant Paenibacillus Endospores and Cells Expressing the Fusion Proteins

The fusion proteins described herein can be expressed by recombinant endospore-producing Paenibacillus cells (e.g., P. terrae). The fusion protein can be any of the fusion proteins discussed above. The recombinant endospore-producing Paenibacillus cells can co-express two or more of any of the fusion proteins discussed above. For example, the recombinant endospore-producing Paenibacillus cells can co-express at least one fusion protein that comprises a plant binding protein or peptide, together with at least one fusion protein comprising a plant growth stimulating protein or peptide, at least one fusion protein comprising a protein or peptide that protects a plant from a pathogen, or at least one protein or peptide that enhances stress resistance in a plant.

The recombinant endospore-producing Paenibacillus cells may comprise Paenibacillus cells, such as Paenibacillus sp. NRRL B-50972, Paenibacillus terrae, Paenibacillus polymyxa, or Paenibacillus peoriae cells. In other aspects, the endospore-producing Paenibacillus cell may be selected from any of the exemplary Paenibacillus species described herein.

To generate recombinant endospore-producing Paenibacillus cells expressing a fusion protein, any Paenibacillus bacterium may be transformed using standard methods known in the art (e.g., by electroporation with a vector encoding the fusion protein). The bacteria can then be screened to identify transformants by any method known in the art. For example, where the vector includes an antibiotic resistance gene, the bacteria can be screened for antibiotic resistance. Alternatively, DNA encoding the fusion protein can be integrated into the chromosomal DNA of a Paenibacillus cell. The recombinant endospore-producing Paenibacillus cells can then exposed to conditions that will induce sporulation. Suitable conditions for inducing sporulation are known in the art. For example, the recombinant endospore-producing Paenibacillus cells can be plated onto agar plates, and incubated at a temperature of about 30° C. for several days (e.g., 3 days), or alternatively cultured in Schaeffer Sporulation Medium.

Inactivated strains, non-toxic strains, or genetically manipulated strains of any of the above species can also suitably be used. Alternatively or in addition, once the recombinant Paenibacillus family spores expressing the fusion protein have been generated, they can be inactivated to prevent further germination once in use. Any method for inactivating bacterial spores that is known in the art can be used. Suitable methods include, without limitation, heat treatment, gamma irradiation, x-ray irradiation, UV-A irradiation, UV-B irradiation, chemical treatment (e.g., treatment with gluteraldehyde, formaldehyde, hydrogen peroxide, acetic acid, bleach, or any combination thereof), or a combination thereof. Alternatively, spores derived from nontoxigenic strains, or genetically or physically inactivated strains, can be used.

Fusion protein constructs according to the present disclosure comprise an N-terminal signal sequence or a variant or fragment thereof that targets the fusion protein to the spore surface of a Paenibacillus endospore and a polypeptide sequence that is heterologous to the N-terminal signal sequence. In select embodiments, the N-terminal signal sequence and the polypeptide sequence that is heterologous to the N-terminal signal sequence are directly linked. In other aspects, an intervening linker or spacer sequence may be present. In further aspects, a cleavage sequence or other regulatory sequence may be positioned between the two regions. The polypeptide sequence that is heterologous to the N-terminal signal sequence may comprise one or more functional proteins. In aspects where multiple functional proteins are contained in the polypeptide sequence that is heterologous to the N-terminal signal sequence, at least one spacer, cleavage sequence or other regulatory element may be located between the two or more functional proteins.

The polypeptide sequence that is heterologous to the N-terminal signal sequence may be, for example: (a) a plant growth stimulating protein or peptide; (b) a protein or peptide that protects a plant from a pathogen; (c) a protein or peptide that enhances stress resistance of a plant; (d) a plant binding protein or peptide; (e) a plant immune system enhancer protein or peptide; or (f) a protein or peptide that enhances nutrient uptake. When expressed in Paenibacillus, these fusion proteins are targeted to the spore surface of the Paenibacillus endospore and are physically oriented such that the protein or peptide is displayed on the outside of the spore.

This Paenibacillus spore surface display system can be used to deliver peptides, enzymes, and other proteins to plants (e.g., to plant foliage, fruits, flowers, stems, or roots) or to a plant growth medium such as soil. Peptides, enzymes, and proteins delivered to the soil or another plant growth medium in this manner persist and exhibit activity in the soil for extended periods of time. Introduction of recombinant endospore-producing Paenibacillus cells expressing the fusion proteins described herein into soil or the rhizosphere of a plant may lead to a beneficial enhancement of plant growth in many different soil conditions. The use of the Paenibacillus spore surface display system to create these enzymes allows them to continue to exert their beneficial effects on the plant and the rhizosphere over the first months of a plants life, and in some aspects over longer period of time up to and including the life of the plant.

In some aspects, compositions comprising recombinant endospore-producing Paenibacillus cells or endospores produced by such cells according to any aspect described herein may be applied directly to a plant (e.g., as a powder, suspension or solution, to a seed, or to a field). In some aspects, such compositions are applied to a field prior to or after seeding, or alternatively prior to or after sprouting (e.g., pre- or post-planting, or pre- or post-emergence).

In alternative aspects, the fusion proteins and/or compositions disclosed herein may be delivered to a plant, seed, and/or field indirectly by applying recombinant Paenibacillus cells or spores to the plant, seed, or field. In these aspects, a fusion protein may be expressed or generated by the recombinant Paenibacillus cells (e.g. in the field), resulting in delivery of the fusion protein to the plant, seed, or field.

### Recombinant Endospore-Producing Paenibacillus Cells Having Plant-Growth Promoting Effects and/or Other Beneficial Attributes

Some Paenibacillus bacteria are known to have inherent beneficial attributes. For example, some strains have plant-growth promoting or insecticidal (e.g., mosquitocidal) effects. Any of the fusion proteins described herein can be expressed in such strains.

For example, the recombinant endospore-producing Paenibacillus cells may comprise a plant-growth promoting strain of Paenibacillus. The plant-growth promoting strain of bacteria can comprise a strain of bacteria that produces an insecticidal toxin (e.g., a Bin toxin), produces a fungicidal compound (e.g., a β-1,3-glucanase, a chitosinase, a lyticase, or a combination thereof), produces a nematocidal compound (e.g., a Cry toxin), produces a bacteriocidal compound, is resistant to one or more antibiotics, comprises one or more freely replicating plasmids, binds to plant roots, colonizes plant roots, forms biofilms, solubilizes nutrients, secretes organic acids, or any combination thereof.

### Biological Control Agents

Compositions provided by the disclosure may further include biological control agents. Biological control agents can include, in particular, bacteria, fungi or yeasts, protozoa, viruses, entomopathogenic nematodes, inoculants and botanicals and/or mutants of them having all identifying characteristics of the respective strain, and/or at least one metabolite produced by the respective strain that exhibits activity against insects, mites, nematodes and/or phytopathogens. The disclosure provides combinations of the above-described recombinant Paenibacillus endospores with the particular biological control agents described herein and/or to mutants of specific strains of microorganisms described herein, where the mutants have all the identifying characteristics of the respective strain, and/or at least one metabolite produced by the respective strain that exhibits activity against insects, mites, nematodes and/or phytopathogens or promotes plant growth and/or enhances plant health. According to the disclosure, the biological control agents described herein may be employed or used in any physiologic state such as active or dormant

### Exemplary Compositions

In selected aspects, the disclosure provides compositions comprising a) a recombinant endospore-producing Paenibacillus cell that expresses a fusion protein comprising: a targeting sequence that localizes the fusion protein, which comprises a heterologous protein of interest, to the spore surface of a Paenibacillus family member; and b) at least one further and different particular biological control agent disclosed herein and/or a mutant of a specific strain of a microorganism disclosed herein having all identifying characteristics of the respective strain, and/or at least one metabolite produced by the respective strain that exhibits activity against insects, mites, nematodes and/or phytopathogens in a synergistically effective amount. In alternative aspects, the composition comprises at least one additional fungicide and/or at least one insecticide, with the proviso that the recombinant endospore-producing Paenibacillus cells, the insecticide and the fungicide are not identical. In another aspect, composition is used for reducing overall damage of plants and plant parts, as well as, losses in harvested fruits or vegetables caused by insects, mites, nematodes and/or phytopathogens. In another aspect, the composition increases the overall plant health.

The term “plant health” generally comprises various sorts of improvements of plants that are not connected to the control of pests. For example, advantageous properties that may be mentioned are improved crop characteristics including: emergence, crop yields, protein content, oil content, starch content, more developed root system, improved root growth, improved root size maintenance, improved root effectiveness, improved stress tolerance (e.g., against drought, heat, salt, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

Compositions provided by the disclosure may be screened to identify potential benefits to plant growth, health, or other positive attributes by comparing plants which are grown under the same environmental conditions, whereby a part of said plants is treated with a composition according to the present disclosure and another part of said plants is not treated with a composition according to the present disclosure. Instead, said other part is not treated at all or is treated with a suitable control (i.e., an application without a composition according to the disclosure such as an application without all active ingredients), an application without the recombinant endospore-producing Paenibacillus cells as described herein, or an application without a further particular biological control agent disclosed herein.

The composition according to the present disclosure may be applied in any desired manner, such as in the form of a seed coating, soil drench, and/or directly in-furrow and/or as a foliar spray and applied either pre-emergence, post-emergence or both. In other words, the composition can be applied to the seed, the plant or to harvested fruits and vegetables or to the soil wherein the plant is growing or wherein it is desired to grow (plant's locus of growth).

Reducing the overall damage of plants and plant parts often results in healthier plants and/or in an increase in plant vigor and yield. Preferably, the composition according to the present disclosure is used for treating conventional or transgenic plants or seed thereof.

In another aspect, compositions provided by the disclosure improve animal health or the general overall physical condition of such animals. Indicia of enhanced health include one or more of the following: amelioration or reversal of a disease state in an animal; increase in weight gain, which may include an increase in weight of a specific part of the animal or an increase in overall weight; maintenance of gut microflora; increase in feed utilization efficiency; reduction in risk of mortality; increase in disease resistance; reduction in morbidity; increase in immune response; decrease in occurrence of diarrhea, increase in productivity; and/or reduction of pathogen shedding. The present disclosure also relates to methods for improving animal health by administering to an animal a therapeutic or effective amount of any of the compositions described above comprising recombinant endospore-producing Paenibacillus cells that express a fusion protein. In some aspects such fusion protein includes an enzyme that aids in the digestion of feed, such as amylase, glucanase, glucoamylase, cellulase, xylanase, glucanase, and pectinase or an immune modulator, such as an antibody. An effective amount of a composition is an amount effective to enhance the health of an animal in comparison to an animal that has not been administered the composition but otherwise has been administered the same diet (including feed and other compounds) as has the animal receiving the compositions of the present invention. The term “therapeutic amount” refers to an amount sufficient to ameliorate or reverse a disease state in an animal.

In another aspect, compositions provided by the disclosure remove pollution or contaminants from media such as soil, groundwater, sediment or surface water. The present disclosure also relates to methods for removing pollution or contaminants from media such as soil, groundwater, sediment or surface water by applying to such media an effective amount of any of the compositions described above comprising recombinant endospore-producing Paenibacillus cells that express a fusion protein on the spore surface.

### Methods of Using Recombinant Paenibacillus Constructs and Compositions

The present disclosure also relates to methods for stimulating plant growth using any of the compositions described above comprising recombinant endospore-producing Paenibacillus cells that express a fusion protein and at least one of the further particular biological control agents described herein. The method for stimulating plant growth comprises applying to a plant, a seed, a plant part, to the locus surrounding the plant or in which the plant will be planted (e.g., soil or other growth medium) a composition comprising recombinant endospore-producing Paenibacillus cells that express a fusion protein comprising: (i) a heterologous protein (e.g., at least one plant growth stimulating protein); and (ii) a targeting sequence; and at least one further particular biological control agent disclosed herein and/or a mutant of a specific strain of a microorganism disclosed herein having all identifying characteristics of the respective strain, and/or at least one metabolite produced by the respective strain that exhibits activity against insects, mites, nematodes and/or phytopathogens in a synergistically effective amount.

In another aspect of the present disclosure a method for reducing overall damage of plants and plant parts as well as losses in harvested fruits or vegetables caused by insects, mites, nematodes and/or phytopathogens is provided comprising the step of simultaneously or sequentially applying the recombinant endospore-producing Paenibacillus cells and at least one further particular biological control agent described herein in a synergistically effective amount.

In one embodiment of the present method the composition further comprises at least one fungicide. In one aspect, the at least one fungicide is a synthetic fungicide. In another embodiment, the composition comprises at least one insecticide in addition to the fungicide or in place of the fungicide, provided that the insecticide, the fungicide, the recombinant endospore-producing Paenibacillus cells and the particular biological control agent disclosed herein are not identical.

The method of the present disclosure includes the following application methods, namely both of the recombinant endospore-producing Paenibacillus cells and the at least one further particular biological control agent disclosed herein may be formulated into a single, stable composition with an agriculturally acceptable shelf life (so called “solo-formulation”), or being combined before or at the time of use (so called “combined-formulations”).

If not mentioned otherwise, the expression “combination” stands for the various combinations of the recombinant endospore-producing Paenibacillus cells and at least one further particular biological control agent disclosed herein, and optionally at least one fungicide and/or at least one insecticide, in a solo-formulation, in a single “ready-mix” form, in a combined spray mixture composed from solo-formulations, such as a “tank-mix”, and especially in a combined use of the single active ingredients when applied in a sequential manner, i.e., one after the other within a reasonably short period, such as a few hours or days, e.g., 2 hours to 7 days. The order of applying the composition according to the present disclosure is not essential for working the present disclosure. Accordingly, the term “combination” also encompasses the presence of the recombinant endospore-producing Paenibacillus cells and the at least one further particular biological control agent disclosed herein, and optionally at least one fungicide and/or insecticide on or in a plant to be treated or its surrounding, habitat or storage space, e.g., after simultaneously or consecutively applying the recombinant endospore-producing Paenibacillus cells and the at least one further particular biological control agent disclosed herein, and optionally at least one fungicide and/or at least one insecticide to a plant or its surrounding, habitat or storage space.

If the recombinant endospore-producing Paenibacillus cells and the at least one further particular biological control agent described herein, and optionally at least one fungicide and/or at least one insecticide are employed or used in a sequential manner, it is preferred to treat the plants or plant parts (which includes seeds and plants emerging from the seed), harvested fruits and vegetables according to the following method: First, apply at least one fungicide and/or at least one insecticide on the plant or plant parts, and second apply the further particular biological control agent described herein and the recombinant endospore-producing Paenibacillus cells to the same plant or plant parts. By this application manner the amount of residues of insecticides/fungicides on the plant upon harvesting is as low as possible. The time periods between the first and the second application within a (crop) growing cycle may vary and depend on the effect to be achieved. For example, the first application is done to prevent an infestation of the plant or plant parts with insects, mites, nematodes and/or phytopathogens (this is particularly the case when treating seeds) or to combat the infestation with insects, mites, nematodes and/or phytopathogens (this is particularly the case when treating plants and plant parts) and the second application is done to prevent or control the infestation with insects, mites, nematodes and/or phytopathogens and/or to promote plant growth. Control in this context means that the composition comprising the recombinant endospore-producing Paenibacillus cells and the particular biological control agent disclosed herein are not able to fully exterminate the pests or phytopathogenic fungi but are able to keep the infestation on an acceptable level.

The present disclosure also provides methods of enhancing the killing, inhibiting, preventative and/or repelling activity of the compositions of the present disclosure by multiple applications. In some other embodiments, the compositions of the present disclosure are applied to a plant and/or plant part for two times, during any desired development stages or under any predetermined pest pressure, at an interval of about 1 hour, about 5 hours, about 10 hours, about 24 hours, about two days, about 3 days, about 4 days, about 5 days, about 1 week, about 10 days, about two weeks, about three weeks, about 1 month or more. Still in some embodiments, the compositions of the present disclosure are applied to a plant and/or plant part for more than two times, for example, 3 times, 4 times, 5 times, 6 times, 7 times, 8 times, 9 times, 10 times, or more, during any desired development stages or under any predetermined pest pressure, at an interval of about 1 hour, about 5 hours, about 10 hours, about 24 hours, about two days, about 3 days, about 4 days, about 5 days, about 1 week, about 10 days, about two weeks, about three weeks, about 1 month or more. The intervals between each application can vary if it is desired. One skilled in the art will be able to determine the application times and length of interval depending on plant species, plant pest species, and other factors.

By following the before mentioned steps, a very low level of residues of the at least one fungicide and/or at least one insecticide on the treated plant, plant parts, and the harvested fruits and vegetables can be achieved.

If not mentioned otherwise the treatment of plants or plant parts (which includes seeds and plants emerging from the seed), harvested fruits and vegetables with the composition according to the disclosure is carried out directly or by action on their surroundings, habitat or storage space using customary treatment methods, for example dipping, spraying, atomizing, irrigating, evaporating, dusting, fogging, broadcasting, foaming, painting, spreading-on, watering (drenching), drip irrigating. It is furthermore possible to apply the recombinant endospore-producing Paenibacillus cells, the at least one further particular biological control agent described herein, and optionally the at least one fungicide and/or the at least one insecticide as solo-formulation or combined-formulations by the ultra-low volume method, or to inject the composition according to the present disclosure as a composition or as sole-formulations into the soil (in-furrow).

The term “plant to be treated” encompasses every part of a plant including its root system and the material—e.g., soil or nutrition medium—which is in a radius of at least 10 cm, 20 cm, 30 cm around the caulis or bole of a plant to be treated or which is at least 10 cm, 20 cm, 30 cm around the root system of said plant to be treated, respectively.

The amount of the recombinant endospore-producing Paenibacillus cells, which is used or employed in combination with at least one further particular biological control agent described herein, optionally in the presence of at least one fungicide and/or the at least one insecticide, depends on the final formulation as well as size or type of the plant, plant parts, seeds, harvested fruits and vegetables to be treated. Usually, the recombinant endospore-producing Paenibacillus cells to be employed or used according to the disclosure is present in about 1% to about 80% (w/w), preferably in about 1% to about 60% (w/w), more preferably about 10% to about 50% (w/w) of its solo-formulation or combined-formulation with the at least one further particular biological control agent described herein, and optionally the fungicide and/or the at least one insecticide.

Also the amount of the at least one further particular biological control agent disclosed herein which is used or employed in combination with the recombinant endospore-producing Paenibacillus cells, optionally in the presence of at least one fungicide and/or the at least one insecticide, depends on the final formulation as well as size or type of the plant, plant parts, seeds, harvested fruit or vegetable to be treated. Usually, the further particular biological control agent described herein to be employed or used according to the disclosure is present in about 0.1% to about 80% (w/w), preferably 1% to about 60% (w/w), more preferably about 10% to about 50% (w/w) of its solo-formulation or combined-formulation with the recombinant endospore-producing Paenibacillus cells, and optionally the at least one fungicide and/or the at least one insecticide.

Application of the recombinant endospore-producing Paenibacillus cells may be effected as a foliar spray, as a soil treatment, and/or as a seed treatment/dressing. When used as a foliar treatment, in one embodiment, about 1/16 to about 5 gallons of whole broth are applied per acre. When used as a soil treatment, in one embodiment, about 1 to about 5 gallons of whole broth are applied per acre. When used for seed treatment about 1/32 to about ¼ gallons of whole broth are applied per acre. For seed treatment, the end-use formulation contains at least 1×10⁴, at least 1×10⁵, at least 1×10⁶, 1×10⁷, at least 1×x10⁸, at least 1×10⁹, at least 1×10¹⁰ colony forming units per gram.

The ratio can be calculated based on the amount of the at least one further particular biological control agent disclosed herein, at the time point of applying said component of a combination according to the disclosure to a plant or plant part and the amount of the recombinant endospore-producing Paenibacillus cells shortly prior (e.g., 48 h, 24 h, 12 h, 6 h, 2 h, 1 h) or at the time point of applying said component of a combination according to the disclosure to a plant or plant part.

The application of the recombinant endospore-producing Paenibacillus cells and the at least one further particular biological control agent disclosed herein to a plant or a plant part can take place simultaneously or at different times as long as both components are present on or in the plant after the application(s). In cases where the recombinant endospore-producing Paenibacillus cells and further particular biological control agent disclosed herein are applied at different times and the further particular biological control agent disclosed herein is applied prior to the recombinant endospore-producing Paenibacillus cells, the skilled person can determine the concentration of further particular biological control agent disclosed herein on/in a plant by chemical analysis known in the art, at the time point or shortly before the time point of applying the recombinant endospore-producing Paenibacillus cells. Vice versa, when the recombinant endospore-producing Paenibacillus cells are applied to a plant first, the concentration of the recombinant endospore-producing Paenibacillus cells can be determined using tests which are also known in the art, at the time point or shortly before the time point of applying the further particular biological control agent disclosed herein.

In another aspect of the present disclosure a seed treated with the composition as described above is provided. The control of insects, mites, nematodes and/or phytopathogens by treating the seed of plants has been known for a long time and is a subject of continual improvements. Nevertheless, the treatment of seed entails a series of problems which cannot always be solved in a satisfactory manner Thus, it is desirable to develop methods for protecting the seed and the germinating plant that remove the need for, or at least significantly reduce, the additional delivery of crop protection compositions in the course of storage, after sowing or after the emergence of the plants. It is desirable, furthermore, to optimize the amount of active ingredient employed in such a way as to provide the best-possible protection to the seed and the germinating plant from attack by insects, mites, nematodes and/or phytopathogens, but without causing damage to the plant itself by the active ingredient employed. In particular, methods for treating seed ought also to take into consideration the intrinsic insecticidal and/or nematicidal properties of pest-resistant or pest-tolerant transgenic plants, in order to achieve optimum protection of the seed and of the germinating plant with a minimal use of crop protection compositions.

The present disclosure therefore also relates in particular to a method for protecting seed and germinating plants from attack by pests, by treating the seed with the recombinant endospore-producing Paenibacillus cells as defined above and at least one further biological control agent selected from particular microorganisms disclosed herein and/or a mutant of a specific strain of microorganism disclosed herein having all identifying characteristics of the respective strain, and/or at least one metabolite produced by the respective strain that exhibits activity against insects, mites, nematodes and/or phytopathogens and optionally at least one fungicide and/or optionally at least one insecticide of the disclosure. The method of the disclosure for protecting seed and germinating plants from attack by pests encompasses a method in which the seed is treated simultaneously in one operation with the recombinant endospore-producing Paenibacillus cells and the at least one further particular biological control agent described herein, and optionally the at least one fungicide and/or the at least one insecticide. It also encompasses a method in which the seed is treated at different times with the recombinant endospore-producing Paenibacillus cells and the at least one further particular biological control agent disclosed herein, and optionally the at least one fungicide and/or the at least one insecticide.

The disclosure further provides methods of treating seeds for the purpose of protecting the seed and the resultant plant against insects, mites, nematodes and/or phytopathogens. The disclosure also relates to seed which at the same time has been treated with a the recombinant endospore-producing Paenibacillus cells and at least one further particular biological control agent described herein, and optionally at least one fungicide and/or the at least one insecticide. The disclosure further relates to seed which has been treated at different times with the recombinant endospore-producing Paenibacillus cells and the at least one further particular biological control agent disclosed herein and optionally the at least one fungicide and/or the at least one insecticide. In the case of seed which has been treated at different times with the recombinant endospore-producing Paenibacillus cells and the at least one further particular biological control agent disclosed herein, and optionally the at least one fungicide and/or the at least one insecticide, the individual active ingredients in the composition of the disclosure may be present in different layers on the seed.

Furthermore, the disclosure relates to seed which, following treatment with the composition of the disclosure, is subjected to a film-coating process in order to prevent dust abrasion of the seed.

One of the advantages of the present disclosure is that, owing to the particular systemic properties of the compositions of the disclosure, the treatment of the seed with these compositions provides protection from insects, mites, nematodes and/or phytopathogens not only to the seed itself but also to the plants originating from the seed, after they have emerged. In this way, it may not be necessary to treat the crop directly at the time of sowing or shortly thereafter. A further advantage is to be seen in the fact that, through the treatment of the seed with composition of the disclosure, germination and emergence of the treated seed may be promoted.

The compositions of the disclosure are suitable for protecting seed of any variety of plant which is used in agriculture, in greenhouses, in forestry or in horticulture. More particularly, the seed in question is that of cereals (e.g., wheat, barley, rye, oats and millet), maize, cotton, soybeans, rice, potatoes, sunflower, coffee, tobacco, canola, oilseed rape, beets (e.g., sugar beet and fodder beet), peanuts, vegetables (e.g., tomato, cucumber, bean, brassicas, onions and lettuce), fruit plants, lawns and ornamentals. Particularly important is the treatment of the seed of cereals (such as wheat, barley, rye and oats) maize, soybeans, cotton, canola, oilseed rape and rice.

For the purposes of the present disclosure, the composition of the disclosure is applied alone or in a suitable formulation to the seed. The seed is preferably treated in a condition in which its stability is such that no damage occurs in the course of the treatment. Generally speaking, the seed may be treated at any point in time between harvesting and sowing. Typically, seed is used which has been separated from the plant and has had cobs, hulls, stems, husks, hair or pulp removed. Thus, for example, seed may be used that has been harvested, cleaned and dried to a moisture content of less than 15% by weight. Alternatively, seed can also be used that after drying has been treated with water, for example, and then dried again.

When treating seed it is necessary, generally speaking, to ensure that the amount of the composition of the disclosure, and/or of other additives, that is applied to the seed is selected such that the germination of the seed is not adversely affected, and/or that the plant which emerges from the seed is not damaged. This is the case in particular with active ingredients which may exhibit phytotoxic effects at certain application rates.

The compositions of the disclosure can be applied directly, in other words without comprising further components and without having been diluted. As a general rule, it is preferable to apply the compositions in the form of a suitable formulation to the seed. Suitable formulations and methods for seed treatment are known to the skilled person and are described in, for example, the following documents: U.S. Pat. Nos. 4,272,417 A; 4,245,432 A; 4,808,430 A; 5,876,739 A; U.S. Patent Publication No. 2003/0176428 A1; WO 2002/080675 A1; WO 2002/028186 A2, the contents of each of which being incorporated herein by reference.

The combinations which can be used in accordance with the disclosure may be converted into the customary seed-dressing formulations, such as solutions, emulsions, suspensions, powders, foams, slurries or other coating compositions for seed, and also ULV formulations. These formulations are prepared in a known manner, by mixing composition with customary adjuvants, such as, for example, customary extenders and also solvents or diluents, colorants, wetters, dispersants, emulsifiers, antifoams, preservatives, secondary thickeners, stickers, gibberellins, and also water. Colorants which may be present in the seed-dressing formulations which can be used in accordance with the invention include all colorants which are customary for such purposes. In this context it is possible to use not only pigments, which are of low solubility in water, but also water-soluble dyes. Examples include the colorants known under designations Rhodamine B, C.I. Pigment Red 112, and C.I. Solvent Red 1.

Depending on the plant species or plant cultivars, their location and growth conditions (soils, climate, vegetation period, diet), using or employing the composition according to the present disclosure the treatment according to the disclosure may also result in super-additive (“synergistic”) effects. Thus, for example, by using or employing inventive composition in the treatment according to the disclosure, reduced application rates and/or a widening of the activity spectrum and/or an increase in the activity better plant growth, increased tolerance to high or low temperatures, increased tolerance to drought or to water or soil salt content, increased flowering performance, easier harvesting, accelerated maturation, higher harvest yields, bigger fruits, larger plant height, greener leaf color, earlier flowering, higher quality and/or a higher nutritional value of the harvested products, higher sugar concentration within the fruits, better storage stability and/or processability of the harvested products are possible, which exceed the effects which were actually to be expected.

At certain application rates of the inventive composition in the treatment according to the disclosure may also have a strengthening effect in plants. The defense system of the plant against attack by unwanted phytopathogenic fungi and/or microorganisms and/or viruses is mobilized. Plant-strengthening (resistance-inducing) substances are to be understood as meaning, in the present context, those substances or combinations of substances which are capable of stimulating the defense system of plants in such a way that, when subsequently inoculated with unwanted phytopathogenic fungi and/or microorganisms and/or viruses, the treated plants display a substantial degree of resistance to these phytopathogenic fungi and/or microorganisms and/or viruses. Thus, by using or employing composition according to the present disclosure in the treatment according to the disclosure, plants can be protected against attack by the abovementioned pathogens within a certain period of time after the treatment. The period of time within which protection is effected generally extends from 1 to 10 days, preferably 1 to 7 days, after the treatment of the plants with the active compounds.

Any of the compositions disclosed herein may include one or more agrochemicals. Similarly, the methods of applying compositions according to the disclosure may further comprise introducing at least one agrochemical into the plant growth medium or applying at least one agrochemical to plants or seeds.

The agrochemical can comprise a fertilizer (e.g., a liquid fertilizer), a micronutrient fertilizer material (e.g., boric acid, a borate, a boron frit, copper sulfate, a copper frit, a copper chelate, a sodium tetraborate decahydrate, an iron sulfate, an iron oxide, iron ammonium sulfate, an iron frit, an iron chelate, a manganese sulfate, a manganese oxide, a manganese chelate, a manganese chloride, a manganese frit, a sodium molybdate, molybdic acid, a zinc sulfate, a zinc oxide, a zinc carbonate, a zinc frit, zinc phosphate, a zinc chelate, or a combination thereof), an insecticide (e.g., an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or a combination thereof), an herbicide (e.g., a chlorophenoxy compound, a nitrophenolic compound, a nitrocresolic compound, a dipyridyl compound, an acetamide, an aliphatic acid, an anilide, a benzamide, a benzoic acid, a benzoic acid derivative, anisic acid, an anisic acid derivative, a benzonitrile, benzothiadiazinone dioxide, a thiocarbamate, a carbamate, a carbanilate, chloropyridinyl, a cyclohexenone derivative, a dinitroaminobenzene derivative, a fluorodinitrotoluidine compound, isoxazolidinone, nicotinic acid, isopropylamine, an isopropylamine derivatives, oxadiazolinone, a phosphate, a phthalate, a picolinic acid compound, a triazine, a triazole, a uracil, a urea derivative, endothall, sodium chlorate, or a combination thereof), a fungicide (e.g., a substituted benzene, a thiocarbamate, an ethylene bis dithiocarbamate, a thiophthalidamide, a copper compound, an organomercury compound, an organotin compound, a cadmium compound, anilazine, benomyl, cyclohexamide, dodine, etridiazole, iprodione, metlaxyl, thiamimefon, triforine, or a combination thereof), a molluscicide, an algicide, a plant growth amendment, a bacterial inoculant (e.g., a bacterial inoculant of the genus Rhizobium, a bacterial inoculant of the genus Bradyrhizobium, a bacterial inoculant of the genus Mesorhizobium, a bacterial inoculant of the genus Azorhizobium, a bacterial inoculant of the genus Allorhizobium, a bacterial inoculant of the genus Sinorhizobium, a bacterial inoculant of the genus Kluyvera, a bacterial inoculant of the genus Azotobacter, a bacterial inoculant of the genus Pseudomonas, a bacterial inoculant of the genus Azospirillium, a bacterial inoculant of the genus Bacillus, a bacterial inoculant of the genus Streptomyces, a bacterial inoculant of the genus Paenibacillus, a bacterial inoculant of the genus Paracoccus, a bacterial inoculant of the genus Enterobacter, a bacterial inoculant of the genus Alcaligenes, a bacterial inoculant of the genus Mycobacterium, a bacterial inoculant of the genus Trichoderma, a bacterial inoculant of the genus Gliocladium, a bacterial inoculant of the genus Glomus, a bacterial inoculant of the genus Klebsiella, or a combination thereof), a fungal inoculant (e.g., a fungal inoculant of the family Glomeraceae, a fungal inoculant of the family Claroidoglomeraceae, a fungal inoculant of the family Gigasporaceae, a fungal inoculant of the family Acaulosporaceae, a fungal inoculant of the family Sacculosporaceae, a fungal inoculant of the family Entrophosporaceae, a fungal inoculant of the family Pacidsporaceae, a fungal inoculant of the family Diversisporaceae, a fungal inoculant of the family Paraglomeraceae, a fungal inoculant of the family Archaeosporaceae, a fungal inoculant of the family Geosiphonaceae, a fungal inoculant of the family Ambisporaceae, a fungal inoculant of the family Scutellosporaceae, a fungal inoculant of the family Dentiscultataceae, a fungal inoculant of the family Racocetraceae, a fungal inoculant of the phylum Basidiomycota, a fungal inoculant of the phylum Ascomycota, a fungal inoculant of the phylum Zygomycota, or a combination thereof), or a combination thereof.

The fertilizer can comprise ammonium sulfate, ammonium nitrate, ammonium sulfate nitrate, ammonium chloride, ammonium bisulfate, ammonium polysulfide, ammonium thiosulfate, aqueous ammonia, anhydrous ammonia, ammonium polyphosphate, aluminum sulfate, calcium nitrate, calcium ammonium nitrate, calcium sulfate, calcined magnesite, calcitic limestone, calcium oxide, calcium nitrate, dolomitic limestone, hydrated lime, calcium carbonate, diammonium phosphate, monoammonium phosphate, magnesium nitrate, magnesium sulfate, potassium nitrate, potassium chloride, potassium magnesium sulfate, potassium sulfate, sodium nitrates, magnesian limestone, magnesia, urea, urea-formaldehydes, urea ammonium nitrate, sulfur-coated urea, polymer-coated urea, isobutylidene diurea, K₂SO₄—(MgSO₄)₂, kainite, sylvinite, kieserite, Epsom salts, elemental sulfur, marl, ground oyster shells, fish meal, oil cakes, fish manure, blood meal, rock phosphate, super phosphates, slag, bone meal, wood ash, manure, bat guano, peat moss, compost, green sand, cottonseed meal, feather meal, crab meal, fish emulsion, humic acid, or a combination thereof. The agrochemical can comprise any fungicide, bacterial inoculant, or herbicide, as described herein. The spore-forming bacterium, alone or in combination with the insecticide, can further comprise an effective amount of at least one fungicide.

In general, a “fungicide” is a substance to increase mortality or inhibit the growth rate of fungi. The term “fungus” or “fungi” includes a wide variety of nucleated sporebearing organisms that are devoid of chlorophyll. Examples of fungi include yeasts, molds, mildews, rusts, and mushrooms. Typical fungicidal ingredients also include captan, fludioxonil, iprodione, tebuconazole, thiabendazole, azoxystrobin, prochloraz, and oxadixyl. Select compositions, plant seeds, or inoculums according to the disclosure may comprise any natural or synthetic fungicide, such as: aldimorph, ampropylfos, ampropylfos potassium, andoprim, anilazine, azaconazole, azoxystrobin, benalaxyl, benodanil, benomyl, benzamacril, benzamacryl-isobutyl, bialaphos, binapacryl, biphenyl, bitertanol, blasticidin-S, boscalid, bromuconazole, bupirimate, buthiobate, calcium polysulphide, capsimycin, captafol, captan, carbendazim, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, debacarb, dichlorophen, diclobutrazole, diclofluanid, diclomezine, dicloran, diethofencarb, dimethirimol, dimethomorph, dimoxystrobin, diniconazole, diniconazole-M, dinocap, diphenylamine, dipyrithione, ditalimfos, dithianon, dodemorph, dodine, drazoxolon, edifenphos, epoxiconazole, etaconazole, ethirimol, etridiazole, famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluopyram, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, guazatine, hexachlorobenzene, hexaconazole, hymexazole, imazalil, imibenconazole, iminoctadine, iminoctadine albesilate, iminoctadine triacetate, iodocarb, iprobenfos (IBP), iprodione, irumamycin, isoprothiolane, isovaledione, kasugamycin, kresoxim-methyl, copper preparations, such as: copper hydroxide, copper naphthenate, copper oxychloride, copper sulphate, copper oxide, oxine-copper and Bordeaux mixture, mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin, nickel dimethyldithiocarbamate, nitrothal-isopropyl, nuarimol, ofurace, oxadixyl, oxamocarb, oxolinic acid, oxycarboxim, oxyfenthiin, paclobutrazole, pefurazoate, penconazole, pencycuron, phosdiphen, pimaricin, piperalin, polyoxin, polyoxorim, probenazole, prochloraz, procymidone, propamocarb, propanosine-sodium, propiconazole, propineb, prothiocinazole, pyrazophos, pyrifenox, pyrimethanil, pyroquilon, pyroxyfur, quinconazole, quintozene (PCNB), sulphur and sulphur preparations, tebuconazole, tecloftalam, tecnazene, tetcyclasis, tetraconazole, thiabendazole, thicyofen, thifluzamide, thiophanate-methyl, tioxymid, tolclofos-methyl, tolylfluanid, triadimefon, triadimenol, triazbutil, triazoxide, trichlamide, tricyclazole, tridemorph, trifloxystrobin, triflumizole, triforine, uniconazole, validamycin A, vinclozolin, viniconazole, zarilamide, zineb, ziramor, or a combination thereof. The fungicide can also comprise a substituted benzene, a thiocarbamate, an ethylene bis dithiocarbamate, a thiophthalidamide, a copper compound, an organomercury compound, an organotin compound, a cadmium compound, anilazine, benomyl, cyclohexamide, dodine, etridiazole, iprodione, metlaxyl, thiamimefon, triforine, or a combination thereof. One of ordinary skill in the art will readily appreciate that other known synthetic or naturally-occurring fungicides used for agricultural purposes may also be selected for inclusion in a composition, plant seed or inoculum according to the disclosure.

If a composition, plant seed, or inoculum comprises a fungicide, the fungicide can be a foliar fungicide. Foliar fungicides include copper, mancozeb, penthiopyrad, triazoles, cyproconazole, metconazole, propiconazole, prothioconazole, tebuconazole, azoxystrobin, pyraclastobin, fluoxastrobin, picoxystrobin, trifloxystrobin, sulfur, boscalid, thiophanate methyl, chlorothanonil, penthiopyrad, difenconazole, flutriafol, cyprodinil, fluzinam, iprodione, penflufen, cyazofamid, flutolanil, cymoxanil, dimethomorph, pyrimethanil, zoxamide, mandipropamid, metrinam, propamocarb, fenamidone, tetraconazole, chloronab, hymexazol, tolclofos, and fenbuconazole. One of ordinary skill in the art will readily appreciate that other known synthetic or naturally-occurring foliar fungicides used for agricultural purposes may also be selected for inclusion in a composition, plant seed or inoculum according to the disclosure.

Compositions, seeds, and inoculants according to the disclosure comprising an insecticide, possess the ability to increase mortality or inhibit growth rate of insects. As used herein, the term “insects” includes all organisms in the class “Insecta”. The term “pre-adult” insects refers to any form of an organism prior to the adult stage, including, for example, eggs, larvae, and nymphs. As used herein, the terms “insecticide” and “insecticidal” also encompass “nematicide” and “nematicidal” and “acaricide” and “acaricidal.” “Nematicides” and “nematicidal” refers to the ability of a substance to increase mortality or inhibit the growth rate of nematodes. In general, the term “nematode” comprises eggs, larvae, juvenile and mature forms of said organism. “Acaricide” and “acaricidal” refers to the ability of a substance to increase mortality or inhibit growth rate of ectoparasites belonging to the class Arachnida, sub-class Acari.

According to one aspect of the present disclosure, the at least one insecticide comprises:

(1) Acetylcholinesterase (AChE) inhibitors, such as, for example, carbamates, for example alanycarb, bendiocarb, benfuracarb, butocarboxim, butoxycarboxim, carbofuran, carbosulfan, ethiofencarb, furathiocarb, isoprocarb, metolcarb, oxamyl, pirimicarb, propoxur, thiofanox, triazamate, trimethacarb, XMC and xylylcarb; or organophosphates, for example acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, famphur, fenitrothion, fosthiazate, heptenophos, imicyafos, isofenphos, isopropyl O-(methoxyaminothiophosphoryl) salicylate, isoxathion, malathion, mecarbam, methidathion, mevinphos, monocrotophos, naled, omethoate, parathion-methyl, phenthoate, phorate, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, and triclorfon. (2) GABA-gated chloride channel antagonists, such as, for example, cyclodiene-organochlorines, for example chlordane and/or phenylpyrazoles. (3) Sodium channel modulators/voltage-gated sodium channel blockers such as, for example, pyrethroids, e.g., acrinathrin, allethrin, d-cis-trans allethrin, d-trans allethrin, bifenthrin, bioallethrin, bioallethrin s-cyclopentenyl isomer, bioresmethrin, cycloprothrin, cyhalothrin, lambda-cyhalothrin, gamma-cyhalothrin, empenthrin [(EZ)-(IR)-isomer], esfenvalerate, etofenprox, fenpropathrin, fenvalerate, flucythrinate, flumethrin, tau-fluvalinate, halfenprox, imiprothrin, kadethrin, permethrin, phenothrin [(IR)-trans-isomer], prallethrin, pyrethrins (pyrethrum), resmethrin, tefluthrin, tetramethrin, tetramethrin [(IR)-isomer)], and transfluthrin or DDT or methoxychlor. (4) Nicotinergic acetylcholine receptor (nAChR) agonists, such as, for example, neonicotinoids, e.g., dinotefuran, nitenpyram, and thiamethoxam or nicotine or sulfoxaflor. (5) Allosteric activators of the nicotinergic acetylcholine receptor (nAChR) such as, for example, spinosyns, e.g., spinetoram and spinosad. (6) Chloride channel activators, such as, for example, avermectins/milbemycins, for example abamectin, emamectin benzoate, lepimectin and milbemectin. (7) Juvenile hormone imitators such as, for example, juvenile hormone analogues, e.g., hydroprene, kinoprene and methoprene or fenoxycarb or pyriproxyfen. (8) Active compounds with unknown or nonspecific mechanisms of action such as, for example, alkyl halides, e.g., methyl bromide and other alkyl halides; or chloropicrine or sulphuryl fluoride or borax or tartar emetic. (9) Selective antifeedants, for example pymetrozine or flonicamid. (10) Mite growth inhibitors, for example clofentezine, hexythiazox and diflovidazin or etoxazole. (11) Microbial disrupters of the insect gut membrane, for example Bacillus thuringiensis subspecies israelensis, Lysinibacillus sphaericus, Bacillus thuringiensis subspecies aizawai, Bacillus thuringiensis subspecies kurstaki, Bacillus thuringiensis subspecies tenebrionis, and Bt plant proteins: Cry1Ab, Cry1Ac, Cry1Fa, Cry2Ab, mCry3A, Cry3Ab, Cry3Bb, Cry34/35Abl. (12) Oxidative phosphorylation inhibitors, ATP disrupters such as, for example, diafenthiuron or organotin compounds, for example azocyclotin, cyhexatin and fenbutatin oxide or propargite or tetradifon. (13) Oxidative phosphorylation decouplers acting by interrupting the H proton gradient such as, for example, chlorfenapyr, DNOC and sulfluramid. (14) Nicotinergic acetylcholine receptor antagonists such as, for example, bensultap, cartap hydrochloride, thiocylam, and thiosultap-sodium. (15) Chitin biosynthesis inhibitors, type 0, such as, for example, bistrifluron, chlorfluazuron, diflubenzuron, flucycloxuron, flufenoxuron, hexaflumuron, lufenuron, novaluron, noviflumuron, and teflubenzuron. (16) Chitin biosynthesis inhibitors, type 1, for example buprofezin. (17) Moulting inhibitors (in particular for Diptera, i.e., dipterans) such as, for example, cyromazine. (18) Ecdysone receptor agonists such as, for example, chromafenozide, halofenozide, methoxyfenozide and tebufenozide. (19) Octopaminergic agonists. (20) Complex-Ill electron transport inhibitors such as, for example, hydramethylnone or acequinocyl or fluacrypyrim. (21) Complex-I electron transport inhibitors, for example from the group of the METI acaricides, e.g., fenazaquin, fenpyroximate, pyrimidifen, pyridaben, tebufenpyrad and tolfenpyrad or rotenone (Derris). (22) Voltage-gated sodium channel blockers, for example indoxacarb or metaflumizone. (23) Inhibitors of acetyl-CoA carboxylase. (24) Complex-IV electron transport inhibitors such as, for example, phosphines, e.g., aluminium phosphide, calcium phosphide, phosphine and zinc phosphide or cyanide. (25) Complex II electron transport inhibitors, such as, for example, cyenopyrafen and cyflumetofen. (26) Ryanodine receptor effectors, such as, for example, diamides, e.g., chlorantraniliprole, which is also known by the trade name RYNAXYPYR™, and cyantraniliprole, or any combination of one or more of the compounds or classes of compounds identified above.

One of ordinary skill in the art will readily appreciate that other known synthetic or naturally-occurring insecticides used for agricultural purposes may also be selected for inclusion in a composition, plant seed or inoculum according to the disclosure.

### Screening Methods Using the Endospore Display Platforms Described Herein

The fusion protein constructs and recombinant Paenibacillus cells disclosed herein may be used as a platform for high-throughput screening of heterologous proteins that generate new and/or modified plant attributes, as discussed throughout the disclosure. Such attributes may include commercially significant improvements in plant yields and other plant characteristics, such as: altered plant protein or oil content/composition, altered plant carbohydrate content/composition; altered seed carbohydrate content/composition, altered seed oil or protein composition; increased tolerance to environmental or chemical stresses (e.g., resistance to cold or heat, drought, insecticides or herbicides); delayed senescence or disease resistance; growth improvement, health enhancement; herbivore resistance; improved nitrogen fixation or nitrogen utilization; improved root architecture or length; improved water use efficiency; increased biomass; increased seed weight; increased shoot length; increased yield; modified kernel mass or moisture content; metal tolerance; pathogen or pest resistance; photosynthetic capability improvement; salinity tolerance; vigor improvement; increased dry and/or fresh weight of mature seeds, increased number of mature seeds per plant; increased chlorophyll content; a detectable modulation in the level of a metabolite or in the metabolome relative to a reference plant/seed; a detectable modulation in the level of a transcript or in the transcriptome relative to a reference plant/seed; a detectable modulation in the level of a protein or in the proteome relative to a reference plant; and combinations of any of the traits or attributes above. Moreover, the preceding list is intended as a non-limiting set of examples. One of ordinary skill will appreciate that the high-throughput delivery platform disclosed herein is suitable for screening for various other plant traits and attributes discussed elsewhere in the disclosure or otherwise known in the art.

Endospores produced by recombinant Paenibacillus cells modified to express a fusion protein according to the disclosure may be applied to plant cells grown in vitro, a host plant seed, seedling, or to a vegetative or otherwise mature plant. The heterologous protein may in turn modify or confer a trait or attribute to the plant cells grown in vitro, host plant seed, seedling or mature plant. In select embodiments, the Paenibacillus endospores may be used to inoculate a seed and the resulting new or modified trait or attribute may be immediately apparent, whereas on other embodiments it may not become apparent until a later stage of development of the host plant.

In some embodiments, the Paenibacillus bacterium used to deliver the fusion protein is exogenous to the host plant species. In others, the selected Paenibacillus bacterium is an endogenous endophyte known to colonize the host plant species. The host plant may be any suitable plant disclosed here (a monocot, dicot, conifer, etc.)

The recombinant Paenibacillus bacterium used to deliver the fusion protein may be used to inoculate a host plant seed, seedling, vegetative or otherwise mature plant specimen by way of a coating or spray, or any other method of applying endospores to a host plant known in the art. When applied as a liquid, for example, as a solution or suspension, the Paenibacillus endospores may be mixed or suspended in aqueous solutions. Suitable liquid diluents or carriers include aqueous solutions, petroleum distillates, or other liquid carriers. Solid compositions can be prepared by dispersing the Paenibacillus endospores in and on an appropriately divided solid carrier, such as peat, wheat, bran, vermiculite, clay, talc, bentonite, diatomaceous earth, fuller's earth, pasteurized soil, and the like. When such formulations comprise wettable powders, dispersing agents such as non-ionic, anionic, amphoteric, or cationic dispersing and emulsifying agents can be used.

Paenibacillus endospores may be applied directly to the surface of host plant seeds or to the leaves and stem of a vegetative plant directly, or as part of a composition comprising additional components. The additional components may include one or more compounds that enhance the rate of colonization, compounds that enhance plant growth or health, pesticides or herbicides, or any other compounds disclosed herein as suitable for promoting cultivation and growth of plants. Moreover, the composition may include additional Paenibacillus endospores that have been modified to express fusion proteins comprising different amino acid sequences. For example, a composition may comprise a first Paenibacillus endospore that expresses a fusion protein comprising a plant growth promoting factor as well as a second Paenibacillus endospore that expresses a fusion protein that comprises a protein that enhances pesticide-resistance.

In select embodiments, the recombinant Paenibacillus endospore which is coated onto the seed of a host plant is capable, upon germination of the seed into a vegetative state, of localizing to a different tissue of the plant. For example, the recombinant Paenibacillus cells can be capable of localizing to any one of the tissues in the plant, including: the root, adventitious root, seminal root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In other embodiments, the recombinant Paenibacillus cells may be capable of localizing to the root and/or the root hair of the plant. In alternative embodiments, the recombinant Paenibacillus cells may be capable of localizing to the photosynthetic tissues, for example, leaves and shoots of the plant; or to the vascular tissues of the plant, for example, in the xylem and phloem.

In other embodiments, the recombinant Paenibacillus cells are capable of localizing to the reproductive tissues (flower, pollen, pistil, ovaries, stamen, fruit) of the plant. In still another embodiment, the recombinant Paenibacillus cells colonize a fruit or seed tissue of the plant. In still another embodiment, the recombinant Paenibacillus cells are able to colonize the plant such that it is present on the surface of the plant (e.g., the plant exterior or the phyllosphere of the plant). In still other embodiments, the recombinant Paenibacillus cells are capable of localizing to substantially all, or all, tissues of the plant.

Compositions comprising the recombinant Paenibacillus endospores designed for application to a host plant may comprise a seed coating composition, a root treatment, or a foliar application composition. The seed coating composition, or the root treatment, or the foliar application composition may comprise a fungicide, an antibacterial agent, an herbicide, a nematicide, an insecticide, a plant growth regulator, a nutrient, or combinations thereof. The seed coating composition, or the root treatment, or the foliar application composition can further comprise an agriculturally acceptable carrier, a tackifier, a microbial stabilizer, or a combination thereof. In select embodiments, the seed coating composition, or the root treatment, or the foliar application composition can contain a second bacteria, including but not limited to a rhizobial bacterial preparation. The compositions may also contain a surfactant. In one embodiment, the surfactant is present at a concentration of between 0.01% v/v to 10% v/v. In another embodiment, the surfactant is present at a concentration of between 0.1% v/v to 1% v/v. In some embodiments, the composition may include a microbial stabilizer (e.g., a stabilizer).

Upon inoculation, a treated host plant (e.g., a treated seed, seedling, vegetative or otherwise mature plant) may be screened for the existence of new or modified attributes or traits. Screening can occur at any time point following treatment. In select embodiments, a seed may be treated and screening may not occur until the seed has sprouted or reached a more advanced stage of development. In other embodiments, a seed, seedling or vegetative plant may be treated and screening may not occur until the treated plant has produced a harvested end product which may comprise the sample to be screened for a new or modified trait or attribute.

During screening, various tests may be performed both in vitro and in vivo to determine what benefits, if any, are conferred upon the treated host plant. In vivo screening assays include tests that measure phenotypic traits or attributes of a plant or seed (e.g., assays measuring plant growth rate or height; crop yield; resistance to an environmental stress such as heat, cold, or salinity; resistance to biological pathogens or insect pests; resistance to chemical treatments such as insecticides or herbicides). In vitro screening assays include, but are not limited to, tests that measure the composition or properties of plant extracts, tissue samples, cell samples, and the like. In some embodiments, in vitro screening may comprise purifying and measuring the amount or activity of a given protein, enzyme, gene transcript, metabolite or other compound found in the cells or tissue of the treated host plant. In other embodiments, screening may comprise visual inspection of the structure of cells or tissue of the treated host plant, whether by the naked eye or via microscopy.

In alternative embodiments, screening may comprise assays of recombinant Paenibacillus endospores or vegetative cells modified to express a fusion protein according to the present disclosure, as opposed to assays directed to treated host plants. In these embodiments, the Paenibacillus family member cells or endospores may be subject to in vitro assays of one or more activities, such as but not limited to the ability to liberate complexed phosphates or complexed iron (e.g., through secretion of siderophores); production of phytohormones; production of antibacterial, antifungal, or insecticidal, or nematicidal compounds; production and/or secretion of ACC deaminase, acetoin, pectinase, cellulase, or RNase. Screening methods directed to the Paenibacillus family member cells or endospores, rather than vegetative plants, are particularly advantageous in that such methods may allow detection of useful heterologous proteins sooner than methods directed to treated host plants.

### Methods of Identifying Spore Surface Targeting Sequences

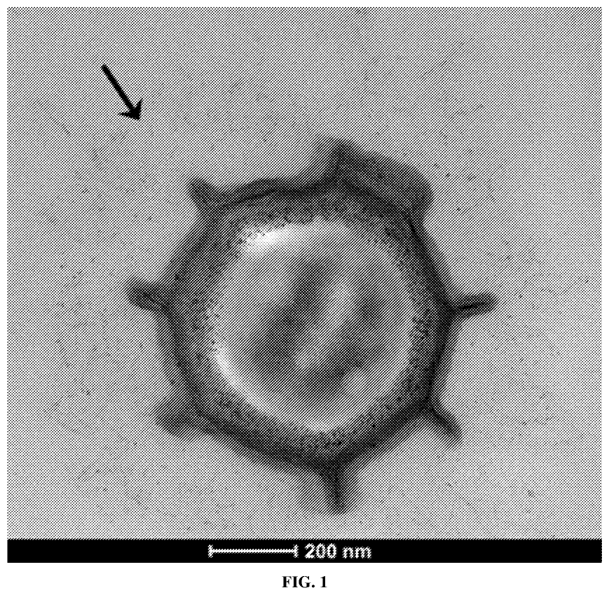

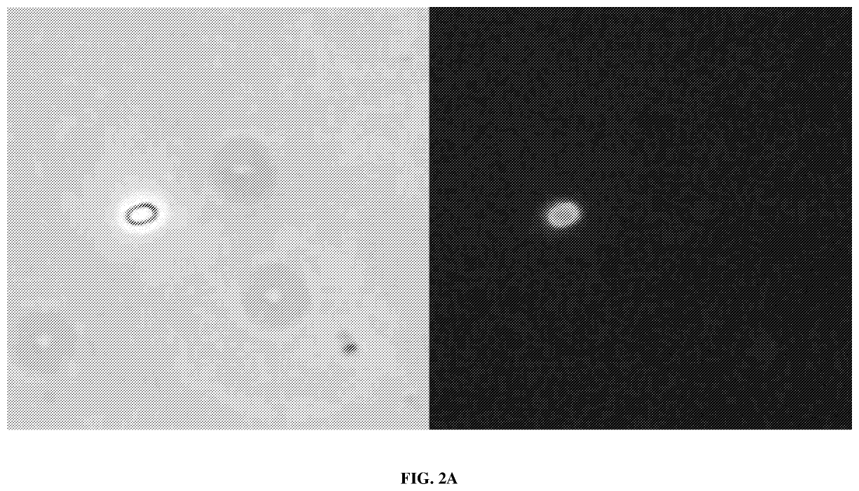

The present disclosure discloses several N-terminal spore surface targeting sequences identified in Paenibacillus, which are useful as part of a spore surface display platform for heterologous proteins as described herein. However, the disclosure is not limited to these particular sequences, fragments and variants thereof. Screening methods according to the disclosure may be broadly used in Paenibacillus and other endospore-forming bacterial genera to identify additional N-terminal spore surface targeting sequences which may be similarly useful as part of an endospore display platform or for other purposes. In one embodiment, the endospore-forming bacterium that are useful for this invention have a hair-like structure that is proteolytically resistant, as shown in FIG. 1. For example, the screening methods disclosed herein may be used to identify N-terminal spore surface targeting sequences in endospore-forming members of Lysinibacillus, Viridibacillus, and Brevibacillus.

In some exemplary aspects, such sequences may be identified by screening a genome of a Paenibacillus or another endospore-forming bacteria of interest for open reading frames (“ORFs”) which encode proteins having multiple collagen-like triplet amino acid repeats of “glycine-any residue-any residue” (“GXX repeats”) and determining that the protein localizes to the spore surface by microscopy or experimentally. These GXX repeats may be adjacent or separate regions of the polypeptide sequence. In some aspects, polypeptide sequences may be screened for a particular number of adjacent or total GXX repeats (e.g., at least 5, 10, 15, 20, 25 or 30 GXX repeats). In some aspects, the protein localization is determined visually (e.g., using transmission electron microscopy) or experimentally (e.g., using mass spectrometry). In some aspects, methods of identifying an N-terminal targeting sequence may further comprise a step of testing the putative N-terminal targeting sequence by expressing a fusion protein comprising the putative N-terminal targeting sequence and a reporter (e.g., GFP) in a Paenibacillus or other bacterial cell.

In some aspects, the disclosure provides spore surface-targeting sequences from Paenibacillus and other bacterial genera (e.g., Lysinibacillus, Viridibacillus, and Brevibacillus) comprising the N-terminal portion of a protein identified via the aforementioned screening process. This N-terminal targeting sequence of such targeting sequences may comprise the first 5, 10, 15, 20, 25, 30, 35, 40, or 50 amino acids of the endogenous sequence, or a fragment or variant thereof. In some aspects, the N-terminal targeting sequence is a variant that is at least 50%, 60%, 70%, 80%, 90% or 95% identical to the endogenous sequence, or a fragment thereof. Spore surface targeting sequences in Paenibacillus and other bacterial genera identified according to these methods may be used to generate heterologous fusion proteins according to any of the various embodiments described herein.

The following non-limiting examples are provided to further illustrate the present disclosure.

### EXAMPLES

### Example 1: General Protocol for Identifying Collagen-Like Spore Surface Proteins Suitable for Endospore Display

The complete genome of Paenibacillus sp. NRRL B-50972 was searched for ORFs containing collagen-like GXX repeats. Collagen-like spore surface proteins were then visualized by transmission electron microscopy (FIG. 1). The presence of collagen-like spore surface proteins was also experimentally confirmed by mass spectrometry. Briefly, Paenibacillus sp. NRRL B-50972 spores were digested with trypsin to remove surface proteins. The spores were removed by centrifugation and the supernatant was analyzed by mass spectrometry to validate the presence of collagen-like spore surface proteins. This general protocol was used to identify endogenous Paenibacillus sp. NRRL B-50972 proteins having the N-terminal targeting sequences identified by SEQ ID NOs: 1-10). The same method may be used to identify spore surface proteins from Viridibacillus, Lysinibacillus or Brevibacillus and corresponding N-terminal targeting sequences.

### Example 2: General Protocol for Preparing Recombinant Paenibacillus Endospores Displaying Green Fluorescent Protein (GFP)

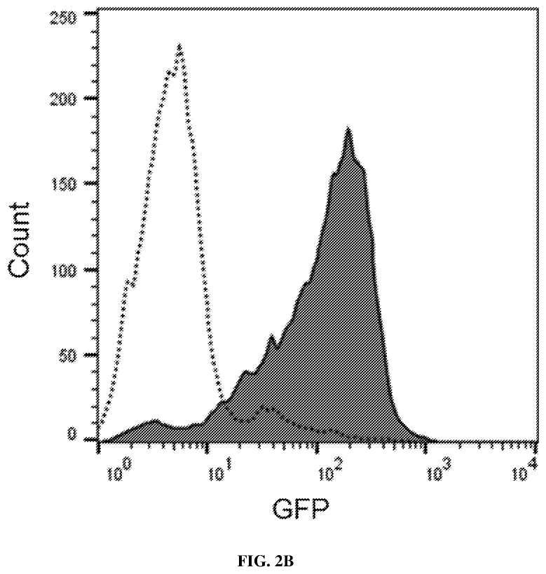

To create fusion constructs, the gene coding for GFP was fused to a DNA segment encoding the amino acids of the disclosed N-terminal targeting sequence (SEQ ID NO: 1) of Paenibacillus sp. NRRL B-50972 under control of the native promoter of the disclosed N-terminal targeting sequences by gene synthesis and cloned into an E. coli/Paenibacillus shuttle vector, pAP13. The resulting vector construct was introduced into Paenibacillus sp. NRRL B-50972. Correct transformants were then grown in Schaeffer's Sporulation Medium broth at 30° C. until sporulation. Paenibacillus sp. NRRL B-50972 spores expressing the fusion construct were then examined by epifluorescent microscopy. GFP is visible on spores expressing the fusion construct (FIG. 2A). Paenibacillus sp. NRRL B-50972 spores were also examined by flow cytometry. Spores expressing the fusion construct are significantly more fluorescent then wild-type spores (FIG. 2B).

### Example 3. General Protocol for Preparing Recombinant Paenibacillus Endospores Displaying an Arbitrary Protein of Interest

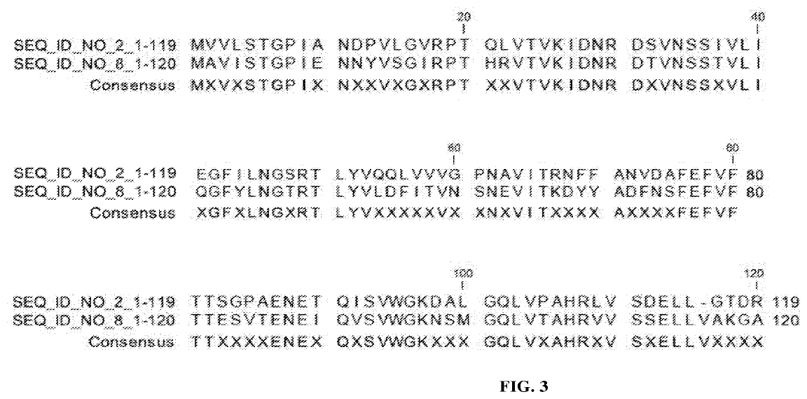

Paenibacillus cells (e.g., Paenibacillus sp. NRRL B-50972) may be cultured, transformed and screened as described above in Example 2 to produce a fusion construct having an N-terminal spore surface targeting sequence according to the disclosure. Screening may proceed by mass spectrometry or any other biochemical or visual means known in the art (e.g., the protein of interest may be tagged with GFP or another selection/screening tag). The N-terminal targeting sequence used to generate the fusion construct may comprise the polypeptide of any of SEQ ID NOs: 2, 4, 6, 8, or 10, 18, 20, 21, 22, 24, 26, 28, 30, or a fragment or variant thereof. In some aspects, the N-terminal targeting sequence may comprise a sequence having one or more residues which correspond to the identical residues in the pairwise alignment of SEQ ID NOs: 2 and 8 (FIG. 3), which is capable of targeting a polypeptide to the spore surface. Similarly, an N-terminal targeting sequence may be used which comprises a sequence having one or more residues which correspond to the identical/conserved residues in the pairwise alignment of SEQ ID NOs: 2 and 8 provided as FIG. 3.

For example, the N-terminal targeting sequence may comprise M-X-V-X-S-T-G-P-I-X-N-X-X-V-X-G-X-R-P-T-X-X-V-T-V-K-I-D-N-R-D-X-V-N-S-S-X-V-L-I-X-G-F-X-L-N-G-X-R-T-L-Y-V-X-X-X-X-X-V-X (SEQ ID NO: 31) (where “X” represents any amino acid), or which comprises any contiguous 5, 10, 15, 20, 25, 30, 35, 40, 45, 50, 55 or 60 residue segment thereof. In another example, the N-terminal targeting sequence may comprise M-X-V-X-S-T-G-P-I-X-N-X-X-V-X-G-X-R-P-T-X-X-V-T-V-K-I-D-N-R-D-X-V-N-S-S-X-V-L-I-X-G-F-X-L-N-G-X-R-T-L-Y-V-X-X-X-X-X-V-X-X-N-X-V-I-T-X-X-X-X-A-X-X-X-X-F-E-F-V-F-T-T-X-X-X-X-E-N-E-X-Q-X-S-V-W-G-K-X-X-X-G-Q-L-V-X-A-H-R-X-V-S-X-E-L- L-V-X-X-X-X (SEQ ID NO: 32) (where “X” represents any amino acid), or which comprises any contiguous 5, 10, 15, 20, 25, 30, 35, 40, 45, 50, 55, 60, 65, 70, 75, 80, 85, 90, 95, 100, 105, 110, 115, or 120 residue segment thereof.

In some aspects, the selected N-terminal targeting sequence may share at least 60%, 65%, 70%, 75%, 80%, 85%, 90% or 95% sequence identity with these sequences and remain capable of targeting the fusion construct to the spore surface.

### Example 4: Methods for Delivering a Fusion Protein Involved in the Production of a Plant Growth Promoting Compound to a Seed, Seedling, Plant, or Plant Part Using Recombinant Paenibacillus Endospores

Enzymes responsible for the production of plant growth promoting compounds can be delivered to plants using the Paenibacillus endospore delivery system disclosed herein. For example, butanediol dehydrogenase converts acetoin to 2,3-butanediol. 2,3-butanediol is a plant growth promoting compound. Paenibacillus endospores expressing this enzyme can be applied as a seed treatment or seed coating or delivered to the area surrounding a seed, seedling, plant, or plant part by drip or spray.

### Example 5: Methods for Delivering Multiple Fusion Proteins on a Single Paenibacillus Endospore to a Seed, Seedling, Plant, or Plant Part Using Recombinant Paenibacillus Endospores

A single recombinant Paenibacillus endospore can be used to display more than one heterologous fusion protein. This is accomplished by constructing two (or more) separate fusion proteins. The coding sequence for each heterologous protein to be displayed on the Paenibacillus endospore surface is fused separately to an N-terminal targeting sequence under control of its native promoters. The fusion protein constructs can be cloned either into the same plasmid vector or different plasmid vectors and introduced into a Paenibacillus member by electroporation. The resulting Paenibacillus endospores will then express a mixture of both heterologous proteins on the spore surface. This is particularly useful for stacking multiple proteinaceous invertebrate toxins to mitigate pest resistance.

### Example 6: Methods for Providing One or More Different Fusion Proteins to a Seed, Seedling, Plant, or Plant Part Using a Combination of Multiple Recombinant Paenibacillus Endospores, Each Displaying One or More Different Fusion Proteins

In certain cases, delivery of more than one Paenibacillus endospore in combination each expressing one or more different heterologous proteins (as described above) are provided. For example, the delivery of nitrogen fixation enzymes to the area surrounding the roots of a plant reduces the need for chemical nitrogen fertilizers. Nitrogen fixation in bacteria may require, at minimum, eight or nine different enzymes and potentially upwards of twenty different enzymes depending on the species. Here, delivery of a combination of Paenibacillus endospores each expressing different enzyme components of the nitrogen fixation pathway may useful. For example, Paenibacillus endospores heterologously displaying NifH, NifD, and NifK may be combined in a mixture with Paenibacillus endospores heterologously displaying NifE, NifN, and NifD and delivered to the area surrounding the roots.

### Example 7: Methods for Delivering an Invertebrate Toxin that Kills Invertebrate Plant Pests to the Area Surrounding a Seed, Seedling, Plant, or Plant Part or as a Seed Treatment Using Recombinant Paenibacillus Endospores

Proteinaceous toxins antagonistic towards invertebrates including but not limited to insects or nematodes can be delivered using the Paenibacillus endospore system. For example, Cry toxins including but not limited to Cry5B and Cry21A which are both insecticidal and nematicidal may be fused to the N-terminal targeting sequence for expression in Paenibacillus endospores. Paenibacillus endospores expressing Cry toxins or other proteinaceous invertebrate toxins can be applied as a seed treatment or seed coating or delivered to the area surrounding a seed, seedling, plant, or plant part by drip or spray for protection against invertebrate plant pathogens.

### Example 8: Methods for Delivering a Peptide, Protein, or Enzyme that is Antagonistic Towards Bacterial Plant Pests to the Area Surrounding a Seed, Seedling, Plant, or Plant Part or as a Seed Treatment Using Paenibacillus Endospores

Bacteriocins are small peptides produced by bacteria with antagonistic activity towards other bacteria. Due to the fact that bacteriocins are ribosomally synthesized as opposed to other antimicrobial molecules (e.g., bacitracin), which are synthesized by large non-ribosomal peptide synthetases, bacteriocins are especially well suited for delivery using the Paenibacillus endospore system. The coding sequence for one or more bacteriocins may be fused to the N-terminal targeting sequence for expression in Paenibacillus endospores. Paenibacillus endospores expressing bacteriocins can be applied as a seed treatment or seed coating or delivered to the area surrounding a seed, seedling, plant, or plant part by drip or spray for protection against bacterial plant pathogens.

### Example 9: Methods for Delivering a Peptide, Protein, or Enzyme that is Antagonistic Towards Fungal Plant Pests to the Area Surrounding a Seed, Seedling, Plant, or Plant Part or as a Seed Treatment Using Paenibacillus Endospores

The primary cell wall component of fungi is chitin. Chitinase is an enzyme that degrades chitin and can be expressed on the surface of Paenibacillus endospores to protect against fungal plant pathogens by destroying their cell walls. Paenibacillus endospores expressing chitinase can be applied as a seed treatment or seed coating or delivered to the area surrounding a seed, seedling, plant, or plant part by drip or spray.

### Example 10: Methods for Delivering an Enzyme that Degrades or Modifies a Bacterial, Fungal, or Plant Nutrient Source to the Area Surrounding a Seed, Seedling, Plant, or Plant Part or as a Seed Treatment Using Paenibacillus Endospores

Enzymes responsible for the degradation or modification of a bacterial, fungal, or plant nutrient source can be delivered to plants using recombinant Paenibacillus endospores. For example, a glycoside hydrolase which breaks down complex polysaccharides can be used to make available simple sugars for beneficial rhizobacteria by treating a plant or seed with recombinant Paenibacillus endospores expressing this (or another) enzyme of interest.

### Example 11: Methods for Assessing Responses to Plant Growth Promoting Biocontrol Agents by Screening of Genomic DNA Libraries Derived from Plant Growth Promoting Biocontrol Agents Using Paenibacillus Endospores

Many of the biocontrol strains used today are recalcitrant to exogenous DNA uptake rendering researchers unable to generate targeted genetic modifications of said strains. Due to this challenge, elucidating the mechanism of action of the plant growth promoting effects of these biocontrol strains is incredibly difficult. Paenibacillus endospores present a novel approach for identifying specific genes responsible for the underlying plant growth promoting effects of biocontrol strains. First, the N-terminal targeting sequence and native promoter are cloned into a suitable E. coli/Bacillus shuttle vector (e.g., pHP13), resulting in a vector suitable for heterologous protein expression on Paenibacillus endospores. All cloning steps and plasmid propagation are performed in E. coli. Next, total gDNA is extracted from a target plant growth promoting biocontrol strain. The gDNA is sheared into fragments (enzymatically or sonically) and ligated into the above described vector for expression of heterologous proteins on Paenibacillus endospores to generate a gDNA library comprised of all the genetic material originating from the biocontrol strain of interest. The resulting vector library is introduced into a Paenibacillus member by electroporation and the bacteria are plated onto agar plates containing an appropriate antibiotic selection agent to select for Paenibacillus endospore transformants. Individual Paenibacillus endospore transformants each expressing a different fragment of the target biocontrol strain's gDNA are assessed for plant growth promoting effects. These effects can include but are not limited to enhanced greening, improved germination, increased plant vigor, increased root length, increased root mass, increased plant height, increased leaf area, or resistance to pests. The vector in Paenibacillus endospore transformants found to modulate the above mentioned plant health parameters can be sequenced to identify the genetic determinants originating from the biocontrol strain responsible for the observed plant growth promoting effects.

### Example 12: Methods for Identifying Novel or Uncharacterized Toxins Antagonistic Against Plant Invertebrate, Bacterial, and Fungal Plant Pathogens Using Paenibacillus Endospores

Many of the biocontrol strains in use today are recalcitrant to exogenous DNA uptake rendering researchers unable to generate targeted genetic modifications of said strains. Due to this challenge, elucidating the mechanism of action by which biocontrol strains are toxic towards invertebrate, bacterial, and fungal plant pathogens is incredibly difficult. Paenibacillus endospores present a novel approach for identifying specific genes responsible for the underlying plant protective effects of biocontrol strains. First, the N-terminal targeting sequence and native promoter are cloned into a suitable E. coli/Bacillus shuttle vector (e.g., pHP13) resulting in a vector suitable for heterologous protein expression on Paenibacillus endospores. All cloning steps and plasmid propagation are performed in E. coli. Next, total gDNA is extracted from a target plant growth promoting biocontrol strain. The gDNA is sheared into fragments (enzymatically or sonically) and ligated into the above described vector for expression of heterologous proteins on Paenibacillus endospores to generate a gDNA library comprised of all the genetic material originating from the biocontrol strain of interest. The resulting vector library is introduced into a Paenibacillus member by electroporation and the bacteria are plated onto agar plates containing an appropriate antibiotic selection agent to select for Paenibacillus endospore transformants. Individual Paenibacillus endospore transformants each expressing a different fragment of the target biocontrol strain's gDNA are assessed for antagonist activity towards invertebrate, bacterial, and fungal plant pathogens. The vector in Paenibacillus endospore transformants that are found to be antagonistic towards the above plant pathogens can be sequenced to identify the genetic determinants originating from the biocontrol strain responsible for the observed plant protective effects.

### Example 13: Methods for Treating a Seed, Seedling, Plant, or Plant Part for the Purposes of Protecting Plants from Pathogens or Improving Plant Health Using Non-Viable Paenibacillus Endospores

There may be a need to deliver plant health promoting proteins/enzymes or plant protection proteins/enzymes using the Paenibacillus endospore delivery system with non-viable (dead) Paenibacillus endospores. Paenibacillus endospores can be inactivated and rendered non-viable via sufficient heat treatment, UV light, gamma irradiation, or high-pressure processing. The resulting non-viable Paenibacillus endospores can be applied as a seed treatment or seed coating or delivered to the area surrounding a seed, seedling, plant, or plant part by drip or spray.

### Example 14. General Protocol for Preparing Recombinant Paenibacillus Endospores Displaying Beta-Galactosidase (B-Gal) from Escherichia coli

To create fusion constructs, the gene coding for β-gal was fused to a DNA segment encoding the amino acids of the disclosed N-terminal targeting sequence (SEQ ID NO: 1) of Paenibacillus sp. NRRL B-50972 under control of the native promoter of the disclosed N-terminal targeting sequences by gene synthesis and cloned into an E. coli/Paenibacillus shuttle vector derived from the pMiniMad vector described in Patrick, J E and Kearns, D B. 2008. MinJ (YvjD) is a Topological Determinant of Cell Division in Bacillus subtilis. Molecular Microbiology. 70: 1166-1179. The resulting vector construct was introduced into a Paenibacillus polymyxa strain (Strain 1) by electroporation similar to that described by Kim and Timmusk (2013), “A Simplified Method for Gene Knockout and Direct Screening of Recombinant Clones for Application in Paenibacillus polymyxa,” PLoSONE, 8(6): e68092, doi: doi:10.1371/journal.pone.0068092. A control was also prepared that contained the shuttle vector without the targeting sequence. Correct transformants were then grown in Schaeffer's Sporulation Medium broth at 30° C. until sporulation. The resulting culture was centrifuged to separate supernatant from spores. Paenibacillus polymyxa spores expressing the fusion construct or containing the empty shuttle vector only and corresponding supernatant were then examined by in vitro assay. β-gal is functional on spores expressing the fusion construct based on hydrolysis of 5-bromo-4-chloro-3-indolyl-β-D-galacto-pyranoside (X-Gal). Results are shown below in Table 3.

TABLE 3
Beta-galactosidase activity of supernatants and spores

| | X-Gal |
|---|---|
| Sample | Hydrolysis^{a} |
| P. polymyxa empy shuttle vector supernatant | − |
| P. polymyxa N-terminal targeting sequence-β- | − |
| galactosidase supernatant | |
| P. polymyxa empy shuttle vector spores | − |
| P. polymyxa N-terminal targeting sequence-β- | + |
| galactosidase-pAP13 spores | |

^{a}X-gal hydrolysis was scored as (−) for no color or (+) for blue color denoting hydrolysis of X-gal by β-galactosidase.

### Example 15. General Protocol for Preparing Recombinant Paenibacillus Endospores Displaying Vegetative Insecticidal Protein 3 (Vip3) from Bacillus thuringiensis (SEQ ID NO: 17)

To create fusion constructs, the gene coding for vip3 (SEQ ID NO: 16) was fused to a DNA segment encoding the amino acids of the disclosed N-terminal targeting sequence (SEQ ID NO: 1) of Paenibacillus sp. NRRL B-50972 by Gibson Assembly into the E. coli/Paenibacillus shuttle vector described in Example 14. Expression of the fusion is under control of the native promoter of the disclosed N-terminal targeting sequence. The resulting vector construct was introduced into a Paenibacillus polymyxa strain (Strain 1) by electroporation, as described above. Correct transformants were then grown in Schaeffer' s Sporulation Medium broth at 30° C. until sporulation.

### Example 16. Activity of the Paenibacillus polymyxa Strain Expressing Vip3 Against Spodoptera exigua

The insecticidal activity of the Paenibacillus polymyxa strain expressing Vip3, from Example 15, was evaluated against Spodotera exigua (beet armyworm). A 96-well plate assay was performed to test the insecticidal activity of each Paenibacillus polymyxa strain including an empty vector control and an active cargo (SEQ ID NO: 2-Vip3). Spores of the strains were produced by growing the strains in Schaeffer' s Sporulation Medium broth until sporulation and centrifuging the resulting whole broth culture to separate spores from supernatant. The spore samples from the strains were then applied to 96-well microplates containing an agar substrate similar to that described in Marrone et al., (1985), “Improvements in Laboratory Rearing of the Southern Corn Rootworm, Diabrotica undecimpuncta howardi Barber (Coleoptera: Chrysomelidae), on an Artificial Diet and Corn,” J. Econ. Entomol., 78: 290-293. The spore samples were then diluted in water and applied at concentrations of 100%, 33%, 11%, 3.7%, and 1.2% to the plates.

After the treatments had been allowed to dry, about 20 eggs from Spodotera exigua (beet armyworm) were added to each well. Several days later, the insecticidal activity was determined by evaluating the stunting scores and mortality scores of the treated larvae. Insect stunting scores were rated according to the following scale: 1=severely stunted; 2=highly stunted, minimal growth; 3=slightly smaller than untreated control; 4=same size as untreated control. The insect mortality score is based on the following scale: 4=0-25% mortality, 3=26-50% mortality, 2=51-79% mortality, 1=80-100% mortality.

Spodotera exigua larvae treated with 11% Paenibacillus spores expressing targeted Vip3 (i.e., SEQ ID NO: 2-Vip3) experienced 2-fold greater stunting thant those treated with the same concentration of Paenibacillus spores expressing the empty vector (see Table 4). Similarly, larvae treated with 11% Paenibacillus spores expressing the targeted Vip3 experienced 1.5-fold greater mortality than those treated with the same concentration of Paenibacillus spores expressing the empty vector (see Table 5).

TABLE 4
Stunting ratings of treated Spodotera exigua (beet armyworm)

| Stunting Score | |
|---|---|
| SEQ ID NO: 2-Vip3 | Empty Vector |

| Application Rate | Mean | Std Err | Mean | Std Err |
|---|---|---|---|---|
| 1 | 1 | 0 | 1 | 0 |
| 0.33 | 1 | 0 | 1.7 | 0.7 |
| 0.11 | 1.5 | 0.5 | 3 | 1 |
| 0.037 | 3.3 | 0.7 | 4 | 0 |
| 0.012 | 4 | 0 | 4 | 0 |

TABLE 5
Mortality ratings of treated Spodotera exigua (beet armyworm)

| Mortality Score | |
|---|---|
| SEQ ID NO: 2-Vip3 | Empty Vector |

| Application Rate | Mean | Std Err | Mean | Std Err |
|---|---|---|---|---|
| 1 | 1 | 0 | 1 | 0 |
| 0.33 | 1 | 0 | 1.3 | 0.3 |
| 0.11 | 1.5 | 0.5 | 2.3 | 0.9 |
| 0.037 | 3.7 | 0.3 | 4 | 0 |
| 0.012 | 4 | 0 | 4 | 0 |

### Example 17. Identification of the Minimal Portion of a Paenibacillus N-terminal Targeting Sequence Necessary for Endospore Display

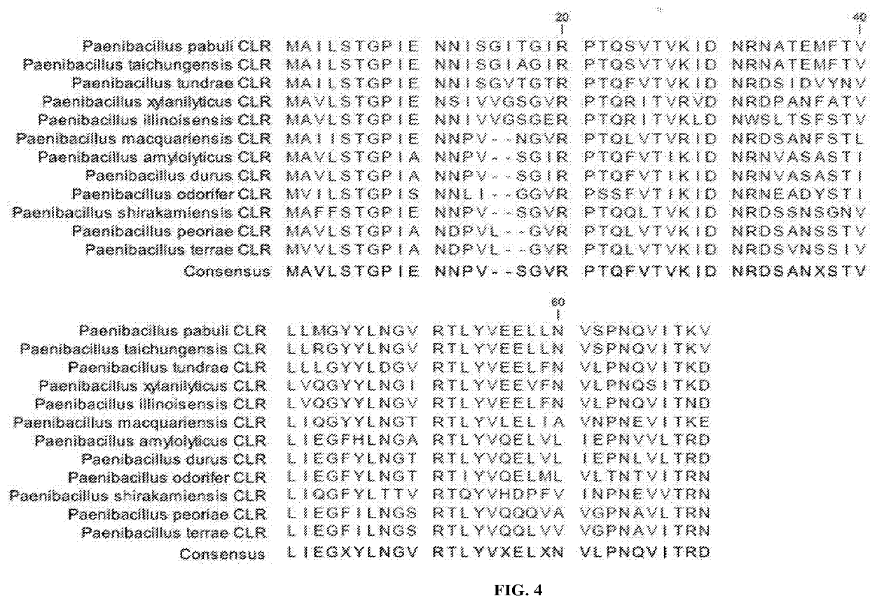

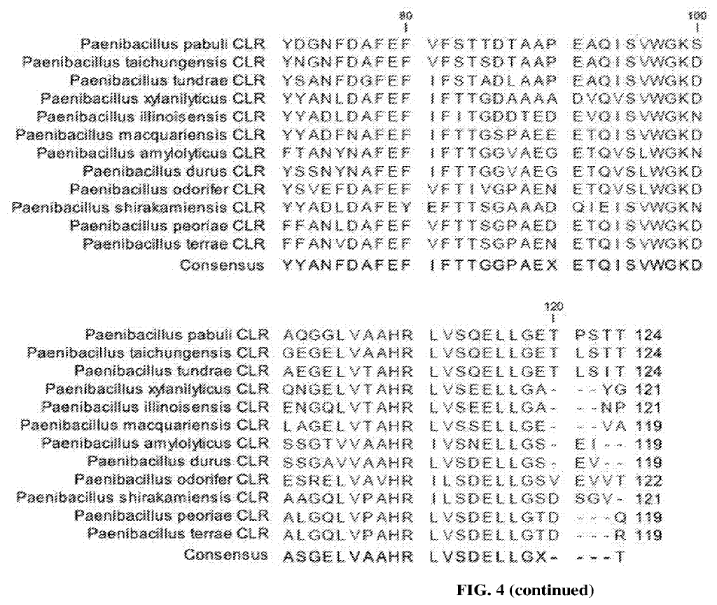

Experiments were conducted to determine which truncated targeting sequences would be sufficient to target a protein of interest to the spore surface. The following general protocol for preparing recombinant Paenibacillus endospores displaying tandem dimer Tomato (tdTom) fluorescent proteins from Discosoma sp. coral. To create fusion constructs, the gene coding for tdTom was fused to a DNA segment encoding the amino acids of the disclosed N-terminal targeting sequence (SEQ ID NO: 2) of Paenibacillus sp. NRRL B-50972 under control of the native promoter of the disclosed N-terminal targeting sequences by gene synthesis and cloned into an E. coli/Paenibacillus shuttle vector (pAP13). Additionally, the gene coding for tdTom was fused to truncations of the N-terminal targeting sequence of SEQ ID NO: 2; namely amino acids 1-100, 1-80, 1-40, 80-100, 90-100, 90-95, 91-98, and 95-100. Truncations 80-100, 90-100, 90-95, 91-98, and 95-100 include an initial methionine for proper translation initiation. Specific sequences of the truncated targeting sequences used in these constructs are provided in Table 8. The resulting vector constructs were introduced into Paenibacillus sp. Strain 1 by electroporation similar to that described by Kim and Timmusk (2013), “A Simplified Method for Gene Knockout and Direct Screening of Recombinant Clones for Application in Paenibacillus polymyxa,” PLoSONE, 8(6): e68092, doi: doi:10.1371/journal.pone.0068092. Correct transformants were then grown in a glucose-based broth at 30° C. until sporulation. Paenibacillus sp. Strain 1 spores expressing the fusion construct were then examined by microscopy. Table 6 provides a summary of constructs in which TdTomato is functional on spores expressing the fusion construct based on detection of fluorescent spores via microscopy. It was observed initially that the 1-100 truncation worked well, while the 1-80 and 1-40 truncations did not. Applicant then tested the 80-100 truncation, which also provided fluorescence. Finally, Applicant analyzed the collagen-like repeat region corresponding to SEQ ID NO: 2 in over a hundred Paenibacillus strains, to determine whether a consensus sequence existed among these targeting sequences. FIG. 4 provides a sampling of the strains that were analyzed and the resulting consensus sequence from amino acids 91-98 of the targeting sequence. This consensus sequence, with an initial methionine, is provided as SEQ ID NO: 49 in Table 6, below. Experimental work confirmed that use of this consensus sequence, with an aspartic acid as amino acid residue 98, caused fluorescence on the spore surface. Constructs with a shorter targeting sequence did not fluoresce.

Paenibacillus sp. Strain 1 spores were also examined by flow cytometry with antibody staining. Spores expressing tdTom fused to full-length, 1-100, and 91-98 targeting sequences are significantly more fluorescent then wild-type spores.

TABLE 6
SEQ ID NO: 2 truncation series used to identify the minimal
domain required for spore surface targeting activity

| | Fluorescent |
|---|---|
| Targeting Sequence | Spores |
| SEQ ID NO: 2 (Full length (1-119) | + |
| Amino Acids 1-100 from SEQ ID NO: 2 | + |
| Amino Acids 1-40 from SEQ ID NO: 2 | − |
| Amino Acids 1-80 from SEQ ID NO: 2 | − |
| Amino Acids 80-100 from SEQ ID NO: 2 | + |
| Amino Acids 90-100 from SEQ ID NO: 2 | + |
| Amino Acids 90-95 from SEQ ID NO: 2 | − |
| Amino Acids 91-98 from SEQ ID NO: 2 | + |
| Amino Acids 95-100 from SEQ ID NO: 2 | − |

### Example 18. Results of a β-galactosidase Activity Assay of Selected Truncation Mutants

Experiments were conducted to determine whether certain truncated targeting sequences would be sufficient to target an enzyme of interest to the spore surface. Following is the general protocol for preparing recombinant Paenibacillus endospores displaying beta-galactosidase β-gal) from Escherichia coli. To create fusion constructs, the gene coding for β-gal was fused to a DNA segment encoding amino acids 80-100 of the N-terminal targeting sequence of Paenibacillus sp. NRRL B-50972, which is SEQ ID NO: 2, with an added initial methionine, under control of the native promoter of the disclosed N-terminal targeting sequences by gene synthesis and cloned into an E. coli/Paenibacillus shuttle vector (pAP13). The DNA segment encoding amino acids 80-100 with an added initial methionine is SEQ ID NO: 40 in Table 8, below. The resulting vector construct was introduced into Paenibacillus sp. Strain 1 by electroporation similar to that described above. Correct transformants were then grown in a glucose-based broth at 30° C. until sporulation. Paenibacillus sp. Strain 1 spores expressing the fusion construct were then examined by in vitro assay. The 80-100 truncation displayed a functional β-galactosidase.

TABLE 7
β-galactosidase assay results

| | X-Gal |
|---|---|
| Sample | Hydrolysis^{a} |
| Strain 1 pAP13 supernatant | − |
| Strain 1 N-terminal targeting seq-β-galactosidase- | − |
| pAP13 supernatant | |
| Strain 1 pAP13 spores | − |
| Strain 1 N-terminal targeting seq-β-galactosidase- | + |
| pAP13 spores | |
| Strain 1 N-terminal targeting seq(80-100)-β- | − |
| galactosidase supernatant | |
| Strain 1 N-terminal targeting seq(80-100)-β- | + |
| galactosidase spores | |

^{a}X-gal hydrolysis was scored as (−) for no color or (+) for blue color denoting hydrolysis of X-gal by β-galactosidase

TABLE 8
Truncated N-terminal targeting sequence
constructs analyzed in Examples 17 and 18

| Sequence Identifier | Sequence |
|---|---|
| N-terminal 1-100 | ATGGTAGTATTATCTACTGGACCTATTGCAAACGATCCTGTTCTAGG |
| (nucleotide) | AGTCAGACCCACCCAACTGGTCACAGTAAAAATAGATAACCGAGATT |
| SEQ ID NO: 33) | CTGTAAATTCTTCTATCGTTTTGATCGAGGGTTTTATTTTAAACGGT |
| | AGCAGAACATTATATGTACAACAATTAGTGGTAGTGGGACCAAATGC |
| | GGTTATAACGAGGAATTTCTTTGCAAATGTAGACGCATTTGAATTCG |
| | TTTTTACCACTAGCGGACCAGCAGAGAATGAAACTCAAATTTCTGTT |
| | TGGGGTAAAGATGCATTG |
| N-terminal 1-100 | MVVLSTGPIANDPVLGVRPTQLVTVKIDNRDSVNSSIVLIEGFILNG |
| (polypeptide) | SRTLYVQQLVVVGPNAVITRNFFANVDAFEFVFTTSGPAENETQISV |
| SEQ ID NO: 34 | WGKDAL |
| N-terminal 1-80 | ATGGTAGTATTATCTACTGGACCTATTGCAAACGATCCTGTTCTAGG |
| (nucleotide) | AGTCAGACCCACCCAACTGGTCACAGTAAAAATAGATAACCGAGATT |
| SEQ ID NO: 35 | CTGTAAATTCTTCTATCGTTTTGATCGAGGGTTTTATTTTAAACGGT |
| | AGCAGAACATTATATGTACAACAATTAGTGGTAGTGGGACCAAATGC |
| | GGTTATAACGAGGAATTTCTTTGCAAATGTAGACGCATTTGAATTCG |
| | TTTTT |
| N-terminal 1-80 | MVVLSTGPIANDPVLGVRPTQLVTVKIDNRDSVNSSIVLIEGFILNG |
| (polypeptide) | SRTLYVQQLVVVGPNAVITRNFFANVDAFEFVF |
| SEQ ID NO: 36 | |
| N-terminaal 1-40 | ATGGTAGTATTATCTACTGGACCTATTGCAAACGATCCTGTTCTAGG |
| (nucleotide) | AGTCAGACCCACCCAACTGGTCACAGTAAAAATAGATAACCGAGATT |
| SEQ ID NO: 37 | CTGTAAATTCTTCTATCGTTTTGATC |
| N-terminal 1-40 | MVVLSTGPIANDPVLGVRPTQLVTVKIDNRDSVNSSIVLI |
| (polypeptide) | |
| SEQ ID NO: 38 | |
| N-terminal 80-100 | ATGTTTACCACTAGCGGACCAGCAGAGAATGAAACTCAAATTTCTGT |
| (nucleotide) | TTGGGGTAAAGATGCATTG |
| SEQ ID NO: 39 | |
| N-terminal 80-100 | MFTTSGPAENETQISVWGKDAL |
| (polypeptide) | |
| SEQ ID NO: 40 | |
| N-terminal 90-100 | ATGACTCAAATTTCTGTTTGGGGTAAAGATGCATTG |
| (nucleotide) | |
| SEQ ID NO: 41 | |
| N-terminal 90-100 | MTQISVWGKDAL |
| (polypeptide) | |
| SEQ ID NO: 42 | |
| N-terminal 90-95 | ATGACTCAAATTTCTGTTTGG |
| (nucleotide) | |
| SEQ ID NO: 43 | |
| N-terminal 90-95 | MTQISVW |
| (polypeptide) | |
| SEQ ID NO: 44 | |
| N-terminal 91-98 | ATGCAAATTTCTGTTTGGGGTAAAGAT |
| (nucleotide) | |
| SEQ ID NO: 45 | |
| N-terminal 91-98 | MQISVWGKD |
| (polypeptide) | |
| SEQ ID NO: 46 | |
| N-terminal 95-100 | ATGTGGGGTAAAGATGCATTG |
| (nucleotide) | |
| SEQ ID NO: 47 | |
| N-terminal 95-100 | MWGKDAL |
| (polypeptide) | |
| SEQ ID NO: 48 | |
| Consensus Sequence | MQISVWGK(D/N) |
| (polypeptide) | |
| SEQ ID NO: 49 | |

Unless defined otherwise, all technical and scientific terms herein have the same meaning as commonly understood by one of ordinary skill in the art to which this invention belongs. All publications, patents, and patent publications cited are incorporated by reference herein in their entirety for all purposes.

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. A nucleic acid molecule encoding a fusion protein, comprising (a) a first polynucleotide sequence encoding an N-terminal signal peptide, operably linked to (b) a second polynucleotide sequence encoding a polypeptide heterologous to the N-terminal signal peptide, wherein the first polynucleotide sequence comprises:
(i) SEQ ID NOs: 39, 41, or 45;
(ii) a polynucleotide sequence encoding an N-terminal signal peptide having at least 50%, 60%, 70%, 80%, 90%, 95%, or 98% sequence identity with any of SEQ ID NOs: 39, 41, or 45;
(iii) a polynucleotide sequence encoding one or more of the following polypeptide sequences: “MFTTSGPAENETQISVWGKDAL” (SEQ ID NO: 40), “MTQISVWGKDAL” (SEQ ID NO: 42), or “MQISVWGK(D/N)” (SEQ ID NO: 49); or
(iv) a polynucleotide sequence encoding a polypeptide sequence of at most 20, 30, or 40 amino acids, wherein the polypeptide sequence comprises: “MFTTSGPAENETQISV WGKDAL” (SEQ ID NO: 40), “MTQISVWGKDAL” (SEQ ID NO: 42), or “MQISVWGK(D/N)” (SEQ ID NO: 49);
wherein the N-terminal signal peptide is capable of targeting the fusion protein to a spore surface of a Paenibacillus endospore.

2. The nucleic acid molecule of claim 1, wherein the first polynucleotide sequence consists of or consists essentially of a sequence encoding the polypeptide sequence:
“MFTTSGPAENETQISVWGKDAL” (SEQ ID NO: 40), “MTQISVWGKDAL” (SEQ ID NO:
42), or “MQISVWGK(D/N)” (SEQ ID NO: 49).

3. (canceled)

4. The nucleic acid molecule of claim 1, wherein the first polynucleotide sequence consists essentially of a sequence encoding amino acids 80-90, 90-100, or 91-98 of SEQ ID NO: 2 or amino acids 2-9 of SEQ ID NO: 49.

5. The nucleic acid molecule of claim 1, wherein the polypeptide heterologous to the N-terminal signal peptide comprises:
(a) a plant growth-stimulating protein;
(b) an enzyme;
(c) a protein;
(d) a polypeptide heterologous to Paenibacillus; or
(e) a plant immune-stimulating protein.

6. The nucleic acid molecule of claim 1, further comprising a third polynucleotide sequence, encoding:
(a) a polypeptide comprising one or more protease cleavage sites, wherein the polypeptide is positioned between the N-terminal signal peptide and the polypeptide heterologous to the N-terminal signal peptide;
(b) a polypeptide comprising a selectable marker;
(c) a polypeptide comprising a visualization marker;
(d) a polypeptide comprising a protein recognition/purification domain; or
(e) a polypeptide comprising a flexible linker element, which connects the N-terminal signal peptide and the polypeptide heterologous to the N-terminal signal peptide.

7. The nucleic acid molecule of claim 1, wherein the Paenibacillus endospore is an endospore formed by a Paenibacillus species, comprising: Paenibacillus sp. NRRL B-50972, Paenibacillus terrae, Paenibacillus polymyxa, or Paenibacillus peoriae; or
an endospore formed by a bacterium that possesses a 16S rRNA gene that shares at least 97, 98 or 99% identity with a 16S rRNA gene of a Paenibacillus species.

8. The nucleic acid molecule of claim 1, operatively linked to a promoter element that is heterologous to at least one of the second polynucleotide sequences and Paenibacillus.

9. The nucleic acid molecule of claim 1, wherein the first polynucleotide sequence comprises:
a codon-optimized polynucleotide sequence having at least 50%, 60%, 70%, 80% or 90% sequence identity with SEQ ID NOs: 39, 41, or 45, or a fragment thereof, which is expressed at a higher rate or level in the Paenibacillus endospore compared to the corresponding unoptimized sequence under identical conditions.

10. A fusion protein comprising an N-terminal signal peptide operably linked to a polypeptide heterologous to the N-terminal signal peptide, wherein the N-terminal signal peptide comprises:
(i) a polypeptide comprising an amino acid sequence of SEQ ID NOs: 40, 42, 46 or 49;
(ii) a polypeptide consisting of an amino acid sequence of SEQ ID NOs: 40, 42, 46 or 49;
(iii) a polypeptide comprising or consisting of an amino acid sequence having at least 50%, 55%, 60%, 65%, 70%, 75%, 80%, 85%, 90%, 95%, or 98% sequence identity with the amino acid sequence of any one of SEQ ID NOs: 40, 42, 46 or 49; or
(iv) a polypeptide comprising or consisting of a fragment of at least 7 consecutive amino acids of any one of SEQ ID NOs: 40, 42, 46 or 49;
wherein the N-terminal signal peptide is capable of targeting the fusion protein to the spore surface of a Paenibacillus endospore.

11. The fusion protein of claim 10, wherein the N-terminal signal peptide consists of at most 10, 15, 20, 25, 30, or 35 amino acids.

12. The fusion protein of claim 10, wherein the polypeptide heterologous to the N-terminal signal peptide comprises:
(a) a plant growth-stimulating protein;
(b) an enzyme;
(c) a protein;
(d) a polypeptide heterologous to Paenibacillus;
(e) a therapeutic protein; or
(f) a plant immune-stimulating protein.

13. The fusion protein of claim 10, wherein the fusion protein further comprises:
(a) a polypeptide containing one or more protease cleavage sites, positioned between the N-terminal signal peptide and the polypeptide heterologous to the N-terminal signal peptide;
(b) a polypeptide comprising a selectable marker;
(c) a polypeptide comprising a visualization marker;
(d) a polypeptide comprising at least one protein recognition/purification domain; or
(e) a polypeptide comprising a flexible linker element, connecting the signal peptide and the polypeptide heterologous to the N-terminal signal peptide.

14. The fusion protein of claim 10, wherein the Paenibacillus endospore is an endospore formed by a Paenibacillus species, comprising: Paenibacillus sp. NRRL B-50972, Paenibacillus terrae, Paenibacillus polymyxa, or Paenibacillus peoriae; or an endospore formed by a bacterium that possesses a 16S rRNA gene that shares at least 97, 98 or 99% identity with a 16S rRNA gene of a Paenibacillus species.

15. A recombinant Paenibacillus cell comprising a bacterial chromosome comprising the nucleic acid molecule of claim 1.

16. A vector comprising the nucleic acid molecule of claim 1, wherein the vector comprises a plasmid, an artificial chromosome, or a viral vector.

17. The vector of claim 16, further comprising at least one of the following:
(a) an origin of replication that provides stable maintenance in a Paenibacillus cell;
(b) an origin of replication that provides selectively non-stable maintenance in a Paenibacillus cell;
(c) a temperature-sensitive origin of replication that provides selectively non-stable maintenance in a Paenibacillus cell;
(d) a polynucleotide encoding a selection marker, operably linked to an expression control sequence; or
(e) a polynucleotide encoding a plant growth stimulating protein, operably linked to an expression control sequence.

18. A recombinant Paenibacillus cell transformed with a vector comprising the nucleic acid molecule of claim 1.

19. The recombinant Paenibacillus cell of claim 18, wherein the Paenibacillus cell is a Paenibacillus species, comprising: Paenibacillus sp. NRRL B-50972, Paenibacillus terrae, Paenibacillus polymyxa, or Paenibacillus peoriae; or a bacterium that possesses a 16S rRNA gene that shares at least 97, 98 or 99% identity with a 16S rRNA gene of a Paenibacillus species.

20-21. (canceled)

22. A seed treated with the recombinant bacterial cell of claim 18.

23-35. (canceled)


SEQUENCE LISTING (49 sequences)

SEQ ID NO: 1 - DNA, 357 bp, Paenibacillus sp. NRRL B-50972
atggtagtattatctactggacctattgcaaacgatcctgttctaggagtcagacccacccaactggtcacagtaaaaatagataaccgagattctgtaaattcttctatcgttttgatcgagggttttattttaaacggtagcagaacattatatgtacaacaattagtggtagtgggaccaaatgcggttataacgaggaatttctttgcaaatgtagacgcatttgaattcgtttttaccactagcggaccagcagagaatgaaactcaaatttctgtttggggtaaagatgcattggggcaattagtacctgcccatcggttagtatctgacgaacttttaggaaccgatcga

SEQ ID NO: 2 - protein, 119 aa, Paenibacillus sp. NRRL B-50972
MVVLSTGPIANDPVLGVRPTQLVTVKIDNRDSVNSSIVLIEGFILNGSRTLYVQQLVVVGPNAVITRNFFANVDAFEFVFTTSGPAENETQISVWGKDALGQLVPAHRLVSDELLGTDR

SEQ ID NO: 3 - DNA, 231 bp, Paenibacillus sp. NRRL B-50972
atgcctgccttggatgaatggagtagtatacaacaaatcgatatggaggtgtttgtattgggtcgtcccgaattgaaacgaaagaaaggccgtaaaaaagacgtttttatccgctcttggtttagtaaaaaacgtccgaagagaaaatgccattcgaaacgaaagtgcttttgcaaggaaatcgtcgtcagaaagcaaatcgtccgtgtaaatatacctcaaaatgtttta

SEQ ID NO: 4 - protein, 77 aa, Paenibacillus sp. NRRL B-50972
MPALDEWSSIQQIDMEVFVLGRPELKRKKGRKKDVFIRSWFSKKRPKRKCHSKRKCFCKEIVVRKQIVRVNIPQNVL

SEQ ID NO: 5 - DNA, 438 bp, Paenibacillus sp. NRRL B-50972
atgaaacacagaaaaccgttcaggttcagtggtgcttcaaaaaaagacgaggactgcaaaccacctaaaattagcagagaaacggaagaacttctcaaactgattaaggaattagtcgccatcatcccgctcgttttcgcaaacccgtctgtggctaatgtaacttcattgcaacagattttacagcgattattagctctcgcaaataaattgagacttagaggctcggctaagacagatttattagcggcgttggaactggctatcgtggcgtcggaagccactcttttctccccgatcggtgttggaacgacactgcaacaactgctggaagtcttattgtctattattttgcaggaaccccttgatcctgctcttaaagacagtttgatcagtgcaatcagaaatgccgaaacggctatcagtattgcgttgggt

SEQ ID NO: 6 - protein, 146 aa, Paenibacillus sp. NRRL B-50972
MKHRKPFRFSGASKKDEDCKPPKISRETEELLKLIKELVAIIPLVFANPSVANVTSLQQILQRLLALANKLRLRGSAKTDLLAALELAIVASEATLFSPIGVGTTLQQLLEVLLSIILQEPLDPALKDSLISAIRNAETAISIALG

SEQ ID NO: 7 - DNA, 360 bp, Paenibacillus sp. NRRL B-50972
atggcggttatatcaactggacccatagaaaataattatgtcagtggtattcggcctactcatcgagttaccgtgaaaattgataatcgtgatactgtgaattcttctacggtattgattcagggtttttatctaaatggtacaagaacgttatatgtgcttgattttataactgtaaattcaaatgaagtgattacaaaagattattatgctgattttaattcatttgagtttgtttttaccactgaaagtgttacagaaaatgagattcaagtttcagtctggggtaaaaattcaatggggcagttagtgacagctcaccgtgttgtatcttccgaattgcttgtagcaaaaggcgcg

SEQ ID NO: 8 - protein, 120 aa, Paenibacillus sp. NRRL B-50972
MAVISTGPIENNYVSGIRPTHRVTVKIDNRDTVNSSTVLIQGFYLNGTRTLYVLDFITVNSNEVITKDYYADFNSFEFVFTTESVTENEIQVSVWGKNSMGQLVTAHRVVSSELLVAKGA

SEQ ID NO: 9 - DNA, 330 bp, Paenibacillus sp. NRRL B-50972
ttgggaaatttattgttgcgtaaaagatatcgcttgacccaggtggcaaggaaaaaaaagaaggaaagagatcaaaagatgggagcgttccgttttatgcccatttatcgtacaggaacgagctgcattcgtaacaaaaagggaaataaacgtatttatagacagggtagaagaagagagagaatatgcgcttatagacatcatttgcacgctgagcgggtgccctcaggtttatcaaataaaaaaatctgttttatgaaattcaaaggtcaacgaagactgcgaggcggcgaacaggagcctcaaggcaattcaggaggagcagttcaa

SEQ ID NO: 10 - protein, 110 aa, Paenibacillus sp. NRRL B-50972
LGNLLLRKRYRLTQVARKKKKERDQKMGAFRFMPIYRTGTSCIRNKKGNKRIYRQGRRRERICAYRHHLHAERVPSGLSNKKICFMKFKGQRRLRGGEQEPQGNSGGAVQ

SEQ ID NO: 11 - DNA, 200 bp, Paenibacillus sp. NRRL B-50972
gaaacgggagtggtgaaatcattgatgctcagcgcattgttgcggatgagcaactagattcttgaaacacaacatatgtacagagatagaaccacaatcgtaacaaatggttgagacataaaatagagggaacaggatcttgagaaagatctcattgttcacaaaaaagcttgattttactagaaaggagggagtatcca

SEQ ID NO: 12 - DNA, 200 bp, Paenibacillus sp. NRRL B-50972
ctatatacatgcgcaaaaaacggcttcaaactgcttcataattacggcacgtttcttctggcgccttcggctgttccttggtgtgaaccaaggtaacagccgggggcgctatttttatataactagatgaatgtacctgtacaaagacccatttttatccaaaattagatcattgcctatcaaccacaggacagatgtcc

SEQ ID NO: 13 - DNA, 200 bp, Paenibacillus sp. NRRL B-50972
agcgttacaagttggaagcccggtttggaaatacagaaaatcgatattaaagcttatgtacaagcatccaataataattcttgtgtggtgattcacccttttcgcttcagtaaatatattgttaatatctgcgaaacggggcgatgatccacctgtcacctctacagtagggagaaatgtgaaggaggagatatttgaac

SEQ ID NO: 14 - DNA, 200 bp, Paenibacillus sp. NRRL B-50972
agcggtattttttgtgccccacaaaaaaggctcccttatcaaaaggggtttttatcacataggaaatgtccacacgtatatatagatgttacatattatataaatcgtgaacattcgaatctcaatactagttatagaagaggtggcattagtgataggattatagcttcgttactttagacaaaaggagaatccaatat

SEQ ID NO: 15 - DNA, 200 bp, Paenibacillus sp. NRRL B-50972
atttatttttttgaaaaattacaggggattcagtcccactttcagtaaattcagaaagaaaaataatgtaacggcgaaatggaagtgagcattaaaaatttatttttttggaaaaaaatttaaggaggtcatctgtccaatcaggttcgtttagattccataagataatgaaactgtacttaattatggaggtgtcagta

SEQ ID NO: 16 - DNA, 2370 bp, Bacillus thuringiensis
atgaacaaaaacaacactaaattatctactcgtgctcttccatctttcattgactacttcaacggtatttacggtttcgctacaggtatcaaagacattatgaacatgatcttcaaaactgatactggtggtgacttaactcttgatgaaattcttaagaaccaacaattacttaacgacatctctggtaaacttgatggtgttaacggttctcttaacgacttaatcgcacaaggtaaccttaacactgaactaagcaaagaaatcctaaaaattgctaatgaacaaaaccaagttcttaatgatgttaacaacaaactagacgctattaacactatgttacgtgtataccttccaaaaatcacttcaatgctttctgatgttatgaaacaaaactacgctttatctttacaaatcgaatacttatctaagcaattacaggaaatctctgataaattagacatcatcaacgttaacgtattaattaacagcactcttacagaaattacgccagcttaccaacgtatcaaatacgttaatgaaaaattcgaagaacttacattcgctactgaaacttcttctaaagttaaaaaagacggttctcctgctgatattttagatgaattaactgaattaactgaacttgctaaatctgtaactaaaaacgatgttgacggtttcgaattctaccttaacactttccacgatgtaatggtaggcaacaaccttttcggtcgctctgctcttaaaactgctagtgaattaatcactaaagaaaacgttaaaacttctggtagcgaagtaggtaacgtttacaatttcttaatcgtactaacagctcttcaagcacaagcattccttactcttactacttgccgtaaattacttggtttagctgatattgattacacttctatcatgaacgaacatcttaacaaagaaaaagaagaattccgtgttaatatccttccgacattatctaacactttttctaacccaaattacgctaaggttaaaggtagcgatgaagatgcaaaaatgatcgtagaagctaaaccaggtcacgcattaatcggtttcgagatctcaaacgacagcatcacagttttaaaagtttatgaagctaaacttaaacaaaactaccaagtagataaagacagcctgtctgaagttatctacggtgacatggataaattattatgtccagatcaaagtgaacaaatctactacactaataacatcgtattcccaaacgaatatgtaatcactaaaatcgatttcacaaagaaaatgaaaactcttcgttacgaggtaactgctaacttttacgattcatctactggtgaaattgatcttaacaagaaaaaagttgaatcttctgaagctgaataccgtacattatctgctaacgatgatggtgtttatatgcctttaggtgtaatttctgaaacattcttaactcctatcaacggtttcggtcttcaagctgatgaaaactctcgcttaatcactttaacatgcaaatcttaccttcgcgaacttttattagctacagatttatctaacaaagaaactaaacttattgttcctccatctggattcattagcaatatcgttgagaacggtagcattgaagaagataaccttgaaccttggaaagctaacaacaaaaacgcatacgttgatcatacaggtggtgttaacggtacaaaagctctttacgtacacaaagatggtggtattagccaattcatcggcgacaaattaaagccaaaaactgaatacgttatccaatacactgtaaaaggtaaaccatcaattcatttaaaagatgaaaatactggttacattcattacgaagacactaacaacaaccttgaagattaccaaacaatcaacaaacgttttacaacaggaactgacttaaaaggtgtttacttaattttaaaatctcaaaacggtgacgaggcatggggcgacaacttcatcattctagaaatttctccttctgaaaaattactttctcctgaattaatcaatactaacaactggacttctacaggttctactaacatttctggaaacactttaacactttaccaaggtggtcgtggtatcttaaaacaaaacttacaattagattcattctctacataccgtgtttatttctctgtatctggtgacgctaacgtacgtatccgtaactctcgtgaagtattatttgaaaaacgttacatgtcaggagctaaagatgtatctgaaatgttcactactaaattcgaaaaagataatttctacattgaattatctcaaggtaacaacctttacggtggtccaatcgttcacttctacgatgtttctatcaaataa

SEQ ID NO: 17 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 18 - protein, 110 aa, Paenibacillus sp. NRRL B-50972
MGNLLLRKRYRLTQVARKKKKERDQKMGAFRFMPIYRTGTSCIRNKKGNKRIYRQGRRRERICAYRHHLHAERVPSGLSNKKICFMKFKGQRRLRGGEQEPQGNSGGAVQ

SEQ ID NO: 19 - DNA, 3582 bp, Paenibacillus sp. NRRL B-50972
atggtagtattatctactggacctattgcaaacgatcctgttctaggagtcagacccacccaactggtcacagtaaaaatagataaccgagattctgtaaattcttctatcgttttgatcgagggttttattttaaacggtagcagaacattatatgtacaacaattagtggtagtgggaccaaatgcggttataacgaggaatttctttgcaaatgtagacgcatttgaattcgtttttaccactagcggaccagcagagaatgaaactcaaatttctgtttggggtaaagatgcattggggcaattagtacctgcccatcggttagtatctgacgaacttttaggaaccgatcgaggaatccaaggacctcaaggagttcagggagcccaaggcgaccaaggtgaccaaggacctcagggtgttcaaggacctcaaggagttcagggagcccaaggagaccaaggagttcaaggcgtacaaggagaccaaggacctcaaggagtccaaggcgaccaaggtgaccaaggacctcaaggagttcaaggagcgcaaggtgaccaaggccctcaaggagttcagggagcccaaggtgaccaaggacctcaaggcgttcagggagcgcaaggtgaccaaggacctcaaggtgatcaaggacctcagggagttcaaggagaccaaggcgatcaaggaccacagggagttcaaggcgtacaaggtgatcaaggacctcagggtgttcaaggagaccaaggcgaccaaggacctcagggtgttcaaggcgtacaaggtgaccaaggacctcagggtgttcaaggcgtacaaggtgaccaaggacctcagggagttcaaggagaccaaggcgatcaaggaccacagggagttcaaggcgtacaaggtgatcaaggacctcagggtgttcaaggagaccaaggcgaccaaggacctcagggtgttcaaggcgtacaaggtgaccaaggacctcagggtgttcaaggcgtacaaggtgaccaaggacctcagggtgttcaaggcgtacaaggtgaccaaggacctcaaggagttcagggagcccaaggtgaccaaggaccacagggagttcaaggcgaccaaggacctcaaggacctcaaggagttcaaggtgaccaaggacctcagggcgttcaaggatcccaaggtgatcaaggacctcaaggagttcaaggcgtacaaggacctcaaggagttcaaggcgtacaaggcgaccaaggacctcaaggtgttcagggagcccaaggcgaccaaggccctcaaggagttcaaggagtccaaggtgaccaaggaccacagggagttcaaggaccgcaaggtgaccaaggaccacagggagttcagggagtccaaggcgaccaaggacctcaaggagtccaaggcgaccaaggtgaccaaggacctcaaggagttcaaggagcgcaaggtgaccaaggccctcaaggagttcagggagcccaaggtgaccaaggacctcaaggcgttcagggagcgcaaggtgaccaaggacctcaaggtgatcaaggacctcagggagttcaaggagaccaaggcgatcaaggaccacagggagttcaaggcgtacaaggtgatcaaggacctcagggtgttcaaggagaccaaggcgaccaaggacctcagggtgttcaaggcgtacaaggtgaccaaggacctcagggtgttcaaggcgtacaaggtgaccaaggacctcagggtgttcaaggcgtacaaggtgaccaaggacctcaaggagttcagggagcccaaggtgaccaaggaccacagggagttcaaggcgaccaaggacctcaaggacctcaaggagttcaaggtgaccaaggacctcagggcgttcaaggatcccaaggtgatcaaggacctcaaggagttcaaggcgtacaaggacctcaaggagttcaaggcgtacaaggcgaccaaggacctcaaggtgttcagggagcccaaggcgaccaaggccctcaaggagttcaaggagtccaaggtgaccaaggaccacagggagttcaaggaccgcaaggtgaccaaggaccacagggagttcagggagtccaaggcgaccaaggacctcaaggtgaccaaggacctcaaggtgaccaaggacctcaaggtgttcaaggtgaccaaggacctcaaggagttcagggagcccaaggcgaccaaggacctcaaggagttcaaggaccgcaaggtgaccaaggacctcaaggagttcaaggcgtacaaggtgatcaaggacctcaaggagttcaaggcgtacaaggtgaccaaggaccacagggtgttcaaggcgtacaaggtgaccaaggacctcaaggtgttcaaggagtccaaggtgatcaaggacctcaaggagttcagggagcccaaggcgaccaaggacctcagggagttcagggagcccaaggcgaccaaggacctcagggagttcagggagcccaaggtgaccaaggacctcagggcgttcaaggagtacaaggtgaccaaggatctcaaggagttcaaggaccgcaaggtgaccaaggacctcaaggagttcaaggcgtacaaggtgaccaaggacctcaaggagttcaaggagtccaaggtgaccaaggacctcaaggtgttcagggagcccaaggtggccaaggacctcagggcgttcaaggcgaccaaggtgaccaaggaccacagggtgttcaaggatctcaaggtgaccaaggaccacaaggcgttcaaggagcccaaggcgaccaaggaccacagggtgttcaaggcgtacaaggtgaccaaggccctcaaggagttcaaggagttcaaggtgaccaaggaccacagggagttcaaggtgttcaaggaccgcaaggtgaccaaggaccacagggtgttcaaggagcccaaggcgaccaaggaccacagggtgttcaaggagtgcaaggtgaccaaggaccgcaaggcgaccaaggtgaccaaggacctcaaggtgttcagggagtccaaggcgaccaaggacctcaaggtgttcagggagtccaaggcgaccaaggacctcaaggtgttcaaggtgaccaaggaccacagggagttcagggagcccaaggtgaccaaggacctcagggagttcaaggtgaccaaggtgaccaaggacctcaaggagttcaaggtgtacaaggtgaccaaggacctcaaggtgttcagggagcccaaggtgaccaaggacctcagggcgtacaaggcgaccaaggtgaccaaggaccacagggtgttcaaggcgtacaaggtgatcaaggacctcaaggagttcaaggcgtacaaggtgaccaaggaccacagggtgttcaaggcgtacaaggtgaccaaggaccacagggtgttcaaggcgaccaaggtgaccaaggacctcaaggcgtacaaggcgatcaaggacctcagggtgttcaaggacctcagggtgttcaaggacctcagggtgttcaaggacctcaaggcgaccaaggagctcaaggtgttcaaggaccacaaggtgaccaaggaccgcaaggcattctgtaa

SEQ ID NO: 20 - protein, 1193 aa, Paenibacillus sp. NRRL B-50972
MVVLSTGPIANDPVLGVRPTQLVTVKIDNRDSVNSSIVLIEGFILNGSRTLYVQQLVVVGPNAVITRNFFANVDAFEFVFTTSGPAENETQISVWGKDALGQLVPAHRLVSDELLGTDRGIQGPQGVQGAQGDQGDQGPQGVQGPQGVQGAQGDQGVQGVQGDQGPQGVQGDQGDQGPQGVQGAQGDQGPQGVQGAQGDQGPQGVQGAQGDQGPQGDQGPQGVQGDQGDQGPQGVQGVQGDQGPQGVQGDQGDQGPQGVQGVQGDQGPQGVQGVQGDQGPQGVQGDQGDQGPQGVQGVQGDQGPQGVQGDQGDQGPQGVQGVQGDQGPQGVQGVQGDQGPQGVQGVQGDQGPQGVQGAQGDQGPQGVQGDQGPQGPQGVQGDQGPQGVQGSQGDQGPQGVQGVQGPQGVQGVQGDQGPQGVQGAQGDQGPQGVQGVQGDQGPQGVQGPQGDQGPQGVQGVQGDQGPQGVQGDQGDQGPQGVQGAQGDQGPQGVQGAQGDQGPQGVQGAQGDQGPQGDQGPQGVQGDQGDQGPQGVQGVQGDQGPQGVQGDQGDQGPQGVQGVQGDQGPQGVQGVQGDQGPQGVQGVQGDQGPQGVQGAQGDQGPQGVQGDQGPQGPQGVQGDQGPQGVQGSQGDQGPQGVQGVQGPQGVQGVQGDQGPQGVQGAQGDQGPQGVQGVQGDQGPQGVQGPQGDQGPQGVQGVQGDQGPQGDQGPQGDQGPQGVQGDQGPQGVQGAQGDQGPQGVQGPQGDQGPQGVQGVQGDQGPQGVQGVQGDQGPQGVQGVQGDQGPQGVQGVQGDQGPQGVQGAQGDQGPQGVQGAQGDQGPQGVQGAQGDQGPQGVQGVQGDQGSQGVQGPQGDQGPQGVQGVQGDQGPQGVQGVQGDQGPQGVQGAQGGQGPQGVQGDQGDQGPQGVQGSQGDQGPQGVQGAQGDQGPQGVQGVQGDQGPQGVQGVQGDQGPQGVQGVQGPQGDQGPQGVQGAQGDQGPQGVQGVQGDQGPQGDQGDQGPQGVQGVQGDQGPQGVQGVQGDQGPQGVQGDQGPQGVQGAQGDQGPQGVQGDQGDQGPQGVQGVQGDQGPQGVQGAQGDQGPQGVQGDQGDQGPQGVQGVQGDQGPQGVQGVQGDQGPQGVQGVQGDQGPQGVQGDQGDQGPQGVQGDQGPQGVQGPQGVQGPQGVQGPQGDQGAQGVQGPQGDQGPQGIL

SEQ ID NO: 21 - protein, 572 aa, Paenibacillus sp. NRRL B-50972
MVVLSTGPIANDPVLGVRPTQLVTVKIDNRDSVNSSIVLIEGFILNGSRTLYVQQLVVVGPNAVITRNFFANVDAFEFVFTTSGPAENETQISVWGKDALGQLVPAHRLVSDELLGTDRGIQGPQGVQGAQGDQGDQGPQGVQGPQGVQGAQGDQGVQGVQGDQGPQGVQGDQGDQGPQGVQGAQGDQGPQGVQGAQGDQGPQGVQGAQGDQGPQGDQGPQGVQGDQGDQGPQGVQGVQGDQGPQGVQGDQGDQGPQGVQGVQGDQGPQGVQGVQGDQGPQGVQGVQGDQGPQGVQGAQGDQGPQGVQGDQGPQGLQGVQGDQGPQGVQGSQGDQGPQGVQGVQGPQGGQGVQGDQGPQGVQGAQGDQGPQGVQGDQGDQGPQGVQGAQGDQGPQGVQGDQGDQGPQGVQGDQGTKELKEYKVTKELKEFKEPKVTKDLRAFKATKVTKDHRVFKEFKVTKDLKEFKEYKVTKDHRVFKAYKVTKDLKVFKATKVTKDLKAYKAIKDLRVFKDLRVFKDLRVFKDLKATKELKVFKDHKVTKDRKAFCKLKVKVYLDDSKVIITFGSFFVLS

SEQ ID NO: 22 - protein, 450 aa, Paenibacillus sp. NRRL B-50972
MVVLSTGPIANDPVLGVRPTQLVTVKIDNRDSVNSSIVLIEGFILNGSRTLYVQQLVVVGPNAVITRNFFANVDAFEFVFTTSGPAENETQISVWGKDALGQLVPAHRLVSDELLGTDRGIQGPQGVQGAQGDQGDQGPQGVQGPQGVQGAQGDQGVQGVQGDQGPQGVQGDQGDQGPQGVQGAQGDQGPQGVQGAQGDQGPQGVQGAQGDQGPQGDQGPQGVQGDQGDQGPQGVQGVQGDQGPQGVQGDQGDQGPQGVQGVQGDQGPQGVQGVQGDQGPQGVQGVQGDQGPQGVQGAQGDQGPQGVQGDQGPQGPQGVQGDQGPQGVQGAQGDQGPQGVQGVQGPQGVQGVQGDQGPQGVQGAQGDQGPQGVQGVQGDQGPQGVQGAQGDQGPQGVQGVQGDQGPQGVQGDQGDQGPQGVQGAQGDQGPQGVQGAQGDQGPQGVQGAQG

SEQ ID NO: 23 - DNA, 1032 bp, Paenibacillus sp. NRRL B-50972
atgcctgccttggatgaatggagtagtatacaacaaatcgatatggaggtgtttgtattgggtcgtcccgaattgaaacgaaagaaaggccgtaaaaaagacgtttttatccgctcttggtttagtaaaaaacgtccgaagagaaaatgccattcgaaacgaaagtgcttttgcaaggaaatcgtcgtcagaaagcaaatcgtccgtgtaaatatacctcaaaatgttttaggtataacaggcgcaactggagctataggtgtagcaggtaacgtaggtgcagcgggcactgtgggtgctgctggagccgtcggaactgcgggaaatgtcggggctgccggtaatgtgggtactgcgggcaccgttgggactgccggaaatgtaggcgcagcgggggctgtgggcactgcgggcgctgttggagctgcgggtgcggtaggaccagtaggtcccgtaggtcctgcgggcattccaggggcagtcggtccagcaggtcctgcgggcgttgcaggggcggtcggtcctgtaggtcctgcgggtgcggtaggtgccactggggctacgggtaccgcaggagcgacggggtccaccggggctacgggagctacaggaaccgcaggtggaatagctcagtttggttatatctacaacttaggatcccgagtcgttccaatagaagcggatgtcattttcgatacgaacggtatacttacacctggaattacccacgctcccggcactacgcagattgcagttaccgatgcggggaactatgaagttaacttttcagtatcgggtgtagagccaggccaatttgccatatttatcaatggcactctggcagcaggaaccatatacggctcaggagctggtacgcagcaaaacacagggcaggccatcctcgctctagcatccggtgatgttcttaccctgcgaaatcatagctctgccgctgcggttaccctgcaaaccttggctggaggtacccaagccaacgtaaacgcttctgtcgttatcaaaaaattaagttag

SEQ ID NO: 24 - protein, 343 aa, Paenibacillus sp. NRRL B-50972
MPALDEWSSIQQIDMEVFVLGRPELKRKKGRKKDVFIRSWFSKKRPKRKCHSKRKCFCKEIVVRKQIVRVNIPQNVLGITGATGAIGVAGNVGAAGTVGAAGAVGTAGNVGAAGNVGTAGTVGTAGNVGAAGAVGTAGAVGAAGAVGPVGPVGPAGIPGAVGPAGPAGVAGAVGPVGPAGAVGATGATGTAGATGSTGATGATGTAGGIAQFGYIYNLGSRVVPIEADVIFDTNGILTPGITHAPGTTQIAVTDAGNYEVNFSVSGVEPGQFAIFINGTLAAGTIYGSGAGTQQNTGQAILALASGDVLTLRNHSSAAAVTLQTLAGGTQANVNASVVIKKLS

SEQ ID NO: 25 - DNA, 1497 bp, Paenibacillus sp. NRRL B-50972
atgaaacacagaaaaccgttcaggttcagtggtgcttcaaaaaaagacgaggactgcaaaccacctaaaattagcagagaaacggaagaacttctcaaactgattaaggaattagtcgccatcatcccgctcgttttcgcaaacccgtctgtggctaatgtaacttcattgcaacagattttacagcgattattagctctcgcaaataaattgagacttagaggctcggctaagacagatttattagcggcgttggaactggctatcgtggcgtcggaagccactcttttctccccgatcggtgttggaacgacactgcaacaactgctggaagtcttattgtctattattttgcaggaaccccttgatcctgctcttaaagacagtttgatcagtgcaatcagaaatgccgaaacggctatcagtattgcgttgggtggcacggcaggaacccccggtccacaagggcccgctgggcctgctggtccgggcggtgctccaggacctgtcggtggaccagggccggtgggtgcggcaggaccagcaggtccagttggacctgctggtcctgtcggacctgtcggggctgccggacctgttggagccgccggacctgttggagccgccggacctatcggcgccgctgggccagtaggcgccgccggggctgctggagccaccggggctacaggagctacaggcgcggcaggacctgccgggggggctaccggggccacgggcgccgttggagccacaggcgctacgggcgcagcgggggtcgctggggctacaggaactacgggcacggcgggcgctgtcggagctaccggggccacgggcacggcgggggccattggagctaccggggccacaggcacggcgggggccgtcggagctaccggggccacaggcacggcgggcgctgtcggagctaccggggccacgggtacagcaggggttactggagccaccggttcgggggcaatcattccatttgcttcgggtggaccagcaattttgacaaccattgtcggcgggctggttggaaccacaagtttgatcggctttggaagctcagcaacaggcattagccttgtgggtggaaccattgacctgacaggcacacttgcagggccactgattaactttgctttttctgtaccacgggatggcgtaattacatccatcgctggatattttagtacaacagctgcgctaactctcgttggatcaaccgcgacgattactgcccagttgtttagttcgactacacctgataacacctttacagcggtccctggggctaccgttacattagctccaccactgactggcatcattgccttgggtaccatttccaatggcatcactaccggattggctataccagtaaccgcgcagactcgtctgctccttgtcttctctgcaacagctacgggactctccctcgtaaacaccatcgtgggttatgcgagcgcaggcattaccatcacctga

SEQ ID NO: 26 - protein, 498 aa, Paenibacillus sp. NRRL B-50972
MKHRKPFRFSGASKKDEDCKPPKISRETEELLKLIKELVAIIPLVFANPSVANVTSLQQILQRLLALANKLRLRGSAKTDLLAALELAIVASEATLFSPIGVGTTLQQLLEVLLSIILQEPLDPALKDSLISAIRNAETAISIALGGTAGTPGPQGPAGPAGPGGAPGPVGGPGPVGAAGPAGPVGPAGPVGPVGAAGPVGAAGPVGAAGPIGAAGPVGAAGAAGATGATGATGAAGPAGGATGATGAVGATGATGAAGVAGATGTTGTAGAVGATGATGTAGAIGATGATGTAGAVGATGATGTAGAVGATGATGTAGVTGATGSGAIIPFASGGPAILTTIVGGLVGTTSLIGFGSSATGISLVGGTIDLTGTLAGPLINFAFSVPRDGVITSIAGYFSTTAALTLVGSTATITAQLFSSTTPDNTFTAVPGATVTLAPPLTGIIALGTISNGITTGLAIPVTAQTRLLLVFSATATGLSLVNTIVGYASAGITIT

SEQ ID NO: 27 - DNA, 3744 bp, Paenibacillus sp. NRRL B-50972
atggcggttatatcaactggacccatagaaaataattatgtcagtggtattcggcctactcatcgagttaccgtgaaaattgataatcgtgatactgtgaattcttctacggtattgattcagggtttttatctaaatggtacaagaacgttatatgtgcttgattttataactgtaaattcaaatgaagtgattacaaaagattattatgctgattttaattcatttgagtttgtttttaccactgaaagtgttacagaaaatgagattcaagtttcagtctggggtaaaaattcaatggggcagttagtgacagctcaccgtgttgtatcttccgaattgcttgtagcaaaaggcgcgggaccgacagggctaacgggagccactggcgctaccggagctactggcgtcacgggtgttaccggagtcactggcgctaccggaactacgggcgttatgggtgataccggagtcactggagttaccggagttactggcgttaccggggctatcggagtcactggcgctatcggagtcacgggggctaccggagccacaggagttacgggggccactggagttaccggggctattggagttactggcgctatcggagtcactggcgctaccggagctactggcgttactggggctactggcgctactggagtcacaggagttaccggggctactggcgttaccggagttaccggagttactggcatcaccggggctatcggagctactggcgttaccggagctactggcgtcacgggtattaccggagtcactggcgttaccggggctactggcgttactggagttactggcatcacaggcgttaccggagttactggtgttactggtgttactggagctactggcgttaccggggctactggcgctaccggagccactggcgttactggagttactggcgttactggcgctactggagctactggtgttaccggggctaccggggctaccggtgtcacgggtgataccggtgtcactggcgctaccggggctaccggagtttctggcgctactggggctactggtgtcacgggtgataccggagttaccggagctactggcgctacaggtgctaccggagttactggcggaacaggtgcaaccggagttactggagttactggcgttaccggggctatcggagtcactggcgctactggagctactggagctgctggaatcacgggtgttaccggagttactggcatcaccggtgctaccggggctacgggcgctaccggagttactggcatcacaggagtcactggcgctaccggagttactggcgtaacaggtgcaaccggagttactggagttaccggggctatcggagttactggtgtcaccggagctactggcgtcacgggtgttaccggagtcactggcgctaccggagctactggcgttacgggtgttaccggagttaccggagttactggcgttaccggagctactggcgttaccggagttactggagttactggagttattggagttactggagttactggagttactggagttactggagttaccggagttaccggagttactggagttaccggggctatcggagtcactggcgctatcggagtcacgggggctaccggggtcactggcgctaccggagctactggcgtaacaggggctactggagttaccggggctatcggagtcactggcgctactggagctgctggaatcacgggtgttaccggagtcactggtgttactggagttaccggagctactggcatcacgggtgataccggagtcactggcgctaccggagctactggcgttacgggtgttaccggagtcactggggctaccggagctactggcgtcacgggtgataccggagttactggagtcactggcgctaccggagttactggcgtaacaggtgcagccggagttactggcatcacgggggctaccggagttactggagttaccggggctattggagtcactggcgctatcggagtcacgggggctaccggagccacaggagttacgggtattaccggagctactggcgctactggagccacaggtgctaccggagttactggagttactggcgctaccggagctactggcgctactggcgtcacgggttctactggggtcactggcgctactggcgttaccggagctactggcgtcacgggttctactggggtcactggcgctactggcgttaccggagctactggcgtcacgggtattaccggagtcactggcgttaccggagttactggtgctactggagctactggcgttaccggggctaccggagtcactggggctaccggagctactggcgtcacgggtattaccggagtcactggggctaccggagctactggcgtcacgggtgttaccggagtcaccggagtcactggagttactggagttactggcgctaccggagctactggcgttaccggagctactggcgctactggcgtcacgggtgataccggagtcactggggctaccggagttaccggagtcactggcgctactggggctactggtgtcacgggtgttaccggagtcactggcgctaccggggctactggtgtcacgggtgttaccggggctaccggagctactggcgacacgggtgttaccggagtcactggagtcactggagttaccggagtttctggcgctaccggagttaccggagtttctggcgctaccggagttaccggagctactggcgttaccggggctggggctaccggagctactggcgctactggagtcacaggtgttaccggagtcactggcgctaccggagctactggcgctactggagtcacgggtgttaccggagtcactggcgctaccggggctactggtgtcacgggtgttaccggggctaccggagctactggcgacacgggtgttaccggagtcactggagtcactggagttaccggagtttctggcgctaccggagttaccggagctactggcgttaccggggctggggctaccggagctactggcgctactggagtcacaggtgttaccggagtcactggcgctaccggagctactggcgctactggagtcacgggtgttaccggagtcactggcgctaccggggctactggcgctactggagtcacgggtgttactggcgttacgggtgttaccggagtttctggcatcaccggtgctaccggggctattggacctactggtgccacaggtgttggtataacaggttcaacaggttcaaccggccccactggcccacctcctacgtttatagacgcatactttaacggtaatattcaacctcagacaattgcttcgggatcaaacattttaaatattactccaaaccaatctactgcacttacttataacgcagtaacaagtgttttcacaatacaaaatgcggggttgtataacattagtgttgtaataaatcttgcaactgccacactaccagaagcaacaattgggttatcactaaataattctacagcatatctcgctcctgctgtaaccacggcaacaagtggtcaattggttttagttcaaattgaggctcttgctgtcggagatacaattcaatttagaaatatatctgggtttcctattaccattgctaattcaccagtaatagctaacagctcaggtcatgtagctatttcgagattctcagctttttcataa

SEQ ID NO: 28 - protein, 1247 aa, Paenibacillus sp. NRRL B-50972
MAVISTGPIENNYVSGIRPTHRVTVKIDNRDTVNSSTVLIQGFYLNGTRTLYVLDFITVNSNEVITKDYYADFNSFEFVFTTESVTENEIQVSVWGKNSMGQLVTAHRVVSSELLVAKGAGPTGLTGATGATGATGVTGVTGVTGATGTTGVMGDTGVTGVTGVTGVTGAIGVTGAIGVTGATGATGVTGATGVTGAIGVTGAIGVTGATGATGVTGATGATGVTGVTGATGVTGVTGVTGITGAIGATGVTGATGVTGITGVTGVTGATGVTGVTGITGVTGVTGVTGVTGATGVTGATGATGATGVTGVTGVTGATGATGVTGATGATGVTGDTGVTGATGATGVSGATGATGVTGDTGVTGATGATGATGVTGGTGATGVTGVTGVTGAIGVTGATGATGAAGITGVTGVTGITGATGATGATGVTGITGVTGATGVTGVTGATGVTGVTGAIGVTGVTGATGVTGVTGVTGATGATGVTGVTGVTGVTGVTGATGVTGVTGVTGVIGVTGVTGVTGVTGVTGVTGVTGVTGAIGVTGAIGVTGATGVTGATGATGVTGATGVTGAIGVTGATGAAGITGVTGVTGVTGVTGATGITGDTGVTGATGATGVTGVTGVTGATGATGVTGDTGVTGVTGATGVTGVTGAAGVTGITGATGVTGVTGAIGVTGAIGVTGATGATGVTGITGATGATGATGATGVTGVTGATGATGATGVTGSTGVTGATGVTGATGVTGSTGVTGATGVTGATGVTGITGVTGVTGVTGATGATGVTGATGVTGATGATGVTGITGVTGATGATGVTGVTGVTGVTGVTGVTGATGATGVTGATGATGVTGDTGVTGATGVTGVTGATGATGVTGVTGVTGATGATGVTGVTGATGATGDTGVTGVTGVTGVTGVSGATGVTGVSGATGVTGATGVTGAGATGATGATGVTGVTGVTGATGATGATGVTGVTGVTGATGATGVTGVTGATGATGDTGVTGVTGVTGVTGVSGATGVTGATGVTGAGATGATGATGVTGVTGVTGATGATGATGVTGVTGVTGATGATGATGVTGVTGVTGVTGVSGITGATGAIGPTGATGVGITGSTGSTGPTGPPPTFIDAYFNGNIQPQTIASGSNILNITPNQSTALTYNAVTSVFTIQNAGLYNISVVINLATATLPEATIGLSLNNSTAYLAPAVTTATSGQLVLVQIEALAVGDTIQFRNISGFPITIANSPVIANSSGHVAISRFSAFS

SEQ ID NO: 29 - DNA, 849 bp, Paenibacillus sp. NRRL B-50972
ttgggaaatttattgttgcgtaaaagatatcgcttgacccaggtggcaaggaaaaaaaagaaggaaagagatcaaaagatgggagcgttccgttttatgcccatttatcgtacaggaacgagctgcattcgtaacaaaaagggaaataaacgtatttatagacagggtagaagaagagagagaatatgcgcttatagacatcatttgcacgctgagcgggtgccctcaggtttatcaaataaaaaaatctgttttatgaaattcaaaggtcaacgaagactgcgaggcggcgaacaggagcctcaaggcaattcaggaggagcagttcaaggggtgcatggattaagggggaccgatggtaatgctgggcatcaaggcatacaaggtccggctgggccacagggcattccgggtagtgccggaccccagggccaggcgggcgccataggcccccaaggtgaacagggtcttcagggggttccagggattcaaggcttgcaaggagaggctgggccacagggagagcagggaccaccgcttaatttggatgggatcacggttgtgcctgaggtacagcgatatttctattttgccgattcagatctgacgggtacggttgaaatccctatttcccagtttacgaatgatgatggacagttggcaagtcagcttccagaattgggtgcgaacagctacacggatttgtatattaatggggtactgcaggaaagcaggttgtaccagataagtagtaccacattgactgttgaattggaagaagctcttgtaattgcgggtacgccgtttattttcgaggtttttcaatttacattaagaatggcgaactga

SEQ ID NO: 30 - protein, 282 aa, Paenibacillus sp. NRRL B-50972
MGNLLLRKRYRLTQVARKKKKERDQKMGAFRFMPIYRTGTSCIRNKKGNKRIYRQGRRRERICAYRHHLHAERVPSGLSNKKICFMKFKGQRRLRGGEQEPQGNSGGAVQGVHGLRGTDGNAGHQGIQGPAGPQGIPGSAGPQGQAGAIGPQGEQGLQGVPGIQGLQGEAGPQGEQGPPLNLDGITVVPEVQRYFYFADSDLTGTVEIPISQFTNDDGQLASQLPELGANSYTDLYINGVLQESRLYQISSTTLTVELEEALVIAGTPFIFEVFQFTLRMAN

SEQ ID NO: 31 - protein, 60 aa, Artificial Sequence
  Consensus sequence
  misc_feature (2)..(2): Xaa can be any naturally occurring amino acid
  misc_feature (4)..(4): Xaa can be any naturally occurring amino acid
  misc_feature (10)..(10): Xaa can be any naturally occurring amino acid
  misc_feature (12)..(13): Xaa can be any naturally occurring amino acid
  misc_feature (15)..(15): Xaa can be any naturally occurring amino acid
  misc_feature (17)..(17): Xaa can be any naturally occurring amino acid
  misc_feature (21)..(22): Xaa can be any naturally occurring amino acid
  misc_feature (32)..(32): Xaa can be any naturally occurring amino acid
  misc_feature (37)..(37): Xaa can be any naturally occurring amino acid
  misc_feature (41)..(41): Xaa can be any naturally occurring amino acid
  misc_feature (44)..(44): Xaa can be any naturally occurring amino acid
  misc_feature (48)..(48): Xaa can be any naturally occurring amino acid
  misc_feature (54)..(58): Xaa can be any naturally occurring amino acid
  misc_feature (60)..(60): Xaa can be any naturally occurring amino acid
MXVXSTGPIXNXXVXGXRPTXXVTVKIDNRDXVNSSXVLIXGFXLNGXRTLYVXXXXXVX

SEQ ID NO: 32 - protein, 120 aa, Artificial Sequence
  Consensus Sequence
  misc_feature (2)..(2): Xaa can be any naturally occurring amino acid
  misc_feature (4)..(4): Xaa can be any naturally occurring amino acid
  misc_feature (10)..(10): Xaa can be any naturally occurring amino acid
  misc_feature (12)..(13): Xaa can be any naturally occurring amino acid
  misc_feature (15)..(15): Xaa can be any naturally occurring amino acid
  misc_feature (17)..(17): Xaa can be any naturally occurring amino acid
  misc_feature (21)..(22): Xaa can be any naturally occurring amino acid
  misc_feature (32)..(32): Xaa can be any naturally occurring amino acid
  misc_feature (37)..(37): Xaa can be any naturally occurring amino acid
  misc_feature (41)..(41): Xaa can be any naturally occurring amino acid
  misc_feature (44)..(44): Xaa can be any naturally occurring amino acid
  misc_feature (48)..(48): Xaa can be any naturally occurring amino acid
  misc_feature (54)..(58): Xaa can be any naturally occurring amino acid
  misc_feature (60)..(61): Xaa can be any naturally occurring amino acid
  misc_feature (63)..(63): Xaa can be any naturally occurring amino acid
  misc_feature (67)..(70): Xaa can be any naturally occurring amino acid
  misc_feature (72)..(75): Xaa can be any naturally occurring amino acid
  misc_feature (83)..(86): Xaa can be any naturally occurring amino acid
  misc_feature (90)..(90): Xaa can be any naturally occurring amino acid
  misc_feature (92)..(92): Xaa can be any naturally occurring amino acid
  misc_feature (98)..(100): Xaa can be any naturally occurring amino acid
  misc_feature (105)..(105): Xaa can be any naturally occurring amino acid
  misc_feature (109)..(109): Xaa can be any naturally occurring amino acid
  misc_feature (112)..(112): Xaa can be any naturally occurring amino acid
  misc_feature (117)..(120): Xaa can be any naturally occurring amino acid
MXVXSTGPIXNXXVXGXRPTXXVTVKIDNRDXVNSSXVLIXGFXLNGXRTLYVXXXXXVXXNXVITXXXXAXXXXFEFVFTTXXXXENEXQXSVWGKXXXGQLVXAHRXVSXELLVXXXX

SEQ ID NO: 33 - DNA, 300 bp, Paenibacillus sp. NRRL B-50972
atggtagtattatctactggacctattgcaaacgatcctgttctaggagtcagacccacccaactggtcacagtaaaaatagataaccgagattctgtaaattcttctatcgttttgatcgagggttttattttaaacggtagcagaacattatatgtacaacaattagtggtagtgggaccaaatgcggttataacgaggaatttctttgcaaatgtagacgcatttgaattcgtttttaccactagcggaccagcagagaatgaaactcaaatttctgtttggggtaaagatgcattg

SEQ ID NO: 34 - protein, 100 aa, Paenibacillus sp. NRRL B-50972
MVVLSTGPIANDPVLGVRPTQLVTVKIDNRDSVNSSIVLIEGFILNGSRTLYVQQLVVVGPNAVITRNFFANVDAFEFVFTTSGPAENETQISVWGKDAL

SEQ ID NO: 35 - DNA, 240 bp, Paenibacillus sp. NRRL B-50972
atggtagtattatctactggacctattgcaaacgatcctgttctaggagtcagacccacccaactggtcacagtaaaaatagataaccgagattctgtaaattcttctatcgttttgatcgagggttttattttaaacggtagcagaacattatatgtacaacaattagtggtagtgggaccaaatgcggttataacgaggaatttctttgcaaatgtagacgcatttgaattcgttttt

SEQ ID NO: 36 - protein, 80 aa, Paenibacillus sp. NRRL B-50972
MVVLSTGPIANDPVLGVRPTQLVTVKIDNRDSVNSSIVLIEGFILNGSRTLYVQQLVVVGPNAVITRNFFANVDAFEFVF

SEQ ID NO: 37 - DNA, 120 bp, Paenibacillus sp. NRRL B-50972
atggtagtattatctactggacctattgcaaacgatcctgttctaggagtcagacccacccaactggtcacagtaaaaatagataaccgagattctgtaaattcttctatcgttttgatc

SEQ ID NO: 38 - protein, 40 aa, Paenibacillus sp. NRRL B-50972
MVVLSTGPIANDPVLGVRPTQLVTVKIDNRDSVNSSIVLI

SEQ ID NO: 39 - DNA, 66 bp, Paenibacillus sp. NRRL B-50972
atgtttaccactagcggaccagcagagaatgaaactcaaatttctgtttggggtaaagatgcattg

SEQ ID NO: 40 - protein, 22 aa, Paenibacillus sp. NRRL B-50972
MFTTSGPAENETQISVWGKDAL

SEQ ID NO: 41 - DNA, 36 bp, Paenibacillus sp. NRRL B-50972
atgactcaaatttctgtttggggtaaagatgcattg

SEQ ID NO: 42 - protein, 12 aa, Paenibacillus sp. NRRL B-50972
MTQISVWGKDAL

SEQ ID NO: 43 - DNA, 21 bp, Paenibacillus sp. NRRL B-50972
atgactcaaatttctgtttgg

SEQ ID NO: 44 - protein, 7 aa, Paenibacillus sp. NRRL B-50972
MTQISVW

SEQ ID NO: 45 - DNA, 27 bp, Paenibacillus sp. NRRL B-50972
atgcaaatttctgtttggggtaaagat

SEQ ID NO: 46 - protein, 9 aa, Paenibacillus sp. NRRL B-50972
MQISVWGKD

SEQ ID NO: 47 - DNA, 21 bp, Paenibacillus sp. NRRL B-50972
atgtggggtaaagatgcattg

SEQ ID NO: 48 - protein, 7 aa, Paenibacillus sp. NRRL B-50972
MWGKDAL

SEQ ID NO: 49 - protein, 9 aa, Artificial Sequence
  Truncated N-terminal targeting sequence derived from Paenibacillus
MQISVWGKB